(19) United States
(12) Patent Application Publication
(10) Pub. No.: US 2009/0275064 A1
(43) Pub. Date: Nov. 5, 2009

(54) Methods to identify therapeutic agents

(75) Inventor: Paul Wentworth, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 12/459,618

(22) Filed: Jul. 1, 2009

Related U.S. Application Data
(62) Division of application No. 11/464,415, filed on Aug. 14, 2006.
(60) Provisional application No. 60/708,316, filed on Aug. 15, 2005.

(51) Int. Cl. C12Q 1/60; C07C 311/16

(52) U.S. Cl. 435/11; 564/86

(57) ABSTRACT

As illustrated herein, cholesterol is oxidized when it is present in atherosclerotic plaques. This reaction generates cholesterol oxidation or ozonation products that can act as chemotactic attractants of macrophages, can promote differentiation of monocytes into macrophages and can increase expression of E-selectin and Class A scavenger receptor (SR-A). The present application is directed to methods of using such cholesterol ozonation products to identify agents that can be used to treat atherosclerosis and other inflammatory artery diseases.

### RELATED APPLICATIONS

This application is a divisional of pending U.S. patent application Ser. No. 11/464,415, filed Aug. 14, 2006, which claims priority to U.S. Provisional Application Ser. No. 60/708,316, filed Aug. 15, 2005. The contents of these priority applications are incorporated herein in their entireties and for all purposes. This application is also related to U.S. Provisional Application Ser. No. 60/500,845 filed Sep. 5, 2003, to U.S. Provisional Application Ser. No. 60/517,940 filed Nov. 6, 2003, to U.S. application Ser. No. 10/934,319 filed Sep. 3, 2004, and to U.S. application Ser. No. 10/934,795 filed Sep. 3, 2004, the disclosures of which are incorporated herein in their entireties.

### STATEMENT OF GOVERNMENT RIGHTS

The invention described herein was made with United States Government support under Grant Number POCA 27489 awarded by the National Institutes of Health. The United States Government has certain rights in this invention.

### FIELD OF THE INVENTION

The invention relates to methods for identifying agents useful for treating and preventing atherosclerosis and/or cardiovascular disease by counteracting the effects of cholesterol ozonation products that are produced in atherosclerotic lesions. According to the invention, cholesterol ozonation products are cytotoxins that alter the differentiation, expression patterns, and/or chemotaxis of key cells involved in the development of atherosclerosis.

### BACKGROUND OF THE INVENTION

Cardiovascular disease remains, in most countries, one of the main diseases and the main cause of mortality. Approximately one third of men develop a major cardiovascular disease before the age of 60. While women initially exhibit a lower risk (ratio of 1 to 10), cardiovascular disease becomes more prevalent with age. For example, after the age of 65, women become just as vulnerable to cardiovascular diseases as men. Vascular diseases, such as coronary disease, strokes, restenosis and peripheral vascular disease, remain some of the mains cause of mortality and handicap across the world.

While physicians encourage changes in diet and lifestyle to reduce the development of cardiovascular diseases, a genetic predisposition leading to dyslipidaemias is a significant factor in the incidence of stroke and death from vascular disease. Accordingly, new insight into the formation and treatment of problematic atherosclerotic lesions is needed

### SUMMARY OF THE INVENTION

The inventors have previously shown that reactive oxygen species such as ozone are generated by antibodies. Wentworth et al., Science 298, 2195 (2002); Babior et al., Proc. Natl. Acad. Sci. U.S.A. 100, 3920 (2003); P. Wentworth Jr. et al., Proc. Natl. Acad. Sci. U.S.A. 100, 1490 (2003). This application provides evidence showing that reactive oxygen species such as ozone and cholesterol ozonation products are generated by atherosclerotic plaque materials.

According to the invention, ozonation products of cholesterol are present in atherosclerotic plaques and can exacerbate or accelerate the development of problematic plaque buildup. For example, ozonation products of cholesterol can promote lipid uptake by macrophages and accelerate the rate at which foam cells are formed. Ozonation products of cholesterol can also adversely affect the secondary structure of and apoprotein B₁₀₀ as well as the low density lipoproteins (LDLs) in which apoprotein B₁₀₀ is found. In addition, ozonation products of cholesterol can also modify the differentiation, expression patterns, or chemotaxis of cells involved in the development of atherosclerosis.

Thus, one aspect of the invention involves identifying agents that can counteract or inhibit the activity of cholesterol ozonation products. For example, in one embodiment, the invention is directed to a method for identifying an agent that can inhibit foam cell development in atherosclerotic tissues. This method involves contacting a macrophage with a test agent and observing whether expression of Class A scavenger receptor (SR-A) increases in the macrophage after exposing the macrophage to a cholesterol ozonolysis product. In some embodiments, the cell is exposed to cholesterol ozonolysis products 4a or 5a in the presence of LDL.

Another aspect of the invention is a method for identifying an agent that can inhibit recruitment of macrophages to atherosclerotic tissues. This method involves contacting a macrophage with a test agent and observing whether the macrophage migrates toward a source of a cholesterol ozonolysis product. In some embodiments, cholesterol ozonolysis products 4a or 5a are used as the cholesterol ozonolysis product.

Another aspect of the invention is a method for identifying an agent that can inhibit atherosclerosis. This method involves contacting an endothelial cell with a test agent and observing whether expression of E-selectin increases in the endothelial cell exposing the endothelial to a cholesterol ozonolysis product. In some embodiments, the cell is exposed to cholesterol ozonolysis products 4a or 5a in the presence of LDL.

Another aspect of the invention is a method for identifying an agent that can inhibit monocyte differentiation into macrophages. This method involves contacting a monocyte with a test agent and observing whether the monocyte differentiates into a macrophage, wherein the monocyte is cultured with cholesterol ozonolysis product. In some embodiments, the monocyte is cultures with cholesterol ozonolysis product 4a or 5a.

As provided by the invention, cholesterol ozonation products are markers for atherosclerotic lesions. Antibodies that do not generate ozone, as well as other binding agents that bind to ozonation products of cholesterol, can be used to inactivate or inhibit the toxicity of the ozonation products of cholesterol and thereby treat and prevent atherosclerosis. The invention therefore provides antibodies and binding entities directed against cholesterol ozonation products.

The invention is also directed to a method of treating or preventing atherosclerosis in a mammal by administering to the mammal an antibody or binding entity that has a therapeutic agent linked thereto, wherein the antibody or binding entity can bind to a molecule or antigen that is present in atherosclerotic plaque, for example, a cholesterol ozonation product. Such therapeutic agents can, for example, help slow the growth or reduce the size of the atherosclerotic lesion.

This application is also directed to the cytotoxic products of cholesterol ozonation, and methods of using such cytotoxic cholesterol ozonation products for treatment of autoimmune diseases, cancer, tumors, bacterial infections, viral infections, fungal infections, ulcers and/or other diseases where localized administration of a cytotoxin is beneficial.

One aspect of the invention is an isolated ozonation product of cholesterol that can be cytotoxic to a prokaryotic or eukaryotic cell. Such an ozonation product can cause macrophage lipid uptake or foam cell formation. The ozonation products of the invention can also change the secondary structure of a protein in a low density lipoprotein. For example, the ozonation products of the invention can change the secondary structure of apoprotein B₁₀₀.

The ozonation products of the invention include any compound having any one of formulae 4a-15a, 7c or a combination thereof.

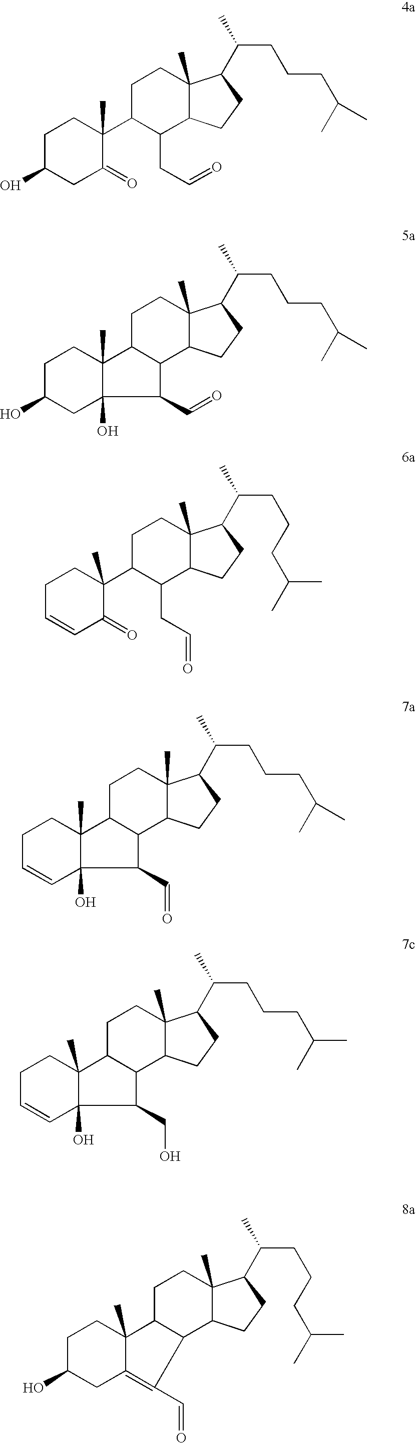

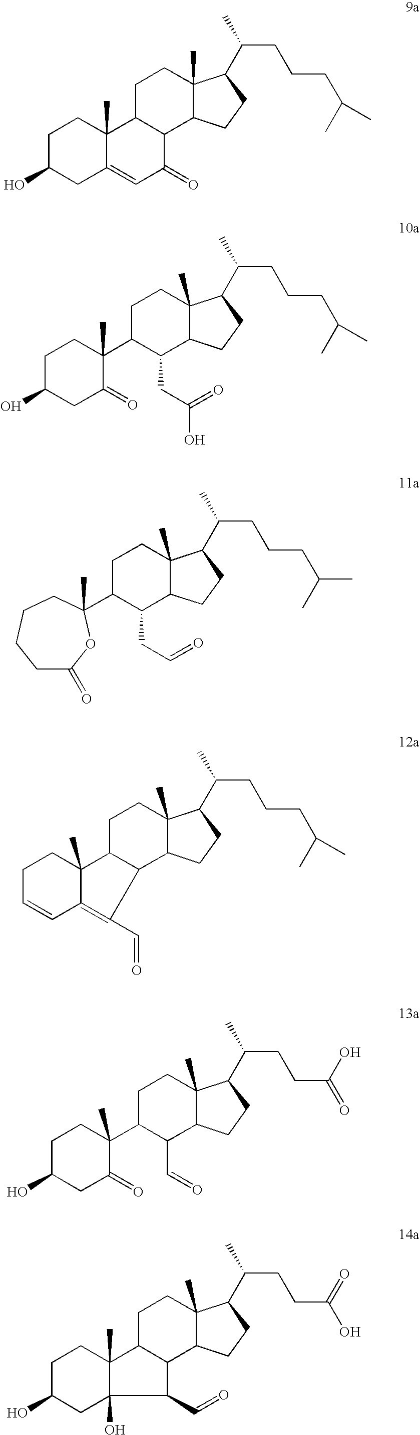

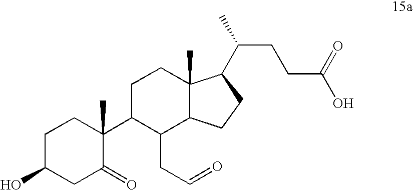

Another aspect of the invention is a marker for treating or preventing atherosclerotic lesions comprising an ozonation product of cholesterol having formula 4a or formula 5a.

Another aspect of the invention is a composition that includes a carrier and an isolated ozonation product of cholesterol that can be cytotoxic to a prokaryotic or eukaryotic cell. The ozonation product of cholesterol can be any of the ozonation products of cholesterol described herein.

Another aspect of the invention is an isolated binding entity that can bind to an ozonation product of cholesterol. The ozonation product of cholesterol to which the binding entity can bind can, for example, be any compound having any one of formulae 4a-15a, 7c or a combination thereof. In some embodiments, the ozonation product is 4a or 5a. The binding entity can, for example, be an antibody. The binding entity can be raised against a hapten, for example, a hapten having formula 13a, 14a or 15a. Examples of antibody binding entities include antibodies derived from hybridoma KA1-11C5 or KA1-7A6 having ATCC Accession No. PTA-5427 or PTA-5428. Other examples of antibody binding entities include antibodies derived from hybridoma KA2-8F6 or KA2-1E9, having ATCC Accession No. PTA-5429 and PTA-5430.

In some embodiments, the binding entities of the invention are linked to a therapeutic agent. The therapeutic agent employed can, for example, reduce an atherosclerotic lesion or prevent further occlusion of the artery. Examples of therapeutic agents that can be used with the binding agents of the invention include an anti-oxidant, anti-inflammatory agent, drug, small molecule, peptide, polypeptide or nucleic acid.

Another aspect of the invention is an isolated binding entity linked to an ozonation product of cholesterol, wherein the ozonation product of cholesterol is cytotoxic to a prokaryotic or eukaryotic cell.

Another aspect of the invention is a method for treating atherosclerosis in a patient comprising administering to the patient a binding agent that can bind to an ozonation product of cholesterol. The ozonation product of cholesterol to which the binding agent binds can be a compound having any one of formulae 4a-15a or 7c. Preferably, the binding agent does not generate a reactive oxygen species. In some embodiments, the binding entity is linked a therapeutic agent. Such therapeutic agents can help slow the growth or reduce the size of an atherosclerotic lesion. Examples of therapeutic agents that can be used include an anti-oxidant, anti-inflammatory agent, drug, small molecule, peptide, polypeptide or nucleic acid.

Another aspect of the invention is a method for killing a target cell in a patient by administering to the patient a binding agent that can bind to the target cell, wherein the binding agent is linked to an ozonation product of cholesterol. Such a binding entity can be an antibody. In this embodiment, the binding entity or antibody can generate a reactive oxygen species. The antibody can also be linked to a compound that can generate singlet oxygen. Examples of compounds that can generate singlet oxygen include endoperoxides such as an anthracene-9,10-dipropionic acid endoperoxide. Other examples of compounds that can generate singlet oxygen include a compound such as a pterin, flavin, hematoporphyrin, tetrakis(4-sulfonatophenyl)porphyrin, bipyridyl ruthenium(II) complex, rose Bengal dye, quinone, rhodamine dye, phthalocyanine, hypocrellin, rubrocyanin, pinacyanol or allocyanine.

Another aspect of the invention is a method for removing cytotoxic cholesterol ozonation products from a mammal by separating the cytotoxic cholesterol ozonation products from bodily fluids of the mammal using a binding entity or an antibody that can bind to an ozonation product of cholesterol. The ozonation product can be removed from circulating blood of the mammal. In another embodiment, the ozonation product is removed ex vivo from blood of the mammal. In a further embodiment, the binding entity or the antibody is administered in a localized manner to the localized tissues.

Another aspect of the invention is a method of treating or preventing cancer in a mammal by administering to the mammal an antibody linked to a cytotoxic ozonation product of cholesterol, wherein the antibody can bind to a cancer cell.

Another aspect of the invention is a method of treating or preventing an inappropriate immune response in a mammal by administering to the mammal an antibody linked to a cytotoxic ozonation product of cholesterol, wherein the antibody can bind to an immune cell involved in the inappropriate immune response.

Another aspect of the invention is a method for identifying an agent that modulates the production of a reactive oxygen species from an antibody by: (a) combining an antibody and a candidate agent; (b) determining the amount of reactive oxygen species formed; and (c) comparing the amount of reactive oxygen species formed with a standard value obtained by determining the amount of reactive oxygen species formed from the antibody without the candidate agent. In some embodiments, the reactive oxygen species is ozone.

### DESCRIPTION OF THE DRAWINGS

FIG. 1A-D shows that indigo carmine 1 can be oxidized to form isatin sulfonic acid 2 by 4-β-phorbol 12-myristate 13-acetate (PMA)-treated human atherosclerotic lesions.

FIG. 1A illustrates the chemical changes occurring during conversion of indigo carmine 1 into isatin sulfonic acid 2 by ozone.

FIG. 1B illustrates bleaching of indigo carmine 1 by a PMA-activated atherosclerotic lesion. Each glass vial contained equal amounts of a dispersion of atherosclerotic plaque (about 50 mg wet weight) in a solution of indigo carmine 1 (200 μM) and bovine catalase (50 μg) in phosphate buffered saline (PBS, 10 mM sodium phosphate, 150 mM NaCl) pH 7.4. The photograph was taken 30 min after the addition of a solution of PMA (10 μL, 40 μg/mL) in DMSO to the vial on the right. DMSO of the same volume without PMA was added to the vial on the left. The total volume of reaction mixture was 1 mL.

FIG. 1C shows that a new HPLC peak arises in the supernatant of the +PMA vial shown in FIG. 1B, as analyzed by reversed-phase HPLC. The new peak corresponds to isatin sulfonic acid 2, having a retention time (R_{T}) of about 9.71 min.

FIG. 1D shows a negative ion electrospray mass spectrograph of a supernatant from centrifuged PMA-activated human atherosclerotic plaque material reacted with indigo carmine 1 as described above for FIG. 1B. When PMA activation of suspended plaque material was performed in H₂¹⁸O using the indicator indigo carmine 1, approximately 40% of the lactam carbonyl oxygen of indigo carmine 1 incorporated ¹⁸O, as shown by the appearance and relative intensity of the [M−H]⁻ 230 mass fragment peak in the mass spectrum of the isolated cleaved product isatin sulfonic acid 2. Isatin sulfonic acid 2 formed from indigo carmine 1 in the presence of normal water (H₂¹⁶O) has a mass fragment peak [M−H]⁻ of 228.

FIG. 2A illustrates the chemical steps involved in the ozonolysis of cholesterol 3 to give 5,6-secosterol 4a that can be converted by aldolization into 5a. Derivatization with 2,4-dinitrophenylhydrazine (2 mM in 0.08% HCl) furnished the hydrazone derivatives 4b and 5b respectively. The amount of 5b formed from 4a during the derivatization process was about 20%. The conformational assignments of 5a and 5b were assigned as described by K. Wang, E. Bermúdez, W. A. Pryor, Steroids 58, 225 (1993).

FIG. 2B shows the structures of oxysterols 6a-9a and 2,4-dinitrophenylhydrazine hydrochloride derivatives 6b-7b investigated as standards for the peak eluting at about 18 min [M−H]⁻ 579 in FIG. 3. The conformational assignments of 7a-7b were based on a ¹H-¹H ROESY experiment using authentic synthetic 7b material.

FIG. 3A-E illustrate an analysis of plaque material and chemically synthesized authentic samples of hydrazones 4b, 5b and 6b using liquid chromatography mass spectroscopy (LCMS). Conditions: Adsorbosphere-HS RP-C18 column, 75% acetonitrile, 20% water, 5% methanol, 0.5 mL/min flow rate, 360 nm detection, in-line negative ion electrospray mass spectrometry (MS) (Hitachi M8000 machine) of a plaque extract after derivatization with 2,4-dinitrophenylhydrazine hydrochloride (DNPH HCl).

FIG. 3A illustrates an LCMS analysis of a plaque material without PMA activation but after derivatization with 2,4-dinitrophenylhydrazine as described herein. Compounds 4b (RT˜14.1 min), 5b (RT˜20.5 min) and 6b (RT˜18 min) were detected in an atherosclerotic lesion before activation with PMA (40 g/mL).

FIG. 3B illustrates an LCMS analysis of plaque material after activation with PMA (40 μg/mL), extraction and derivatization with 2,4-dinitrophenylhydrazine as described above. Larger amounts of compound 4b (RT˜14.1 min), but smaller amounts of compound 6b (RT˜18 min) were detected in an atherosclerotic lesion after activation with PMA (40 μg/mL).

FIG. 3C illustrates an HPLC analysis of authentic 4b; the inset shows the mass spectroscopy analysis.

FIG. 3D illustrates an HPLC analysis of authentic 6b; the inset shows the mass spectroscopy analysis.

FIG. 3E illustrates an HPLC analysis of authentic 5b; the inset shows the mass spectroscopy analysis.

FIG. 4A-D illustrate HPLC-MS analyses of extracted and derivatized atherosclerotic material where a 100 μl injection volume was used to allow detection of trace hydrazones. FIG. 4A shows a LC trace of time versus intensity using the conditions detailed vide supra. R_{T} 26.7 is 7b (by comparison to authentic material). The peak at R_{T}˜24.7 is an unknown hydrazone with [M−H]⁻ 461. FIG. 4B provides a single ion monitoring of [M−H]⁻ 597. FIG. 4C provides a single ion monitoring of [M−H]⁻ 579. FIG. 4D shows a single ion monitoring of [M−H]⁻ 461.

FIG. 5A-C illustrates the concentrations of cholesterol ozonation products in atherosclerotic extracts for patients A-N.

FIG. 5A is a bar chart showing the measured concentration of hydrazone 4b after extraction and derivatization of 4a from atherosclerotic lesions of patients, pre- and post-activation with PMA. The bar chart shows the numerical values of the amounts detected before and after activation as determined by a Student t-test (two-tail) (p<0.05, n=14) analysis using GraphPad Prism V3 for Macintosh.

FIG. 5B is a bar chart showing the measured concentration of 5b after extraction and derivatization of 5a from atherosclerotic lesions of patients, pre- and post-activation with PMA (n=14).

FIG. 5C is a bar chart showing measured concentrations of 5b after extraction and derivatization of 5a from plasma samples taken from patients. Cohort A (n=8) patients were to undergo a carotid endarterectomy procedure within 24 h (plasma analysis was performed 3 days after sample collection). Cohort B (n=15) patients were randomly selected from patients attending a general medical clinic (plasma analysis was performed 7 days after sample collection). Note that in a preliminary investigation plasma levels of 5a, fall by about 5% per day. Under the conditions of this assay, the detection limit of 4b and 5b was 1-10 nM. Therefore, in cases where no 4b or 5b was apparent, the level of 4b or 5b was less than 10 nM.

FIG. 6A illustrates the cytotoxicity of 3, 4a and 5a against B-cell (WI-L2) cell line. Each data point is the mean of at least duplicate measurements. The IC₅₀±standard errors for 4a (▪) and 5a (▴) were calculated using non-linear regression analysis (Hill plot analysis), with GraphPad Prism v 3.0 for the Macintosh computer. No cytotoxicity with 3 (▾) was observed in this concentration range.

FIG. 6B illustrates the cytotoxicity of 3, 4a and 5a against T-cell (Jurkat) cell line. Each data point is the mean of at least duplicate measurements. The IC₅₀s±standard errors for 4a (▪) and 5a (▴) were calculated using non-linear regression analysis (Hill plot analysis), with GraphPad Prism v 3.0 for the Macintosh computer. No cytotoxicity with 3 (▾) was observed in this concentration range.

FIG. 7A-B shows that of cholesterol ozonolysis products 4a and 5a increase lipid-loading by macrophages to produce foam cells.

FIG. 7A shows that LDL incubated with J774.1 macrophages has little effect upon lipid-loading of those macrophages. Macrophages were first grown for 24 h in RPMI-1640 containing 10% fetal bovine serum and then incubated for 72 h in the same media containing LDL (100 μg/mL). Cells were fixed with 4% formaldehyde and stained with hematoxylin and oil red O such that lipid granules stained a darker red color. Magnification×100.

FIG. 7B shows that LDL incubated with ozonolysis product 4a induces lipid-loading of macrophages to produce foam cells. J774.1 macrophages were grown for 24 h in RPMI-1640 containing 10% fetal bovine serum. Cells were then incubated for 72 h in the same media containing LDL (100 μg/mL) and ozonolysis product 4a (20 μM). Cells were fixed with 4% formaldehyde and stained with hematoxylin and oil red 0 such that lipid granules stained a darker red color. Magnification×100. Note that the effect of ozonolysis product 4a upon macrophages was indistinguishable from the effect of ozonolysis product 5a.

FIG. 8A-C shows that the secondary structure of LDL is altered by exposure to ozonolysis product 4a or 5a, as detected by circular dichroism. Results reported are from at least duplicate experiments for each sample.

FIG. 8A shows that the protein content of normal LDL has a large proportion of a helical structure (˜40±2%) and smaller amounts of β structure (˜13±3%), P turn (˜20±3%) and random coil (27±2%). FIG. 8A shows time-dependent circular dichroism spectra of LDL (100 μg/ml) at 37° C. in PBS (pH 7.4).

FIG. 8B shows that incubation of LDL with ozonolysis product 4a in PBS (pH 7.4) at 37° C. leads to a loss of secondary structure of apoB-100. FIG. 8A shows time-dependent circular dichroism spectra of LDL (100 μg/ml) and 4a (10 μM) at 37° C. in PBS (pH 7.4).

FIG. 8C shows that incubation of LDL with ozonolysis product 5a in PBS (pH 7.4) at 37° C. leads to a loss of secondary structure of apoB-100. FIG. 8A shows time-dependent circular dichroism spectra of LDL (100 μg/ml) and 5a (10 μM) at 37° C. in PBS (pH 7.4).

FIG. 9 illustrates the structures for dansyl hydrazine cholesterol ozonation products 4a and 5a (4d and 5c, respectively) and the HPLC elution patterns of these hydrazine derivatives. As shown, cholesterol ozonation products 4a and 5a give rise to dansyl hydrazone conjugates having different HPLC retention times.

FIG. 10 illustrates that cholesterol ozonation products can be detected in human carotid artery specimens by gas chromatography-mass spectroscopy (GCMS) analysis. The chromatogram shown is typical of atherosclerotic plaque extracts. The peak eluting at 22.49 minutes is the peak corresponding to both cholesterol ozonation products 4a and 5a. The insert mass spectrometry chromatograph illustrates that the species eluting at 22.49 minutes has m/z 354.

FIG. 11 provides a quantitative analysis of two atherosclerotic plaques (P1 and P2) by ID-GCMS. The amounts of cholesterol ozonation products 4a and 5a detected were about 80-100 pmol/mg tissue and were similar to those detected by LC-MS analysis. Each bar represents a duplicate extract and is reported as the mean±SEM.

FIG. 12A-D illustrate the uptake and localization of the dansyl derivative of cholesterol ozonation product 5e. Fluorescence microscopy of cultured macrophage cells (J774A.1) treated with 5e (50 μM) in PBS for 5 min (100×, FIG. 12A) and 1 h (200×, FIG. 12B). FIG. 12C shows cultured macrophages treated with cholesterol ozonation product 5e (50 μM) in media with FCS (10%) for 5 min (60×). FIG. 12D shows cultured macrophages (RAW 264.7) treated with cholesterol ozonation product 5e (50 μM) in media with FCS (10%) for 60 min (100×). Control samples and cells treated with compound 9d or a dansyl amide compound used to synthesize 5e exhibited minimal fluorescence (data not shown). After treatment, cells were fixed in 95% cold methanol and mounted in glycerol. These data were collected using a 60× oil immersion objective lens (NA 1.4) and a filter set combination (excitation) DAPI 360/40 and (emission) 457/50.

FIG. 13 illustrates the effects of cholesterol ozonation product 4a and 5a on macrophage scavenger receptor expression. As illustrated, cholesterol ozonation product 4a and 5a complexed with LDL increased SR-A expression but had little or no effect on CD36 expression. Macrophage cells (J774A.1) were treated with vehicle, LDL, Cu-OxLDL, 25 μM cholesterol ozonation product 4a (referred to as atheronal A) with LDL (100 μg/ml protein) or 25 μM cholesterol ozonation product 5a/(referred to as atheronal B) with LDL (100 μg/ml protein) for 6 h. Untreated control samples exhibited minimal fluorescence (CD36/FITC 12.7±0.5; SR-A/PE 31.7±4.1). Data represent mean fluorescence intensity±SEM of two individual experiments *P<0.05,**P<0.01 versus control.

FIG. 14A-B illustrate macrophage chemotaxis towards a source of cholesterol ozonolysis products 4a (referred to as atheronal A) and 5a (referred to as atheronal B). J774A.1 macrophages were treated with either cholesterol (25 μm), C5a (10 nM), ozonolysis product 4a (25 μM), or ozonolysis product 5a (25 μM) in chemotaxis chambers. FIG. 14A shows the migrated cells per microscopic field in the chamber with cholesterol, C5a, ozonolysis product 4a or ozonolysis product 5a. Cells were counted using a light microscope and expressed as cells per high-power field. A total of 15 high-power fields were counted for each sample. Cholesterol stimulated migration similar to vehicle control (41±8 cells/field). FIG. 14B graphically illustrates that the fluorescence of cells in migration chambers containing ozonolysis product 5a increases with ozonolysis product concentration. Calcein-AM labeled cells were incubated in the presence of different concentrations of ozonolysis product 5a in the lower chambers. Fluorescence of migrated cells was measured by fluorescence plate reader and expressed as number of migrated cells/well. Shown are mean±SEM values of 3 experiments. **P<0.001 versus control.

FIG. 15A-B graphically illustrates expression of adhesion molecules in vascular endothelial cells in the presence of ozonolysis product 4a (atheronal A) or 5a (atheronal B) complexed with LDL. FIG. 15A shows the effects of these cholesterol ozonolysis products at a single concentration. FIG. 15B shows the effect of increasing concentrations of LDL ozonolysis product 4a upon E-selectin expression. For FIG. 15A, HAAE-1 endothelial cells were incubated with LDL, Cu-oxLDL, ozonolysis product 4a/LDL or ozonolysis product 5a/LDL (100 μg/mL protein) for 4 h. Surface expression of VCAM-1, E-selectin and ICAM-1 were measured by ELISA. Data shown are mean±SEM from two separate experiments of the percent expression relative to the vehicle (100%). *P<0.05, **P<0.005 versus vehicle. For FIG. 15B, cultured endothelial cells (HAAE-1) were incubated with a range of ozonolysis product 4a (atheronal A) concentrations (0-50 μM) complexed with LDL (100 μg/mL protein), and the surface expression of E-selectin was measured as described for FIG. 15A. Data are reported as the mean±SEM of triplicate experiments.

FIG. 16A-H illustrates cholesterol ozonolysis product-induced monocyte differentiation into macrophages. THP-1 suspension cells were treated with either 12.5 or 25 μM of the following reagents for a period of 7 days: cholesterol (FIG. 16A-B), 7-ketocholesterol (positive control, FIG. 16C-D), ozonolysis product 4a (atheronal A, FIG. 16E-F), ozonolysis product 5a (atheronal B, FIG. G-H), THP-1 cells began to adhere after 4 days of treatment with 7-KC or ozonolysis product 5a (atheronal-B). Maximal adherence was observed after 7 days. In contrast, cholesterol and ozonolysis product 4a treatment induced no cell adherence over the same time frame. Representative phase-contrast microscopy images are shown.

### DETAILED DESCRIPTION OF THE INVENTION

According to the invention, ozonation products of cholesterol are present in atherosclerotic plaques. Those ozonation products of cholesterol can exacerbate or accelerate the development of atherosclerosis, for example, by recruiting macrophages to atherosclerotic tissues, increasing monocyte differentiation into macrophages, and altering the expression of a cell adhesion molecule (E-selectin) or of a Class A scavenger receptor (SR-A). The SR-A receptor is a key cell surface receptor responsible for macrophage internalization of modified LDL and oxysterols. In addition, ozonation products of cholesterol can modify the structure of apoprotein B₁₀₀ as well as the structure of low density lipoproteins (LDLs) in which apoprotein B₁₀₀ is found, by accelerating lipid uptake by macrophages, and increasing the number of foam cells formed. Hence, ozonation products of cholesterol can accelerate the formation of advanced atherosclerotic lesions that are more likely to lead to problematic symptoms of vascular disease, for example, heart attack, congestive heart failure, stroke and the like.

The invention also provides ozonation products of cholesterol that are useful as markers of atherosclerosis. Also provided are compositions, kits and binding agents that can counteract the effects of ozonation products of cholesterol. These compositions, kits and binding agents are useful for treating and preventing atherosclerosis, cardiovascular disease and other vascular diseases.

In another embodiment, the invention provides ozonation products of cholesterol as cytotoxins and methods for using these cytotoxic ozonation products to treat autoimmune diseases, cancer, tumors, bacterial infections, viral infections, fungal infections, ulcers and/or other diseases where localized administration of a cytotoxin is beneficial.

Another aspect of the invention is a method for identifying an agent that can inhibit cholesterol ozonolysis product activity upon a mammalian cell. This method involves contacting a cell with a test agent and observing whether the cell's differentiation, expression patterns, or chemotaxis changes, wherein the cell is cultured in the presence of a cholesterol ozonation product. In some embodiments, the cell is cultured with cholesterol ozonolysis product 4a or 5a.

### Cholesterol Ozonation

According to the invention, cholesterol is oxidized within atherosclerotic plaque material by reactive oxygen species such as ozone. A number of cholesterol ozonation products are generated by this process and can be detected in tissue or fluid samples taken from patients suffering from atherosclerosis.

Cholesterol has the following structure (3).

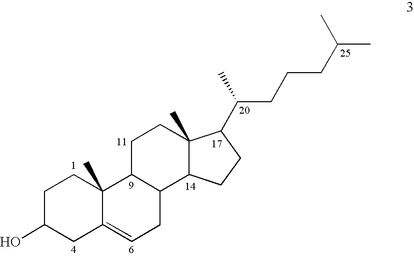

When cholesterol is laid down in an artery an atherosclerotic plaque can form. As illustrated herein atherosclerotic plaque can release reactive oxygen species such as ozone; such atherosclerotic plaque material also generates cholesterol ozonation products. While not wishing to be limited to a specific mechanism, it appears that macrophages, neutrophils, antibodies and other immune cells become enmeshed within the atherosclerotic lesion and release reactive oxygen species such as ozone. The reactive oxygen species produced react with the cholesterol in the lesion and oxidize the cholesterol into a number of products that can be detected in biological samples taken from patients.

For example, when cholesterol 3 is oxidized, the seco-ketoaldehyde 4a and its aldol adduct 5a are the main products formed.

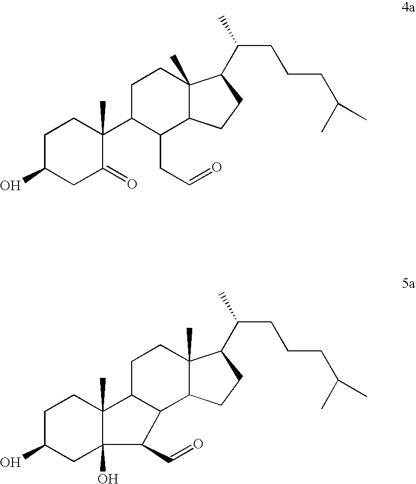

In addition, cholesterol ozonation products having structures like those of compounds 6a-15a, and 7c can also be observed.

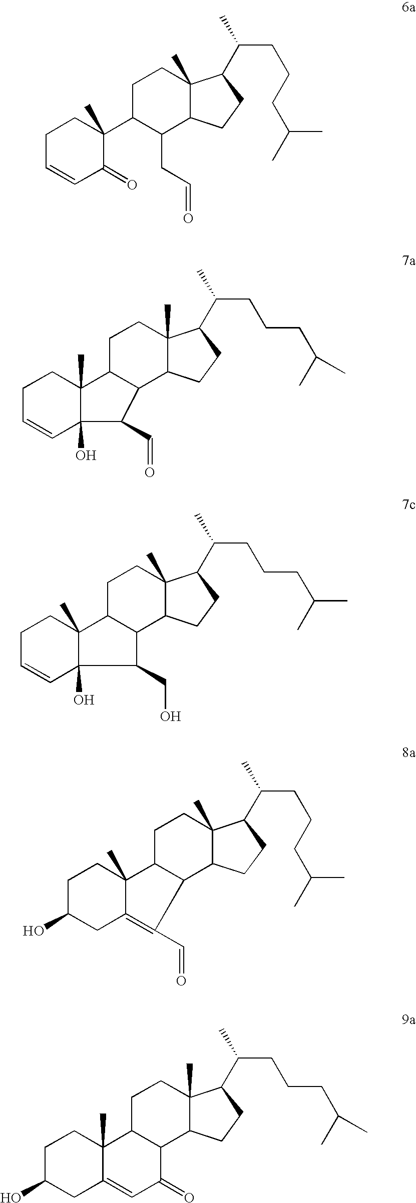

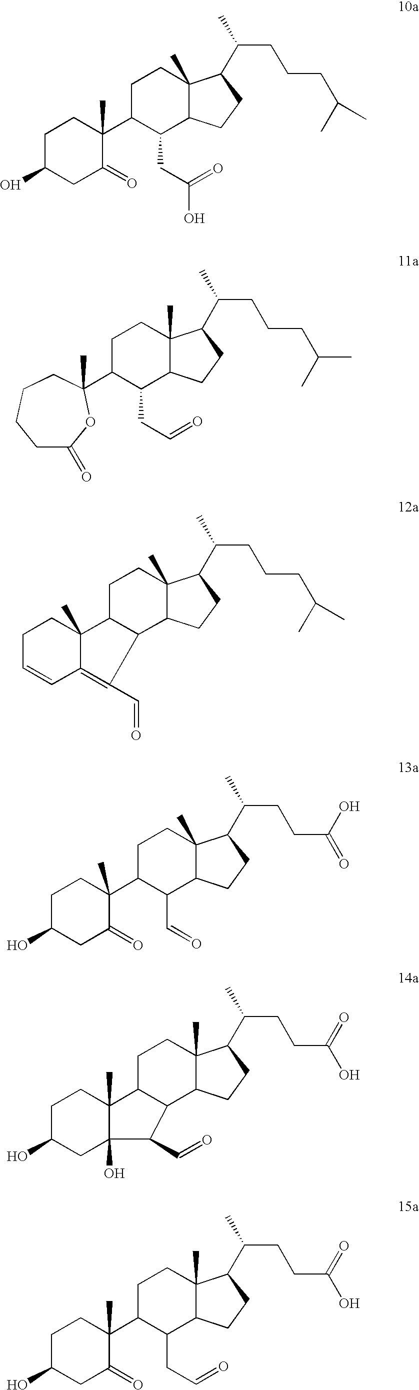

According to the invention, the seco-ketoaldehyde 4a, its aldol adduct 5a and related compounds such as 6a-15a or 7c are present in atherosclerotic plaque material taken from patients suffering from atherosclerosis. Moreover, the amount of the seco-ketoaldehyde 4a, aldol adduct 5a and the related compounds 6a-15a or 7c detected in the bloodstream of a patient is correlated with the extent and severity of atherosclerotic plaque formation in that patient.

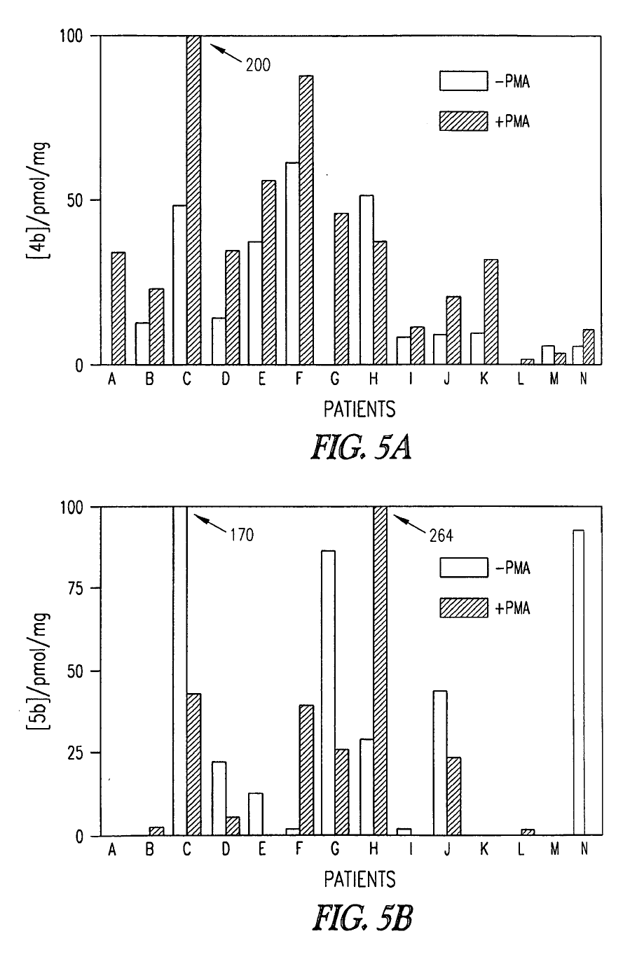

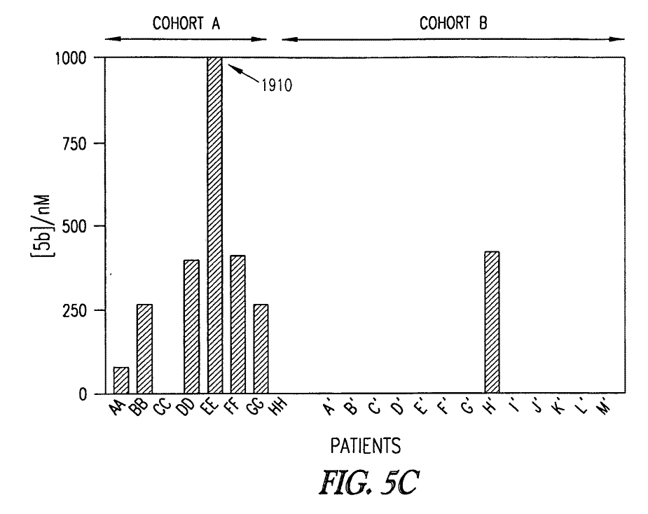

For example, in the bloodstream (plasma) of six of eight patients with atherosclerosis disease states that were sufficiently advanced to warrant endarterectomy the aldol 5a was detected in amounts ranging from 70-1690 nM (FIG. 5C). However, there was detectable 5a in only one of fifteen plasma samples from patients that were randomly selected from a group of patients attending a general medical clinic.

Moreover, according to the invention, ozonation products of cholesterol can oxidatively modify LDL, and/or apoprotein B₁₀₀ (apoB-100), the protein component of LDL. Treatment of LDL with the seco-ketoaldehyde 4a or the aldol adduct 5a can reduce the α-helical content and increase the random coil content of LDL and/or apoB-100, thereby altering the secondary structure of this complex. More significantly, the seco-ketoaldehyde 4a or the aldol adduct 5a can increase lipid uptake by macrophages and promote the formation of foam cells.

The invention provides methods for counteracting these negative effects of cholesterol ozonation products.

### Identifying Agents to Counteract Cholesterol Ozonation Product Activities

As illustrated herein, cholesterol ozonation products can alter the differentiation, expression patterns, or chemotaxis of key cells involved in the development of atherosclerosis. For example, cholesterol ozonation products 4a and 5a can recruit macrophages to atherosclerotic tissues, increase the differentiation of monocytes into macrophages, increase the expression a cell adhesion molecule (E-selectin) in endothelial cells and increase expression of Class A scavenger receptor (SR-A) in macrophages.

Therefore, one aspect of the invention is a method for identifying an agent that can inhibit cholesterol ozonolysis product activity in a mammalian cell. This method involves contacting a cell with a test agent and observing whether the cell's differentiation, expression patterns, or chemotaxis changes, wherein the cell is cultured with a cholesterol ozonolysis product. In some embodiments, the cholesterol ozonation product is 4a or 5a.

In one embodiment, the method involves contacting a macrophage with a test agent and observing whether expression of Class A scavenger receptor (SR-A) increases in the macrophage after exposing the macrophage to a cholesterol ozonolysis product. The SR-A receptor is a key cell surface receptor responsible for macrophage internalization of modified LDL and oxysterols. Such uptake of LDL and oxysterols by macrophages leads to form cell formation. Thus, an agent that can inhibit SR-A expression when the cell is exposed to cholesterol ozonolysis products can be used to slow or inhibit foam cell build-up in atherosclerotic tissues. In some embodiments, the cell is exposed to cholesterol ozonolysis products 4a or 5a in the presence of LDL.

Another method of the invention involves identifying an agent that can inhibit recruitment of macrophages to atherosclerotic tissues. As illustrated herein, cholesterol ozonolysis products are chemotactic agents that attract macrophages. Macrophage buildup is one indicator of atherosclerotic plaque buildup. Thus, agents that counteract the chemotaxis of macrophages in the presence of cholesterol ozonolysis products can be used to inhibit atherosclerotic plaque formation. This method involves contacting a macrophage with a test agent and observing whether the macrophage migrates toward a source of a cholesterol ozonolysis product. In some embodiments, cholesterol ozonolysis products 4a or 5a are used as the cholesterol ozonolysis product.

Another aspect of the invention is a method for identifying an agent that can inhibit atherosclerosis by inhibiting an increase in E-selectin expression. This method involves contacting an endothelial cell with a test agent and observing whether expression of E-selectin increases in the endothelial cell exposing the endothelial to a cholesterol ozonolysis product. In some embodiments, the cell is exposed to cholesterol ozonolysis products 4a or 5a in the presence of LDL.

Another aspect of the invention is a method for identifying an agent that can inhibit monocyte differentiation into macrophages. As shown herein, cholesterol ozonolysis products increase monocyte differentiation into macrophages, which can become foam cells as described above. This method therefore involves contacting a monocyte with a test agent and observing whether the monocyte differentiates into a macrophage, wherein the monocyte is cultured with cholesterol ozonolysis product. In some embodiments, the monocyte is cultures with cholesterol ozonolysis product 4a or 5a.

Control assays can be performed to help identify agents that counteract the effects of cholesterol ozonolysis products upon cell expression, cell recruitment and cell differentiation. Thus, a control assay can be performed where the assay is performed as described above but the cell is not exposed to or cultured in the presence of the test agent. The same procedure for assessing cellular recruitment, expression, and differentiation in the presence of the test agent can be used to observe and/or quantify levels of recruitment, expression and differentiation without the agent. The test agent is a useful anti-atherosclerosis agent when the test agent leads to significantly lower levels of cell recruitment, expression and differentiation compared to control levels observed without the test agent.

Additional control assays may also be performed. For example, in some embodiments it may be useful to perform an assay where the cell is not exposed to or cultured in the presence of a cholesterol ozonolysis product. Such a control assay permits assessment of cellular recruitment, expression, and differentiation without the influence of cholesterol ozonation products. The effect of a test agent upon cellular recruitment, expression, and differentiation alone can therefore be assessed to ascertain whether the test agent directly blocks or modulates the activity of cholesterol ozonation products or whether the agent independently modulates cellular recruitment, expression, and differentiation.

Cellular expression can be assessed and/or quantified by any available procedure. For example, cellular expression can be assessed by reverse transcriptase-polymerase chain reaction (RT-PCR), by northern analysis and other procedures.

Cellular recruitment can be assessed by available procedures for observing cell migration. For example, cellular recruitment can be assessed using a modified Boyden chamber migration assay. Zwirner et al. (1998) Eur. J. Immunol. 28: 1570-77; Wilkinson (1988) Methods Enzymol. 162: 38-50. These assays generally involve using two chambers separated by a membrane with a pore size that permits cell movement between the chambers (e.g., a 5 μm pore size). Cells to be tested are placed in a first chamber and the chemotactic agent (e.g. a cholesterol ozonation product) is placed in the other (second) chamber. The cells will migrate from the first to the second chamber through the membrane when the chemotactic agent is present in the second chamber. The number of cells that migrate toward the chemotactic agent is a measure of the level of cellular recruitment. Hence, if significantly fewer cells migrate toward the chemotactic agent (i.e. toward the cholesterol ozonolysis product) when the test agent is present, that test agent is an agent that can inhibit atherosclerosis by inhibiting macrophage migration to atherosclerotic lesions.

Cellular differentiation can be assessed by culturing monocytes in the presence of a test agent and a cholesterol ozonolysis product and observing whether morphological changes characteristic of differentiated macrophages are detected. Such macrophage morphological changes include cellular adherence, development of intracellular vesicles and long cytoplasmic extensions that are characteristic of differentiated macrophages. Alternatively, macrophage differentiation can be detected by observing cellular expression of macrophage markers. Activated macrophages express a 38 LD GPI-anchored folate receptor that binds folate and folate-derivatized compounds. Hence, expression of 38 LD GPI-anchored folate receptor on the cell or binding of folate or folate-derivatized compound can be used to detect macrophages.

Once a test agent is identified that can modulate cellular recruitment, expression, and/or differentiation, that test agent can be further tested in animal models of atherosclerosis to further characterize its efficacy, assess its toxicity and determine appropriate dosages.

### Methods for Counteracting the Effects of Cholesterol Ozonation Products

According to the invention, the negative effects of cholesterol ozonation products can be controlled or inhibited by agents that bind to or inhibit such cholesterol ozonation products. In other embodiments, cholesterol ozonation products can be used as markers and site-specific antigens for atherosclerotic lesions so that therapeutic agents can be delivered to atherosclerotic lesions.

The invention therefore relates to methods for treating or preventing a vascular condition, a circulatory condition involving deposit of cholesterol, and problems associated with release of cytotoxic cholesterol ozonation products. Such conditions and problems can be associated with loss, injury or disruption of the vasculature within an anatomical site or system. The term “vascular condition” or “vascular disease” refers to a state of vascular tissue where blood flow is, or can become, impaired.

Vascular diseases that can be treated or prevented by the present invention are vascular diseases of mammals. The word mammal means any mammal. Some examples of mammals include, for example, pet animals, such as dogs and cats; farm animals, such as pigs, cattle, sheep, and goats; laboratory animals, such as mice and rats; primates, such as monkeys, apes, and chimpanzees; and humans. In some embodiments, humans are preferably treated by the methods of the invention.

Examples of vascular conditions and diseases that can be treated or prevented with the compositions and methods of the invention include atherosclerosis (or arteriosclerosis), preeclampsia, peripheral vascular disease, heart disease, and stroke. Thus, the invention is directed to methods of treating diseases such as stroke, atherosclerosis, acute coronary syndromes including unstable angina, thrombosis and myocardial infarction, plaque rupture, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), stroke or thrombosis.

The methods and reagents provided herein can also be used at any stage of atherosclerotic plaque development. According to a new classification adopted by the American Heart Association and used for this study, eight lesion types can be distinguished during progression of atherosclerosis.

Type I lesions are formed by small lipid deposits (intracellular and in macrophage foam cells) in the intima and cause the initial and most minimal changes in the arterial wall. Such changes do not thicken the arterial wall.

Type II lesions are characterized by fatty streaks including yellow-colored streaks or patches that increase the thickness of the intima by less than a millimeter. They consist of accumulation of more lipid than is observed in type I lesions. The lipid content is approximately 20-25% of the dry weight of the lesion. Most of the lipid is intracellular, mainly in macrophage foam cells, and smooth muscle cells. The extracellular space may contain lipid droplets, but these are smaller than those within the cell, and small vesicular particles. These lipid droplets have previously been described as consisting of cholesterol esters (cholesteryl oleate and cholesteryl linoleate), cholesterol, and phospholipids. According to the invention, cholesterol ozonation products can promote lipid uptake by cells associated with atherosclerotic lesion formation. Moreover, cholesterol ozonation products like those described herein can accumulate intracellularly or extracellularly within such cells.

Type III lesions are also described as preatheroma lesions. In type III lesions the intima is thickened only slightly more than observed for type II lesions. Type III lesions do not obstruct arterial blood flow. The extracellular lipid and vesicular particles are identical to those found in type II lesions, but are present in increased amount (approx. 25-35% dry weight) and start to accumulate in small pools.

Type IV lesions are associated with atheroma. They are crescent-shaped and increase the thickness of the artery. The lesion may not narrow the arterial lumen much except for persons with very high plasma cholesterol levels (for many people, the lesion can not be visible by angiography). Type IV lesions consist of an extensive accumulation (approx. 60% dry weight) of extracellular lipid in the intimal layer (sometimes called a lipid core). The lipid core may contain small clamps of minerals. These lesions are susceptible to rupture and to formation of mural thrombi.

Type V lesions are associated with fibroatheroma. They have one or multiple layers of fibrous tissue consisting mainly of type I collagen. Type V lesions have increased wall thickness and, as the atherosclerosis progresses increased reduction of the lumen. These lesions have features that permit further subdivision. In type Va lesions, new tissue is part of a lesion with a lipid core. In type Vb lesions, the lipid core and other parts of the lesion are calcified (leading to Type VII lesions). In type Vc lesions, the lipid core is absent and lipid generally is minimal (leading to Type VIII lesions). Generally, the lesions that undergo disruption are type Va lesions. They are relatively soft and have a high concentration of cholesterol esters rather than free cholesterol monohydrate crystals. Type V lesions can rupture and form mural thrombi.

Type VI lesions are complicated lesions having disruptions of the lesion surface such as fissures, erosions or ulcerations (Type VIa), hematoma or hemorrhage (Type VIb), and thrombotic deposits (Type VIc) that are superimposed on Type IV and V lesions. Type VI lesions have increased lesion thickness and the lumen is often completely blocked. These lesions can convert to type V lesions, but they are larger and more obstructive.

Type VII lesions are calcified lesions characterized by large mineralization of the more advanced lesions. Mineralization takes the form of calcium phosphate and apatite, replacing the accumulated remnants of dead cells and extracellular lipid.

Type VIII lesions are fibrotic lesions consisting mainly of layers of collagen, with little lipid. Type VIII could be a consequence of lipid regression of a thrombus or of a lipidic lesion with an extension converted to collagen. These lesions may obstruct the lumen of medium-sized arteries.

While endothelial injury is believed to be an initial step in the formation of the atherosclerotic lesions, such injury often leads to cholesterol accumulation, intimal thickening, cellular proliferation, and formation of connective tissue fibers. IgG and complement factor C3 accumulation in injured endothelial cells and nonendothelialized intima has been observed. Mononuclear phagocytes derived from blood are also part of the cell population in atherosclerotic lesions.

According to the invention, accumulation of such antibodies and immune cells may lead to production of reactive oxygen species, which in turn can contribute to the formation of cholesterol ozonation products. As described above, lipid accumulation within cells associated with atherosclerotic lesion formation is one of the key steps in the development of problematic atherosclerotic lesions. One mechanism for plaque formation is that fatty deposits lead to an influx of macrophages, which in turn are followed by T cells, B cells, and antibody production. As shown herein, cholesterol ozonation products of the invention promote lipid uptake by macrophages and increase the formation of macrophage foam cells. Accordingly, the inventors have shown that cholesterol ozonation products can exacerbate inflammatory vascular diseases such as atherosclerosis.

The invention contemplates therapeutic compositions and methods for preventing and treating vascular diseases and conditions. Compositions provided the invention can be used to treat vascular conditions in a variety of ways.

In one embodiment, the invention provides a method that involves administering to the animal an antibody or binding agent that can bind to a cholesterol ozonation product. Such an antibody or binding entity modulates the cholesterol ozonation product and inhibits the lipid-loading and foam cell generating activity of such ozonation products. Preferably an antibody used in this method does not generate reactive oxygen species such as ozone. An antibody or binding agent can bind any of the cholesterol ozonation products described herein, for example, the seco-ketoaldehyde 4a, its aldol adduct 5a or the related compounds 6a-15a or 7c. These antibodies and binding entities can be produced using haptens that are structurally related to the cholesterol ozonation products and that generate antibody or binding entity preparations that cross-react with naturally produced cholesterol ozonation products.

For example, in another embodiment, the invention provides a hapten having any one of formulae 3c, 13a, 13b, 14a, 14b, 15a or 14b that can be used to generate antibodies that can react with the ozonation products of cholesterol:

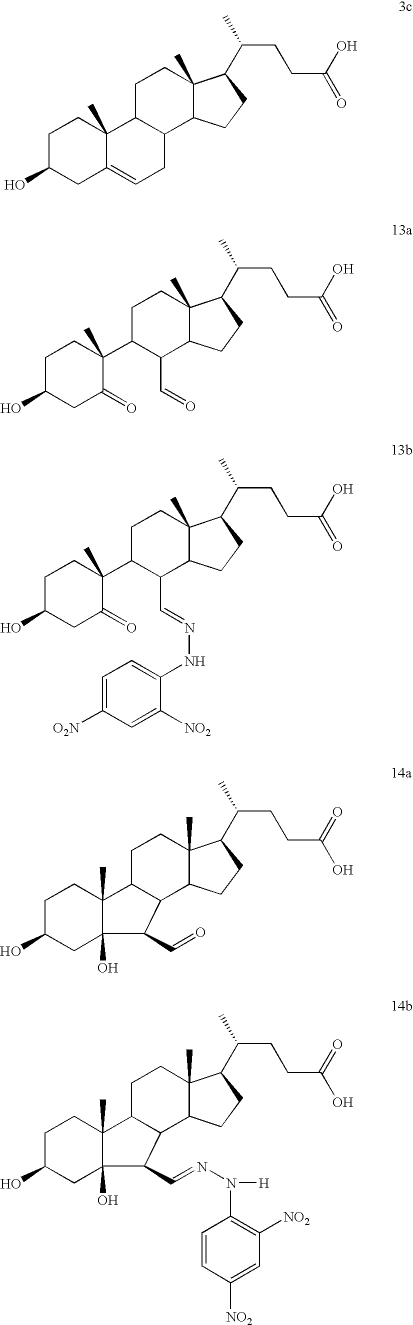

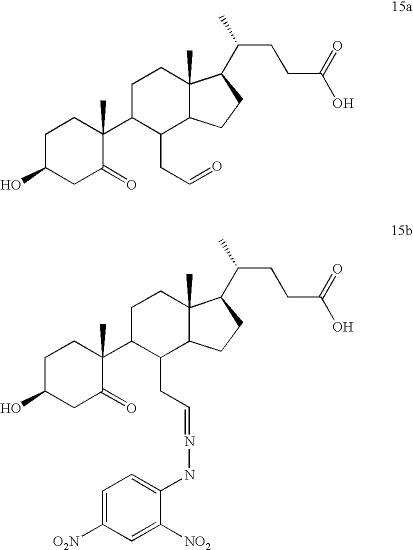

Hybridomas KA1-11C5 and KA1-7A6, raised against a compound having formula 15a, were deposited under the terms of the Budapest Treaty on Aug. 29, 2003 with the American Type Culture Collection (10801 University Blvd., Manassas, Va., 20110-2209 USA (ATCC)) as ATCC Accession No. ATCC Numbers PTA-5427 and PTA-5428. Hybridomas KA2-8F6 and KA2-1E9, raised against a compound having formula 14a, were deposited with the ATCC under the terms of the Budapest Treaty also on Aug. 29, 2003 as ATCC Accession No. ATCC PTA-5429 and PTA-5430.

In another embodiment, cholesterol ozonation products are used as targets or markers of atherosclerotic lesions. Thus, therapeutic agents linked to binding entities that are capable of binding to cholesterol ozonation products can be administered to a mammal suffering from atherosclerosis. To treat or prevent atherosclerosis and related vascular diseases, cholesterol ozonation products can therefore be used as targets or markers of atherosclerotic lesions. Any of the cholesterol ozonation products, for example, the seco-ketoaldehyde 4a, its aldol adduct 5a, and/or the A-ring dehydration product 6a can be used as a marker for targeting binding entities and/or therapeutic agents to atherosclerotic plaque. Alternatively, any of the cholesterol ozonation products having formulae 7a through 15a or 7c can be used as markers for targeting binding entities and/or therapeutic agents to atherosclerotic plaque.

The binding entity is designed not only to bind to the cholesterol ozonation product(s) but also to deliver a therapeutic agent or drug that can act locally to reduce the atherosclerotic lesion or prevent further occlusion of the artery. Alternatively, the therapeutic agent can block, shield, or inhibit the negative effects of a cholesterol ozonation product. Thus, therapeutic agents linked to binding entities that are capable of binding to cholesterol ozonation products can be administered to a mammal suffering from a vascular disease such as atherosclerosis.

Binding entities that can recognize cholesterol ozonation products and can be used in the methods of the invention include any small molecule, polypeptide or antibody capable of binding a cholesterol ozonation product. Such polypeptides and antibodies are described in further detail below.

Therapeutic agents that can be linked to such binding entities include any anti-oxidant, drug, factor, compound, peptide, polypeptide, nucleic acid or other agent that one of skill in the art would select for reducing oxidation or treating an atherosclerotic lesion. Any therapeutic agent that would counteract the activity of a cholesterol ozonation product or serve to dissolve, digest, break up or inhibit the growth of atherosclerotic plaque or otherwise ameliorate the progression of atherosclerosis could be used.

A therapeutic agent is also intended to comprise active metabolites and prodrugs thereof. An active metabolite is an active derivative of a therapeutic agent produced when the therapeutic agent is metabolized. A prodrug is a compound that is either metabolized to a therapeutic agent or is metabolized to an active metabolite(s) of a therapeutic agent. This invention can be used to administer therapeutic agents such as small molecular weight compounds, antioxidants, radionuclides, drugs, enzymes, peptides, proteins, nucleic acids that encode therapeutic polypeptides, expression vectors, anti-sense RNA, small interfering RNA, ribozymes, or antibodies.

For example, the binding entities of the invention can be used to deliver fibrinolytic agents. Such therapeutic agents include, for example, thrombolytic agents such as streptokinase, tissue plasminogen activator, plasmin and urokinase, anti-thrombotic agents such as tissue factor protease inhibitors (TFPI), anti-inflammatory agents, metalloproteinase inhibitors, nematode-extracted anticoagulant proteins (NAPs), drugs that inhibit cell growth, drugs that inhibit cell growth factors, and the like. Further examples of therapeutic agents that can be linked to the binding entities of the invention include the following:
1) Agents that and modulate lipid levels (for example, HMG-CoA reductase inhibitors, thyromimetics, fibrates, agonists of peroxisome proliferator-activated receptors (PPAR) (including PPAR-alpha, PPAR-gamma and/or PPAR-delta);
2) Agents that control and modulate oxidative processes, for example, anti-oxidants, modifiers of reactive oxygen species, modifiers of cholesterol ozonation products, or inhibitors of factors (including cholesterol ozonation products) that modify lipoproteins;
3) Agents that control and modulate insulin resistance and/or activity or glucose metabolism or activity including, but not limited to, agonists of PPAR-alpha, PPAR-gamma and/or PPAR-delta, modifiers of DPP-IV, and modifiers of glucocorticoid receptors;
4) Agents that control and modulate expression of receptors or adhesion molecules or integrins on endothelial cells or smooth muscle cells in any vascular location;
5) Agents that control and modulate the activity of endothelial cells or smooth muscle cells in any vascular location;
6) Agents that control and modulate inflammation associated receptors including, but not limited to chemokine receptors, RAGE, toll-like receptors, angiotensin receptors, TGF receptors, interleukin receptors, TNF receptors, C-reactive protein receptors, and other receptors involved in inflammatory signaling pathways including the activation of NF-kb;
7) Agents that control and modulate proliferation, apoptosis or necrosis of endothelial cells, vascular smooth muscle or lymphocytes, monocytes, and neutrophils adhering to or within the vessel;
8) Agents that control and modulate production, degradation, or cross-linking of any extracellular matrix proteins including, but not limited to, collagen, elastin, and proteoglycans;
9) Agents that control and modulate activation, secretion or lipid loading of any cell type within mammalian vessels;
10) Agents that control and modulate the activation, proliferation or any other modification of dendritic cells within mammalian vessels;
11) Agents that control and modulate the activation, adhesion, or other processes that modify platelet events at the level of the vessel wall;
12) Agents that control and modulate the production of ozone by antibodies and/or atherosclerotic plaque material; and
13) Anti-inflammatory agents such as ibuprofen, acetylsalicylic acid, ketoprofen and the like.

The binding entities of the invention can be covalently linked or otherwise associated with such therapeutic agents. Liposomes bearing the binding entities and containing the therapeutic agent(s) can be used to facilitate therapeutic delivery. Upon administration, the therapeutic agents will become localized at the site of atherosclerotic lesions by the binding entities and will help control, diminish or otherwise facilitate improved arterial blood flow in the region of the atherosclerotic lesion. The binding entities of the invention can also be used to deliver nanoparticles, such as vectors for gene therapies.

Therapeutic agents contemplated by the invention also include “antioxidants”, defined as any molecule that has an antagonist effect to an oxidant. An antioxidant so defined includes 1) inhibitors of ozone or reactive oxygen species generation by an antibody, 2) inhibitors of cholesterol ozonation products, and 3) inhibitors of the toxic effects caused by cholesterol ozonation products. Preferred antioxidants include those that inhibit the production of cholesterol ozonation products as well as neutralizing those already formed. The antioxidant effect can occur by any mechanism, including catalysis. Antioxidants as a category include reactive oxygen species scavengers, ozone scavengers, or free radical scavengers. Antioxidants may be of different types so they are available if and when they are needed. In view of the presence of oxygen throughout an aerobic organism, antioxidants may be available in different cellular, tissue, organ and extracellular compartments. The latter include extracellular fluid spaces, intraocular fluids, synovial fluid, cerebrospinal fluid, gastrointestinal secretions, interstitial fluid, blood and lymphatic fluid. Antioxidants can be provided by supplementing the diet, or by injection, intravenous administration and the like.

Examples of antioxidants that can be used include but are not limited to ascorbic acid, α-tocopherol, γ-glutamylcysteinylglycine, γ-glutamyl transpeptidase, α-lipoic acid, dihydrolipoate, acetyl-5-methoxytryptamine, flavones, flavonenes, flavanols, catalase, peroxidase, superoxide dismutase, metallothionein, and butylated hydroxytoluene.

In another embodiment, the binding entities provide a means for employing laser angioplasty ablation of atherosclerotic plaque. One or more of the binding entities of the invention can be conjugated to a dye whose absorption maximum corresponds to the maximum emission wavelength of the laser to be used for angioplasty. After administration, the binding entity with the dye binds to a cholesterol ozonation product in an atherosclerotic lesion but exhibits substantially no binding to normal tissues. The dyes can be used as a target for focusing laser energy on atherosclerotic lesions. During the ablation procedure, energy from the laser is absorbed by the dye and thus can be concentrated on the diseased areas. As a consequence, the efficiency of ablation would be increased while minimizing damage to surrounding normal tissues.

A wide variety of fluorescent dyes, are available for conjugation to binding entities. A number of methods for conjugating dyes to proteins, and in particular antibodies, have been published. The choice of dye and method of conjugation would be readily apparent to one skilled in the arts of laser angioplasty and protein chemistry. One dye that may be useful in laser angioplasty is rhodamine. Rhodamine is a fluorescent dye whose various derivatives absorb light at a wavelength of approximately 570 nm.

A binding entity can be linked to a dye such as rhodamine by available procedures. For example, the binding entity can be dialyzed against 50 mM sodium borate buffer, pH 8.2. A fresh solution of lissamine rhodamine B sulfonyl chloride (Molecular Probes, Inc. Eugene, Oreg.) can be prepared in dry acetone at 0.25 mg/mL. An aliquot of this solution representing a 6-fold molar excess of rhodamine over the amount of binding entity to be conjugated is transferred to a glass tube. The acetone is evaporated under a stream of dry argon. The dialyzed antibody is added to the rhodamine residue in the tube. The tube is capped, covered with aluminum foil, and incubated at 4° C. for 3 hours with constant shaking.

An aliquot of a 1.5M hydroxylamine hydrochloride (Sigma) solution (pH 8.0) equal to 1/10 the volume of the binding entity solution is added to the reaction mixture. This solution is incubated at 4° C. for 30 minutes with constant shaking. The reaction mixture is then dialyzed extensively against borate buffer in the dark. The rhodamine-antibody conjugate can be stored at 4° C. in the dark to avoid photo-bleaching of the dye.

After administration, the labeled binding entity specifically delivers the dye to atherosclerotic lesions and not to normal tissues. Tissues that bind the labeled binding entity can be ablated by application of laser a wavelength of approximately 570 nm.

In another embodiment, the binding entities of the invention can be used to deliver enzymes specifically to the site of an atherosclerotic lesion. The enzyme could be any enzyme capable of digesting one or more components of the plaque. The enzyme or a combination of enzymes would be conjugated to the binding entity by one of a variety of conjugation techniques known to one skilled in the art of protein chemistry.

In another approach, binding entities of the invention can be coupled to an inactive form of an enzyme, for example, a proenzyme or an enzyme-inhibitor complex. The advantage of this method would be that larger amounts of enzyme could be administered, thus delivering larger amounts of enzyme to the plaque while not causing any damage to normal tissues by the circulating conjugate. After the binding entity-enzyme conjugate has bound to the plaque and unbound circulating conjugate has cleared, the enzyme could be activated so as to begin digestion of the plaque. Activation would involve specific cleavage of the proenzyme or removal of an enzyme inhibitor.

In another embodiment, antibodies or binding entities that recognize and bind other factors in atherosclerotic lesions are used for delivery of therapeutic agents. A variety of soluble proteins have been extracted from human atherosclerotic plaque, including IgA, IgG, IgM, B1C(C3), α₁-antitrypsin, α₂-macroglobulin, fibrinogen, albumin, LDL, HDL, α₁-acid glycoprotein, β₂-glycoprotein, transferrin and ceruloplasmin. The diseased intima was also found to contain a small amount of tissue-bound IgG, IgA and B1C [Hollander, W. et al., Atherosclerosis, 34:391-405 (1979)]. IgG has been observed in lesions and adjacent endothelial tissue [Parums, D. et al., Atherosclerosis, 38:211-216 (1981), Hansson, G. et al., Experimental and Molecular Pathology, 34:264-280 (1981), Hannson, G. et al., Acta Path. Microbiol. Immunol. Scand. Sect. A., 92:429-435 (1984)]. Any of these proteins can be used for delivery of a therapeutic agent to atherosclerotic lesions.

U.S. Pat. No. 6,025,477 provides a purified antigen that is specifically present as an extracellular component of atherosclerotic plaque and antibodies directed against the antigen. This antigen has a complex carbohydrate structure, and a molecular weight greater than 200,000 daltons and being. The monoclonal antibody described by the hybridoma Q10E7 selectively binds to atherosclerotic lesions. U.S. Pat. No. 6,025,477 is incorporated herein by reference.

In a further embodiment, the cytotoxic ozonation products of cholesterol that are released endogenously into the bloodstream of patients suffering from atherosclerosis can be removed by in vivo treatment of the patient or ex vivo treatment of the patient's blood with a binding entity that binds the ozonation product(s) and facilitates removal of the cholesterol ozonation product. As described herein, plasma samples from atherosclerosis patients had detectable levels of cholesterol ozonation products. A test group of atherosclerosis patients included eight patients that had atherosclerosis disease states sufficiently advanced to warrant endarterectomy. A control group of patients was randomly selected from patients that had attended a general medical clinic. Six of the eight patients in the test group had detectable plasma levels of aldol 5a ranging in amounts from 70-1690 nM (see FIG. 5). In only one of the fifteen plasma samples from the control group was there detectable 5a. The ketoaldehyde 4a was not actually detected in any patient's blood sample but the assay employed had a detection limit of about 1-10 nM. It is possible that the ketoaldehyde 4a is converted into the aldol 5a during or after release from atherosclerotic lesions. Hence, in therapies designed to remove cytotoxic cholesterol ozonation products from the bloodstream of atherosclerosis patients, the aldol adduct 5a may be the primary product to remove.

Therapeutic methods provided by the invention for treating vascular conditions and removing cytotoxic cholesterol ozonation products from the bloodstream can avoid surgical and other invasive and dangerous treatment procedures. For example, current therapeutic methods for arteriosclerosis are generally divided into surgical methods and methods for medically managing the disease. Surgical methods may entail vascular graft procedures to bypass regions of occlusion (e.g., coronary artery bypass grafting), removal of occluding plaques from the arterial wall (e.g., carotid endarterectomy), or percutaneously cracking the plaques (e.g., balloon angioplasty). Surgical therapies carry significant risk and treat only individual lesions, one at a time. Surgical therapies also do not limit the progression of atherosclerosis and are associated with complications such as restenosis.

Targeting cholesterol ozonation products using the methods of the invention may simplify treatment of heart disease and permit patients to avoid the risks and complications of surgery. One of the reasons that the present methods may avoid surgery is that the cholesterol ozonation products identified herein appear to be specifically produced by atherosclerotic lesions. Hence, targeting those ozonation products will accurately and specifically target the sites and causes of atherosclerosis. Similarly, removal of cytotoxic ozonation products from the bloodstream can prevent further injury to the vascular system.

Identifying Agents that Prevent Ozonation of Cholesterol

The invention further provides methods for identifying agents that block formation of reactive oxygen species by antibodies. Such methods involve screening for agents that inhibit reactive oxygen species production by antibodies that have been provided with a source of singlet oxygen (¹O₂*). The singlet oxygen (¹O₂*) employed can be a natural source of singlet oxygen (¹O₂*) such as a neutrophil. Alternatively, the singlet oxygen (¹O₂*) can be a synthetic source of singlet oxygen. For example, “sensitizer” molecules such as metal-free porphyrin can be used that generate singlet oxygen after exposure to an inducer such as light.

As has been shown by the inventors, essentially any antibody or neutrophil can generate powerful reactive oxygen species, including but not limited to superoxide radical (O₂⁻), hydroxyl radical (OH.), hydrogen peroxide H₂O₂ or ozone (O₃) when the antibodies or neutrophils are exposed to singlet oxygen (¹O₂*). See P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003). Hence, as used herein the term “reactive oxygen species” means an antibody-generated oxygen species. These reactive oxygen species possess one or more unpaired electrons or are otherwise reactive because they are readily react with other molecules. Such reactive oxygen species include but are not limited to superoxide free radicals, hydrogen peroxide, hydroxyl radical, peroxyl radical, ozone and other short-lived trioxygen adducts that have the same chemical signature as ozone. Moreover, as illustrated by experimental work described herein, ozone is generated within atherosclerotic lesions.

Antibodies perform the conversion of singlet oxygen (¹O₂*) to reactive oxygen species without the need for any other component of the immune system, that is, without the need for the complement cascade or phagocytosis. The ability to produce reactive oxygen species from singlet oxygen is present in intact immunoglobulins and well as in antibody fragments such as Fab, F(ab′)₂ and Fv fragments. Also, the activity is not associated with the presence of disulfides in an antibody molecule. However, the ability of an antibody to generate a reactive oxygen species from singlet oxygen is abolished if the antibody is denatured. This indicates that an intact or substantially intact three dimensional structure is needed for generation of reactive oxygen species by an antibody.

The minimum requirement for generating reactive oxygen species by an antibody is the presence of oxygen. Thus, aerobic conditions are generally required. More specifically, use of antibodies in vivo is dependent on the availability of the key substrate ¹O₂* but, such ¹O₂* is produced during a variety of physiological events and is available in vivo. See J. F. Kanofsky Chem.-Biol. Interactions 70, 1 (1989) and references therein. For example, ¹O₂* is produced including reperfusion. X. Zhai and M. Ashraf Am. J. Physiol. 269 (Heart Circ. Physiol. 38) H1229 (1995). Also, ¹O₂* is produced in neutrophil activation during phagocytosis. J. R. Kanofsky, H. Hoogland, R. Wever, S. J. Weiss J. Biol. Chem. 263, 9692 (1988); Babior et al., Amer. J. Med., 109:33-34 (2000).

Singlet oxygen (¹O₂) also results from irradiation by light of metal-free porphyrin precursors. The biological conversion of singlet oxygen to reactive oxygen species occurs in light, including visible light, infrared light and under ultraviolet irradiation conditions. When visible light conditions are employed, the production of singlet oxygen can be enhanced using other molecules that can provide a source of singlet oxygen. Molecules that generate singlet oxygen include molecules that generate singlet oxygen without the need for other factors or inducers as well as “sensitizer” molecules that can generate singlet oxygen after exposure to an inducer. Examples of molecules that can generate singlet oxygen without the need for other factors or inducers include, but are not limited to, endoperoxides. In some embodiments, the endoperoxide employed can be an anthracene-9,10-dipropionic acid endoperoxide. Examples of sensitizer molecules also include, but are not limited to, pterins, flavins, hematoporphyrins, tetrakis(4-sulfonatophenyl)porphyrin, bipyridyl ruthenium(II) complexes, rose Bengal dyes, quinones, rhodamine dyes, phthalocyanines, hypocrellins, rubrocyanins, pinacyanols or allocyanines.

Sensitizer molecules can be induced to generate singlet oxygen when exposed to an inducer. One such inducer is light. Such light can be visible light, ultraviolet light, or infrared light, depending upon the type and structure of the sensitizer.

Accordingly, the invention provides a method for screening for an agent that can modulate production of reactive oxygen species by an antibody that involves contacting a mixture of an antibody and a singlet oxygen source with an agent and observing whether reactive oxygen production by the antibody is modulated. In some embodiments, the agent preferably produces less reactive oxygen species. In other embodiments, the agent preferably produces more reactive oxygen species.

### Uses for Cytotoxic Cholesterol Ozonation Products

As provided herein, the seco-ketoaldehyde 4a, its aldol adduct 5a and the related compounds 6a-15a and 7c are cytotoxic to a number of cell types. The structure of compound 7c is shown below.

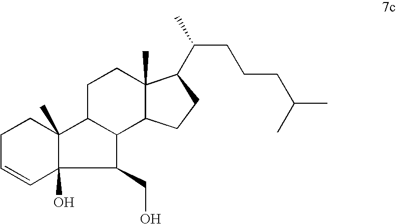

For example, as illustrated herein the seco-ketoaldehyde 4a and its aldol adduct 5a are cytotoxic towards a human B-lymphocyte (WI-L2) described in Levy et al., Cancer 22, 517 (1968); a T-lymphocyte cell line (Jurkat E6.1) described in Weiss et al., J. Immunol. 133, 123 (1984); a vascular smooth muscle cell line (VSMC) and an abdominal aorta endothelial (HAEC) cell line described in Folkman et al., Proc. Natl. Acad. Sci. U.S.A. 76, 5217 (1979); a murine tissue macrophage (J774A.1) described in Ralph et al., J. Exp. Med. 143, 1528 (1976); and an alveolar macrophage cell line (MH-S) described in Mbawuike et al., J. Leukoc. Biol. 46, 119 (1989).

Using similar procedures, compounds 6a, 7a, 7c, 10a, 11a and 12a have been shown by the inventors to be cytotoxic to leukocyte cell lines and the seco-ketoaldehyde 4a and its aldol adduct 5a have been shown to be cytotoxic towards neuronal cell lines.

The invention therefore provides compositions containing the present cholesterol ozonation products and methods for treating and preventing inappropriate immune responses, autoimmune diseases, cancer, tumors, bacterial infections, viral infections, fungal infections, ulcers and/or other conditions or diseases where localized administration of a cytotoxin is beneficial.

The cytotoxin may have to be masked so that cholesterol ozonation will not adversely affect non-diseased tissues. One example of a procedure for masking the 4a or 5a cytotoxins in the formulation includes the use of liposomes. For example, the 4a or 5a cytotoxins can be placed within liposomes and a binding entity can be anchored within the phospholipid membrane of the liposome. The binding entity facilitates localization of the liposomes to the diseased tissue, and the lipid coat of the liposomes protects non-diseased tissues from the cytotoxic cholesterol ozonation products. The liposomal lipid coat can also interact with the lipids in the atherosclerotic lesions, thereby leading to fusion and release of the liposomal contents.

### Treating Cancers and Tumors

In another embodiment, the cytotoxic cholesterol ozonation products can be used to treat or prevent cancer. The invention thus provides anti-cancer cytotoxins that include any of compounds 4a through 15a and 7c, and pharmaceutical compositions thereof. As illustrated herein, the 4a, 5a and related compounds are cytotoxic against a number of mammalian cells including a human B-lymphocyte (WI-L2) described in Levy et al., Cancer 22, 517 (1968); a T-lymphocyte cell line (Jurkat E6.1) described in Weiss et al., J. Immunol. 133, 123 (1984); a vascular smooth muscle cell line (VSMC) and an abdominal aorta endothelial (HAEC) cell line described in Folkman et al., Proc. Natl. Acad. Sci. U.S.A. 76, 5217 (1979); a murine tissue macrophage (J774A.1) described in Ralph et al., J. Exp. Med. 143, 1528 (1976); and an alveolar macrophage cell line (MH-S) described in Mbawuike et al., J. Leukoc. Biol. 46, 119 (1989). Hence, the 4a and 5a cytotoxins can be used to kill or inhibit the growth of a number of different cancerous cell types.

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc. Both human and veterinary uses are contemplated.

As used herein the terms “normal mammalian cell” and “normal animal cell” are defined as a cell that is growing under normal growth control mechanisms (e.g., genetic control) and displays normal cellular differentiation. Cancer cells differ from normal cells in their growth patterns and in the nature of their cell surfaces. For example cancer cells tend to grow continuously and chaotically, without regard for their neighbors, among other characteristics well known in the art.

Mammals and other animals including birds may be treated by the methods and compositions described and claimed herein. Such mammals and birds include humans, dogs, cats, and livestock, for example, horses, cattle, sheep, goats, chickens, turkeys and the like.

The invention therefore provides a pharmaceutical composition for treating, inhibiting or preventing growth of a cancer cell in an animal comprising a cytotoxin including a compound of any one of compounds 4a through 15a and 7c, in an amount effective to treat or prevent a target cancer in the animal, and a pharmaceutically acceptable carrier, wherein the cytotoxin can be linked to an antibody or binding entity that selectively binds to the cancer cell.

The invention also provides a method for treating, inhibiting or preventing growth of a cancer cell in an animal comprising contacting a target cancer cell with a cytotoxin including a compound of any one of compounds 4a through 15a and 7c, in an amount sufficient to induce target cancer cell death without inducing an undesirable amount of non-cancerous mammalian cell death, wherein the cytotoxin can be linked to an antibody or binding entity that selectively binds to the cancer cell.

The invention further provides a method for treating, inhibiting or preventing growth of a cancer cell in an animal comprising administering a formulation comprising a cytotoxin including a compound of any one of compounds 4a through 15a and 7c, in an amount sufficient to induce target cancer cell death or inhibit cancer cell growth without inducing an undesirable amount of non-cancerous mammalian cell death, wherein the cytotoxin can be linked to an antibody or binding entity that selectively binds to the cancer cell.

The antibody or binding entity that selectively binds to the cancer cell can recognize or bind to any available tissue-specific antigen or cancer marker selected by one of skill in the art.

Tumor antigens and antibodies against tumor antigens are known. Binding entities, antibodies or antibody fragments reactive with a tumor associated antigens present on carcinoma or sarcoma cells or lymphomas are disclosed, for example, in Goldenberg et al., Journal of Clinical Oncology, Vol 9, No. 4, pp. 548-564, 1991 and Pawlak et al., Cancer Research, Vol 49, pp. 4568-4577, 1989, as LL-2 and EPB-2 (same). Others are disclosed in Primus et al. Cancer Res., 43:686-692, 1983, which discloses anti-CEA monoclonal antibodies; Hansen et al. Proc. Am. Assoc. Cancer Res., 30:414, 1989, which discloses and compares anti-CEA monoclonal antibodies; Gold et al. Cancer Res., 50:6405-6409, 1990, which disclose monoclonal antibodies reactive with colon-specific antigen-p (CSAP) and Gold et al. Proc. Am. Assoc, Cancer Res., 31:292, 1990, which disclose a monoclonal antibody reactive with a pancreatic, tumor-associated epitope. The KC-4 murine monoclonal antibody can also be used; it is specific to a unique antigenic determinant, and selectivity binds strongly to neoplastic carcinoma cells and not to normal human tissue (U.S. Pat. No. 4,708,930 to Coulter).

The BrE-3 antibody (Peterson et al., Hybridoma 9:221 (1990); U.S. Pat. No. 5,075,219) was shown to bind to the tandem repeat of the polypeptide core of human breast epithelial mucin. When the mucin is deglycosylated, the presence of more tandem repeat epitopes is exposed and the binding of the antibody increases. Thus, antibodies such as BrE-3 bind preferentially to neoplastic carcinoma tumors because these express an unglycosylated form of the breast epithelial mucin that is not expressed in normal epithelial tissue. The preferential binding combined with an observed low concentration of epitope for these antibodies in the circulation of carcinoma patients, such as breast cancer patients, makes antibodies having specificity for a mucin epitope highly effective for cancer therapy.

Hence, the invention provides compositions and methods for treating and/or preventing cancer.

Treating Transplant Rejection

T-lymphocytes are the cell type primarily responsible for causing rejection of allografts (e.g., transplanted organs such as the heart). T-lymphocytes (killer and helper) respond to allografts by undergoing a proliferative burst characterized by the transitory presence on the T-lymphocyte surfaces of IL-2 receptors. Killing these cells by the administration, during the proliferative burst, of a cytotoxin that reacts specifically with T-lymphocytes can inhibit allograft rejection. By linking a cytotoxin to a binding entity that specifically recognizes activated T-lymphocytes, the cytotoxin will advantageously fail to adversely affect other cells (including resting or long-term memory T-lymphocytes needed for fighting infections). One cell surface protein that is present on activated T-lymphocytes, but not on resting or long-term memory T-lymphocytes is the interleukin-2 (IL-2) receptor. Hence, use of a cytotoxin linked to a binding entity that binds an IL-2 receptor provides selectivity for activated T-lymphocytes.

As described herein the cytotoxin employed is the seco-ketoaldehyde 4a, its aldol adduct 5a or any of the related compounds having compounds 4a through 15a and 7c. These cholesterol ozonation products are cytotoxic towards a T-lymphocyte cell line (Jurkat E6.1) described in Weiss et al., J. Immunol. 133, 123 (1984). In some embodiments the 4a-12a or 7c cytotoxin can induce cell lysis, induce cell death or inhibit cell growth.

Because the 4a-14a or 7c cytotoxin inhibits the functioning or growth of T-lymphocytes, the binding entity employed can bind so that it blocks or does not block IL-2 interaction with the IL-2 receptor. However, blocking the site to which IL-2 binds would provide further assurance that the T-lymphocyte will not be fully activated and can result in several important phenomena which contribute to inhibition of tissue rejection.

By selectively targeting activated T-lymphocytes, the methods of the invention inhibit allograft rejection in a manner which does not cause general immune suppression, with its resulting risk of life-threatening infections. In addition, the method spares donor-specific T suppressor cells, which can thus proliferate and aid in prolonging allograft survival. Moreover, therapy need not be continuous following the allograft, but can be discontinued after a course of treatment.

One embodiment of the invention employs, as the IL-2 receptor-specific binding entity, for example, an antibody that is specific for the IL-2 receptor on T-lymphocytes, covalently linked to a 4a-15a or 7c cytotoxin. The cytotoxin can lyse T-lymphocytes to which the binding entity binds. Antibodies specific for the IL-2 receptor on T-lymphocytes can be made using standard techniques as described herein. Alternatively, such antibodies can be purchased, for example, from Becton Dickenson Company (e.g., mouse-human monoclonal anti-IL-2 receptor antibodies). The antibody can be monoclonal or polyclonal, and can be derived from any suitable animal. Where the antibody is monoclonal and the mammal being treated is human, human or humanized anti-IL-2 receptor antibodies are preferred.

Production and initial screening of monoclonal antibodies to yield those specific for the IL-2 receptor can be carried out as described in Uchiyama et al. (1981) J. Immunol. 126 (4), 1393. Briefly, this method employs the following steps. Human cultured T-lymphocytes are injected into mammals, e.g., mice, and the spleens of the immunized animals are removed and the spleen cells separated and then fused with immortal cells, e.g., mouse or human myeloma cells, to form hybridomas. The antibody-containing supernatants from the cultured supernatants are then screened for those specific for the IL-2 receptor, using a complement-dependent cytotoxicity test, as follows. Human T-lymphocytes and EBV transformed B-lymphocytes are labeled with ⁵¹Cr sodium chromate and used as target cells; these cells are incubated with hybridoma culture supernatants and with complement, and then the supernatants are collected and counted with a gamma counter. Those supernatants exhibiting toxicity against activated T-lymphocytes, but not resting T- or B-lymphocytes, are selected, and then subjected to a further screening step to select those supernatants containing antibody that precipitates (i.e., is specifically reactive with) the 50 kilodalton glycoprotein IL-2 receptor (described in detail in Leonard et al. (1983) P.N.A.S. USA 80, 6957). The desired anti-IL-2 receptor antibody is purified from the supernatants using conventional methods.

Treatment of Autoimmune Diseases

The CD4⁺ T-lymphocyte (herein referred to as the CD4⁺ T-cell) is the central player in the immune system because of the “help” it provides to other leukocytes in fighting off infection and potential cancerous cells. CD4⁺ T-cells play essential roles in both humeral and cell-mediated immunity. Additionally they act during parasite infection to promote the differentiation of eosinophils and mast cells. If the CD4⁺ T-cell population is depleted (as is the case in AIDS patients) the host is rendered susceptible to a number of pathogens and tumors that do not ordinarily pose a threat to the host.

However, while CD4⁺ T-cells play an important beneficial role in disease prevention, the aberrant function of these cells can produce serious problems. In some individuals, the aberrant function of CD4⁺ T-cells leads to autoimmunity and to other diseases. Autoimmune diseases in which CD4⁺ T-cells have been implicated include multiple sclerosis, rheumatoid arthritis and autoimmune uveitis. In essence these diseases involve an aberrant immune response in which the immune system is subverted from its normal role of attacking invading pathogens and instead attacks host body tissues, leading to illness and even death. The targeted host tissues attacked are different for different autoimmune diseases. For example, in multiple sclerosis the immune system attacks the white matter of the brain and spinal cord, and in rheumatoid arthritis the immune system attacks the synovial lining of the joints. Activated CD4⁺ T-cells have also been implicated in other illnesses, including rejection of transplant tissues and organs and development of CD4⁺ T-cell lymphomas.

This invention therefore provides a method of treatment useful for undesired immune responses. In one embodiment, the invention provides method for treating or preventing T-cell mediated autoimmune diseases. In other embodiments, the invention provides methods for treating and preventing activated CD4⁺ T-cell mediated autoimmune diseases. Diseases that can be treated include, for example, multiple sclerosis, rheumatoid arthritis, sarcoidosis and autoimmune uveitis, graft versus host disease (GVHD) and/or inflammatory bowel disease.

The cytotoxin employed in these methods is the seco-ketoaldehyde 4a, its aldol adduct 5a or a compound having any of formulae 4a through 15a or 7c. These cholesterol ozonation products are cytotoxic towards a T-lymphocyte cell line (Jurkat E6.1) described in Weiss et al., J. Immunol. 133, 123 (1984). In some embodiments the 4a-15a or 7c cytotoxin can induce cell lysis, induce cell death or inhibit cell growth.

The 4a-15a or 7c cytotoxins are utilized in conjunction with a binding entity that specifically recognizes and binds to T-cells or, preferably, to CD4⁺ T-cells. Such a binding entity can be any binding entity having selectivity for T-cells. For example, any T-cell specific antigen can be used to generate antibodies that can act as binding entities for delivery of the cytotoxic cholesterol ozonation products provided herein. Examples include the human receptor protein H4-1 BB. A cDNA for H4-1 BB encoded in the vector pH4-1 BB was deposited at the Agricultural Research Service Culture Collection and assigned the accession number: NRRL B21131. Antibodies specific for this H4-1BB protein are described in U.S. Pat. No. 6,569,997.

According to U.S. Pat. No. 6,566,082, a particular protein antigen, termed OX-40, is specifically expressed on the cell surface of antigen activated T-cells especially, for example, activated CD4⁺ T-cells. Using the EAE disease model in rats, this antigen was shown to be expressed on the surface of activated autoantigen-specific CD4⁺ T-cells present at the site of inflammation (the spinal cord in this disease model) but absent on CD4⁺ T-cells at non-inflammatory sites. The highest expression of this antigen on these CD4⁺ T-cells was found to occur on the day prior to initiation of clinical signs of autoimmunity; and the expression of this antigen decreased as the disease progressed. The specificity of expression of the OX-40 antigen and the transient nature of this expression, shown for the first time in the present invention, motivated the testing of this antigen as a possible target for antibody mediated depletion of activated T-cells in animals such as humans with T-cell mediated conditions.

It has been shown that CD4⁺ T-cells are responsible for several experimentally induced autoimmune diseases in animals, including experimental autoimmune endephalomyelitis (EAE), collagen induced arthritis (CIA), and experimental autoimmune uveitis (EAU). Such animal models can be used for testing the methods and formulations provided herein.

### Treatment of Ulcers

Helicobacter pylori is a curved, microaerophilic, gram negative bacterium that was isolated for the first time in 1982 from stomach biopsies of patients with chronic gastritis, Warren et al., Lancet:1273-75 (1983). Originally named Campylobacter pylori, it has been recognized to be part of a separate genus named Helicobacter, Goodwin et al., Int. J. Syst. Bacteriol. 39:397-405 (1989). The bacterium colonizes the human gastric mucosa, and infection can persist for decades. During the last few years, the presence of the bacterium has been associated with chronic gastritis type B, a condition that may remain asymptomatic in most infected persons but increases considerably the risk of peptic ulcer and gastric adenocarcinoma. Other studies strongly suggest that H. pylori infection may be either a cause or a cofactor of type B gastritis, peptic ulcers, and gastric tumors, see e.g., Blaser, Gastroenterology 93:371-83 (1987); Dooley et al., New Engl. J. Med. 321:1562-66 (1989); Parsonnet et al., New Engl. J. Med. 325:1127-31 (1991). H. pylori is believed to be transmitted by the oral route, Thomas et al., Lancet:340, 1194 (1992). The risk of infection increases with age, Graham et al., Gastroenterology 100:1495-1501 (1991), and is facilitated by crowding, Drumm et al., New Engl. J. Med. 4322:359-63 (1990); Blaser, Clin. Infect. Dis. 15:386-93 (1992). In developed countries, the presence of antibodies against H. pylori antigens increases from less than 20% to over 50% in people 30 and 60 years old respectively, Jones et al., Med. Microbiol. 22:57-62 (1986); Morris et al., N. Z. Med. J. 99:657-59 (1986), while in developing countries over 80% of the population are already infected by the age of twenty, Graham et al., Digestive Diseases and Sciences 36:1084-88 (1991).

According to the invention, a cytotoxin linked to a binding entity that binds to an H. pylori antigen can be used to inhibit H. pylori growth. The cytotoxin employed is the seco-ketoaldehyde 4a, its aldol adduct 5a or a compound having any one of formulae 6a through 15a or 7c. In some embodiments the 4a or 15a or 7c cytotoxin can induce cell lysis, induce cell death or inhibit cell growth.

Treatment of Microbial Infections

The cytotoxic cholesterol ozonation products of the invention can also be used to modulate the growth and infection of microbes.

Infections of the following target microbial organisms can be treated by the cytotoxic cholesterol ozonation products of the invention: Aeromonas spp., Bacillus spp., Bacteroides spp., Campylobacter spp., Clostridium spp., Enterobacter spp., Enterococcus spp., Escherichia spp., Gastrospirillum sp., Helicobacter spp., Klebsiella spp., Salmonella spp., Shigella spp., Staphylococcus spp., Pseudomonas spp., Vibrio spp., Yersinia spp., and the like. Infections that can be treated by the cytotoxic cholesterol ozonation products of the invention include those associated with staph infections (Staphylococcus aureus), typhus (Salmonella typhi), food poisoning (Escherichia coli, such as O157:H7), bascillary dysentery (Shigella dysenteria), pneumonia (Psuedomonas aerugenosa and/or Pseudomonas cepacia), cholera (Vivrio cholerae), ulcers (Helicobacter pylori) and others. E. coli serotype 0157:H7 has been implicated in the pathogenesis of diarrhea, hemorrhagic colitis, hemolytic uremic syndrome (HUS) and thrombotic thrombocytopenic purpura (TTP). The cytotoxic cholesterol ozonation products of the invention are also active against drug-resistant and multiply-drug resistant strains of bacteria, for example, multiply-resistant strains of Staphylococcus aureus and vancomycin-resistant strains of Enterococcus faecium and Enterococcus faecalis.

The anti-microbial compositions of the invention are also effective against viruses. The term “virus” refers to DNA and RNA viruses, viroids, and prions. Viruses include both enveloped and non-enveloped viruses, for example, hepatitis A virus, hepatitis B virus, hepatitis C virus, human immunodeficiency virus (HIV), poxviruses, herpes viruses, adenoviruses, papovaviruses, parvoviruses, reoviruses, orbiviruses, picornaviruses, rotaviruses, alphaviruses, rubivirues, influenza virus type A and B, flaviviruses, coronaviruses, paramyxoviruses, morbilliviruses, pneumoviruses, rhabdoviruses, lyssaviruses, orthmyxoviruses, bunyaviruses, phleboviruses, nairoviruses, hepadnaviruses, arenaviruses, retroviruses, enteroviruses, rhinoviruses and the filovirus.

The compounds of the present invention are active antifungal agents useful in treating fungal infections in animals, including humans, for the treatment of systemic, topical and mucosal infections. Examples of fungal infections that can be treated by the present invention include infections by Candida, Aspergillus, and Fusarium. In some embodiments the fungal infection is caused by Candida albicans or Candida glabrata.

Compounds of the invention are useful for the treatment of variety of fungal infections in animals, including humans. Such infections include superficial, cutaneous, subcutaneous and systemic mycotic infections such as respiratory tract infections, gastrointestinal tract infections, cardiovascular infections, urinary tract infections, CNS infections, candidiasis and chronic muccocandidiasis and skin infections caused by fungi, cutaneous and mucocutaneous candidiasis, athletes foot, paronychia, fungal nappy rash, candida vulvitis, candida balanitis and otitis externa. They may be used as prophylactic agents to prevent systemic and topical fungal infections. Use as prophylactic agents may be appropriate as part of a selective gut decontamination regimen in the prevention of infection in immunocompromised patients, e.g. AIDS patients, patients receiving cancer therapy or transplant patients.

Several species of Aspergillus are known to cause invasive sinopulmonary infections in seriously immunocompromised patients. Following inhalation of spores, clinical aspergillosis can occur in three major presentations. The first presentation, allergic bronchopulmonary aspergillosis, develops when Aspergillus species colonize the bronchial tree and release antigens that cause a hypersensitivity pneumonitis. The second presentation, aspergilloma or “fungus ball,” develops in pulmonary cavities, often in concert with other lung diseases such as tuberculosis. The third form, invasive pulmonary or disseminated aspergillosis, is a life threatening infection with a high mortality rate.

Anti-microbial activity of the cytotoxic cholesterol ozonation products can be evaluated against these varieties of microbes using methods available to one of skill in the art. Anti-microbial activity, for example, is determined by identifying the minimum inhibitory concentration (MIC) of a cytotoxic cholesterol ozonation product of the present invention that prevents growth of a particular microbial species. In one embodiment, anti-microbial activity is the amount of cytotoxic cholesterol ozonation product that kills 50% of the microbes when measured using standard dose or dose response methods.

Methods of evaluating therapeutically effective dosages for treating a microbial infection with cytotoxic cholesterol ozonation products described herein include determining the minimum inhibitory concentration of a cytotoxic cholesterol ozonation product at which substantially no microbes grow in vitro. Such a method permits calculation of the approximate amount of cytotoxic cholesterol ozonation product needed per volume to inhibit microbial growth or to kill 50% of the microbes. Such amounts can be determined, for example, by standard microdilution methods. For example, a series of microbial culture tubes containing the same volume of medium and the substantially the same amount of microbes are prepared, and an aliquot of cytotoxic cholesterol ozonation product is added. The aliquots contain differing amounts of cytotoxic cholesterol ozonation product in the same volume of solution. The microbes are cultured for a period of time corresponding to one to ten generations and the number of microbes in the culture medium is determined.

The optical density of the cultural medium can also be used to estimate whether microbial growth has occurred—if no significant increase in optical density has occurred, then no significant microbial growth has occurred. However, if the optical density increases, then microbial growth has occurred. To determine how many microbial cells remain alive after exposure to a cytotoxic cholesterol ozonation product, a small aliquot of the culture medium can be removed at the time when the cytotoxic cholesterol ozonation product is added (time zero) and then at regular intervals thereafter. The aliquot of culture medium is spread onto a microbial culture plate, the plate is incubated under conditions conducive to microbial growth and, when colonies appear, the number of those colonies is counted.

### Antibodies and Binding Entities

The invention provides antibodies and binding entities that can bind cholesterol ozonation products or any target antigen that can act as a marker for delivery of the present cytotoxic ozonation products to sites of disease. As described herein antibodies and binding agents directed against cholesterol ozonation products can be used to inhibit or modulate the cytotoxicity of these cholesterol ozonation products and thereby treat vascular diseases such as atherosclerosis, heart disease, or cardiovascular disease. As also described above the cytotoxic cholesterol ozonation products can be linked to antibodies or binding agents and used for treating or preventing conditions and diseases such as autoimmune diseases, cancer, tumors, bacterial infections, viral infections, fungal infections, ulcers and/or other conditions or diseases where localized administration of a cytotoxin is beneficial.

As used herein, the term binding entities includes antibodies and other polypeptides capable of binding cholesterol ozonation products or other markers of disease.

Hence, in one embodiment, the invention provides antibody preparations and binding entities directed against cholesterol ozonation products, for example, the seco-ketoaldehyde 4a, its aldol adduct 5a, related compounds such as any of the 3c, 6a-15a or 7c cholesterol ozonation products or haptens. Such antibodies and binding entities are useful for treating cholesterol-related vascular diseases such as inflammatory vascular diseases, atherosclerosis, heart disease, and cardiovascular disease. In some embodiments, the cholesterol ozonation products can be chemically modified to facilitate preparation of antibodies. For example, hydrazone derivatives of the seco-ketoaldehyde 4a, its aldol adduct 5a and related compounds like any of compounds 3c, 6a-15a or 7c may be used for antibody preparation. These hydrozone derivatives include compounds having structures like those of compounds 4b, 4e, 5b, and any of 6b-15b or 10c.

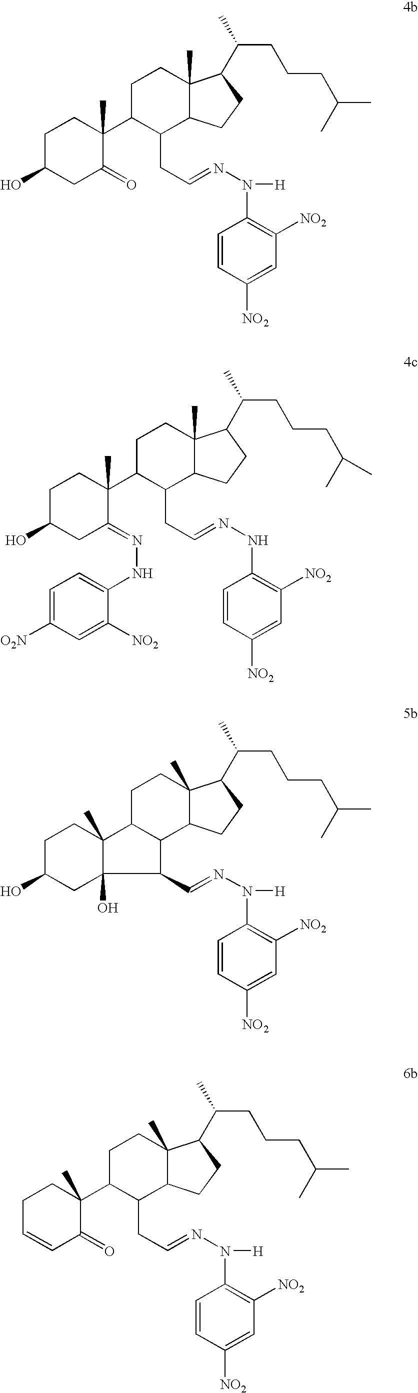

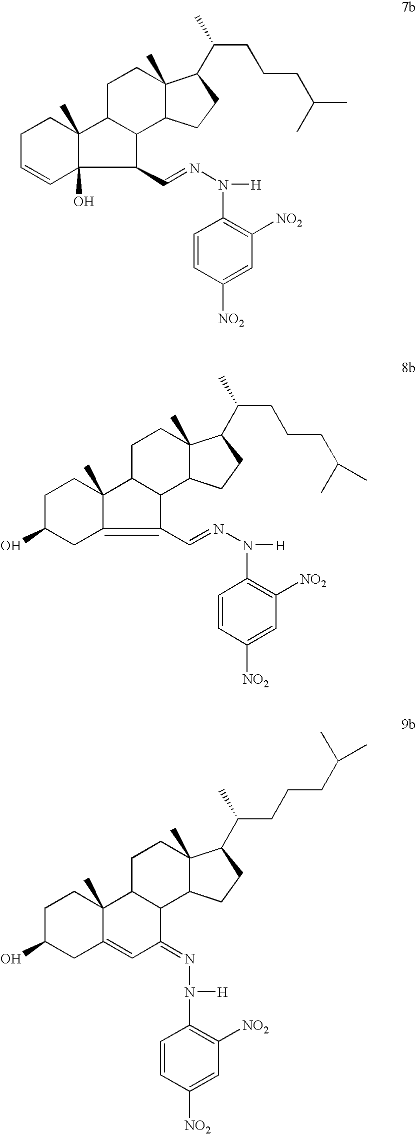

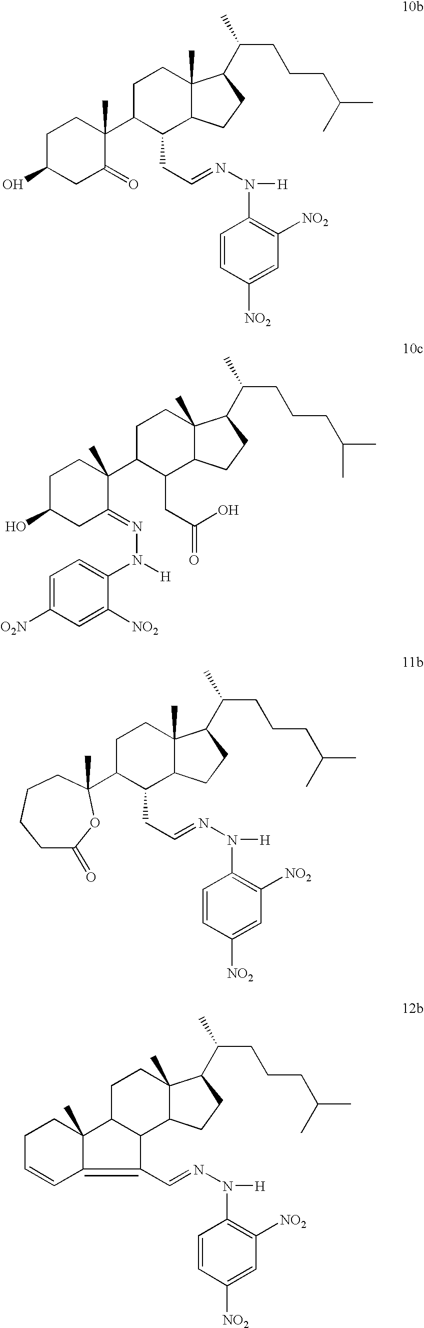

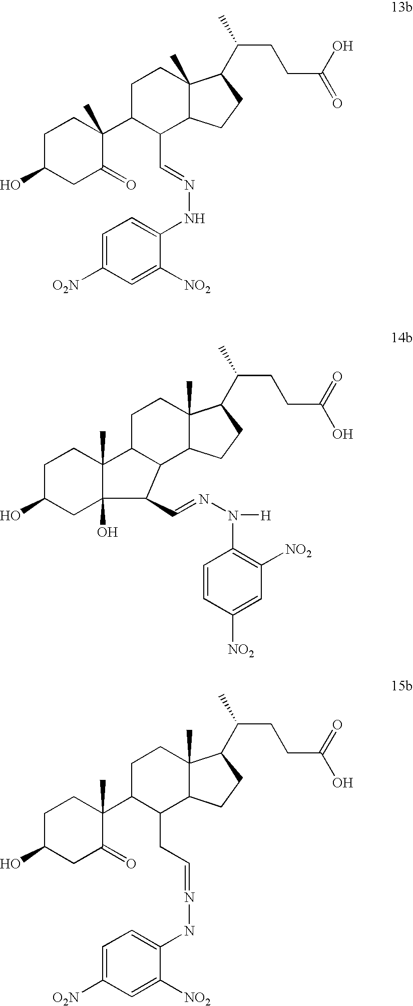

Cholesterol ozonation products can be converted to hydrozone derivatives, for example, by reaction with a hydrazine compound such as 2,4-dinitrophenyl hydrazine. In some embodiments, the reaction is carried out in an organic solvent such as acetonitrile, or alcohol (e.g. methanol or ethanol). An acidic environment and a non-oxygen containing, non-reactive atmosphere are often utilized.

The invention is further directed against haptens that are structurally related to the cholesterol ozonation products and the hydrazone derivatives of such ozonation products. For example, the invention provides a hapten having formula 3c, 13a, 13b, 14a, 14b, 15a or 15b that can be used to generate antibodies that can react with the ozonation and hydrazone products of cholesterol:

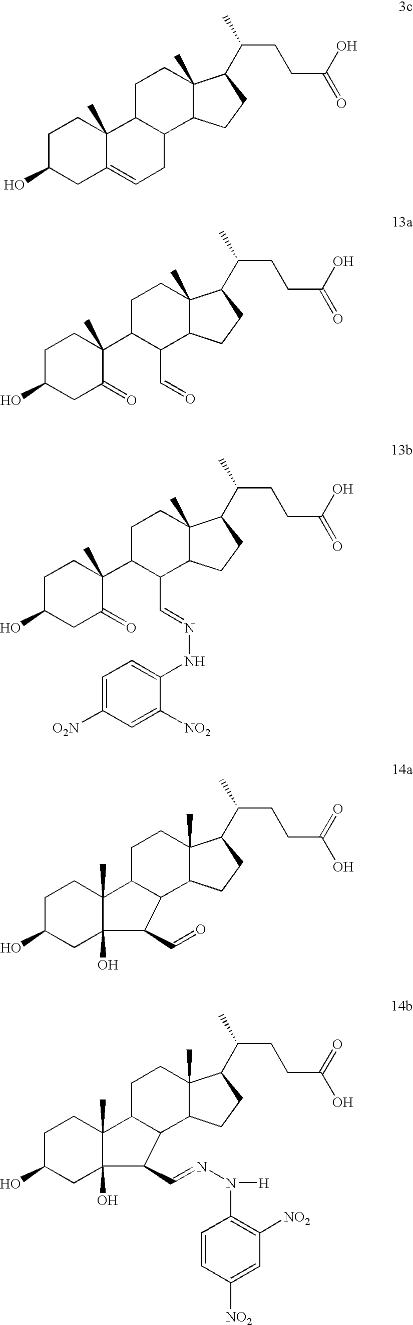

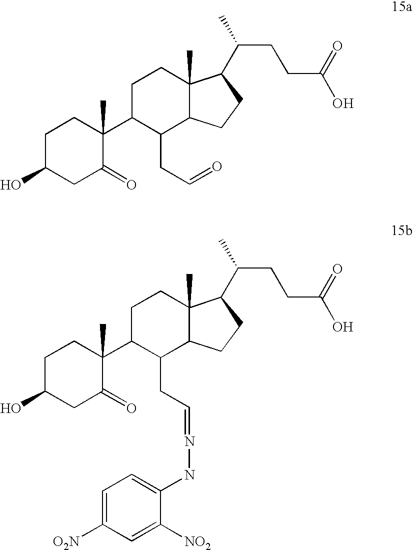

Hybridomas KA1-11C5 and KA1-7A6, raised against a compound having formula 15a, were deposited under the terms of the Budapest Treaty on Aug. 29, 2003 with the American Type Culture Collection (10801 University Blvd., Manassas, Va., 20110-2209 USA (ATCC)) as ATCC Accession No. ATCC Numbers PTA-5427 and PTA-5428. Hybridomas KA2-8F6 and KA2-1E9, raised against a compound having formula 14a, were deposited with the ATCC under the terms of the Budapest Treaty also on Aug. 29, 2003 as ATCC Accession No. ATCC PTA-5429 and PTA-5430.

The invention also provides antibodies and binding entities made by available procedures that can bind an ozonation product of cholesterol or any convenient marker of a disease. The binding domains of such antibodies, for example, the CDR regions of these antibodies, can also be transferred into or utilized with any convenient binding entity backbone.

Antibody molecules belong to a family of plasma proteins called immunoglobulins, whose basic building block, the immunoglobulin fold or domain, is used in various forms in many molecules of the immune system and other biological recognition systems. A standard antibody is a tetrameric structure consisting of two identical immunoglobulin heavy chains and two identical light chains and has a molecular weight of about 150,000 daltons.

The heavy and light chains of an antibody consist of different domains. Each light chain has one variable domain (VL) and one constant domain (CL), while each heavy chain has one variable domain (VH) and three or four constant domains (CH). See, e.g., Alzari, P. N., Lascombe, M.-B. & Poljak, R. J. (1988) Three-dimensional structure of antibodies. Annu. Rev. Immunol. 6, 555-580. Each domain, consisting of about 110 amino acid residues, is folded into a characteristic β-sandwich structure formed from two β-sheets packed against each other, the immunoglobulin fold. The VH and VL domains each have three complementarity determining regions (CDR)-3) that are loops, or turns, connecting β-strands at one end of the domains. The variable regions of both the light and heavy chains generally contribute to antigen specificity, although the contribution of the individual chains to specificity is not always equal. Antibody molecules have evolved to bind to a large number of molecules by using six randomized loops (CDRs).

Immunoglobulins can be assigned to different classes depending on the amino acid sequences of the constant domain of their heavy chains. There are at least five (5) major classes of immunoglobulins: IgA, IgD, IgE, IgG and IgM. Several of these may be further divided into subclasses (isotypes), for example, IgG-1, IgG-2, IgG-3 and IgG-4; IgA-1 and IgA-2. The heavy chain constant domains that correspond to the IgA, IgD, IgE, IgG and IgM classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of variable domains differ extensively in sequence from one antibody to the next. The variable domains are for binding and determine the specificity of each particular antibody for its particular antigen. However, the variability is not evenly distributed through the variable domains of antibodies. Instead, the variability is concentrated in three segments called complementarity determining regions (CDRs), also known as hypervariable regions in both the light chain and the heavy chain variable domains.

The more highly conserved portions of variable domains are called framework (FR) regions. The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from another chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific cholesterol ozonation product or derivative thereof.

Moreover, the binding regions, or CDR, of antibodies can be placed within the backbone of any convenient binding entity polypeptide. In preferred embodiments, in the context of methods described herein, an antibody, binding entity or fragment thereof is used that is immunospecific for any of compounds of formulae 3, 3c, 4a-15a, 7c as well as the derivatives thereof, including the hydrazone derivatives.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called Fab fragments, each with a single antigen binding site, and a residual Fc fragment. Fab fragments thus have an intact light chain and a portion of one heavy chain. Pepsin treatment yields an F(ab′)₂ fragment that has two antigen binding fragments that are capable of cross-linking antigen, and a residual fragment that is termed a pFc′ fragment. Fab′ fragments are obtained after reduction of a pepsin digested antibody, and consist of an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. As used herein, “functional fragment” with respect to antibodies, refers to Fv, F(ab) and F(ab′)₂ fragments.

Additional fragments can include diabodies, linear antibodies, single-chain antibody molecules, and multispecific antibodies formed from antibody fragments. Single chain antibodies are genetically engineered molecules containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, where the fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

Antibody fragments contemplated by the invention are therefore not full-length antibodies. However, such antibody fragments can have similar or improved immunological properties relative to a full-length antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment of the invention can have any upper size limit so long as it is has similar or improved immunological properties relative to an antibody that binds with specificity to a disease marker, for example, an ozonation product of cholesterol. For example, smaller binding entities and light chain antibody fragments can have less than about 200 amino acids, less than about 175 amino acids, less than about 150 amino acids, or less than about 120 amino acids if the antibody fragment is related to a light chain antibody subunit.

Moreover, larger binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibodies directed against disease markers can be made by any available procedure. Methods for the preparation of polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Monoclonal antibodies can also be employed in the invention. The term “monoclonal antibody” as used herein refers to an antibody obtained from a population of substantially homogeneous antibodies. In other words, the individual antibodies comprising the population are identical except for occasional naturally occurring mutations in some antibodies that may be present in minor amounts. Monoclonal antibodies are highly specific, being directed against a single antigenic site. Furthermore, in contrast to polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Fragments of such antibodies can also be used, so long as they exhibit the desired biological activity. See U.S. Pat. No. 4,816,567; Morrison et al. Proc. Natl. Acad. Sci. 81, 6851-55 (1984).

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of antibodies are available to those skilled in the art. For example, the monoclonal antibodies to be used in accordance with the present invention may be made by the hybridoma method as described above or may be made by recombinant methods, e.g., as described in U.S. Pat. No. 4,816,567. Monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression of nucleic acids encoding the antibody fragment in a suitable host. Antibody fragments can be obtained by pepsin or papain digestion of whole antibodies conventional methods. For example, antibody fragments can be produced by enzymatic cleavage of antibodies with pepsin to provide a 5S fragment described as F(ab′)₂. This fragment can be further cleaved using a thiol reducing agent, and optionally using a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated by reference in their entireties.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-25 (1986); Reichmann et al., Nature 332, 323-29 (1988); Presta, Curr. Op. Struct. Biol. 2, 593-596 (1992); Holmes, et al., J. Immunol. 158:2192-2201 (1997) and Vaswani, et al., Annals Allergy, Asthma & Immunol. 81:105-115 (1998).

While standardized procedures are available to generate antibodies, the size of antibodies, the multi-stranded structure of antibodies and the complexity of six binding loops present in antibodies constitute a hurdle to the improvement and the manufacture of large quantities of antibodies. Hence, the invention further contemplates using binding entities, which comprise polypeptides that can recognize and bind to disease markers, including ozonation products of cholesterol.

A number of proteins can serve as protein scaffolds to which binding domains for disease markers can be attached and thereby form a suitable binding entity. The binding domains bind or interact with the cholesterol ozonation products of the invention while the protein scaffold merely holds and stabilizes the binding domains so that they can bind. A number of protein scaffolds can be used. For example, phage capsid proteins can be used. See Review in Clackson & Wells, Trends Biotechnol. 12:173-84 (1994). Phage capsid proteins have been used as scaffolds for displaying random peptide sequences, including bovine pancreatic trypsin inhibitor (Roberts et al., PNAS 89:2429-33 (1992)), human growth hormone (Lowman et al., Biochemistry 30:10832-38 (1991)), Venturini et al., Protein Peptide Letters 1:70-75 (1994)), and the IgG binding domain of Streptococcus (O'Neil et al., Techniques in Protein Chemistry V (Crabb, L., ed.) pp. 517-24, Academic Press, San Diego (1994)). These scaffolds have displayed a single randomized loop or region that can be modified to include binding domains for disease markers such as cholesterol ozonation products.

Researchers have also used the small 74 amino acid α-amylase inhibitor Tendamistat as a presentation scaffold on the filamentous phage M13. McConnell, S. J., & Hoess, R. H., J. Mol. Biol. 250:460-470 (1995). Tendamistat is a α-sheet protein from Streptomyces tendae. It has a number of features that make it an attractive scaffold for binding peptides, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. The loops of Tendamistat can serve a similar function to the CDR loops found in immunoglobulins and can be easily randomized by in vitro mutagenesis. Tendamistat is derived from Streptomyces tendae and may be antigenic in humans. Hence, binding entities that employ Tendamistat are preferably employed in vitro.

Fibronectin type III domain has also been used as a protein scaffold to which binding entities can be attached. Fibronectin type III is part of a large subfamily (Fn3 family or s-type Ig family) of the immunoglobulin superfamily. Sequences, vectors and cloning procedures for using such a fibronectin type III domain as a protein scaffold for binding entities (e.g. CDR peptides) are provided, for example, in U.S. Patent Application Publication 20020019517. See also, Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994).

In the immune system, specific antibodies are selected and amplified from a large library (affinity maturation). The combinatorial techniques employed in immune cells can be mimicked by mutagenesis and generation of combinatorial libraries of binding entities. Variant binding entities, antibody fragments and antibodies therefore can also be generated through display-type technologies. Such display-type technologies include, for example, phage display, retroviral display, ribosomal display, and other techniques. Techniques available in the art can be used for generating libraries of binding entities, for screening those libraries and the selected binding entities can be subjected to additional maturation, such as affinity maturation. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743.

The invention therefore also provides methods of mutating antibodies, CDRs or binding domains to optimize their affinity, selectivity, binding strength and/or other desirable properties. A mutant binding domain refers to an amino acid sequence variant of a selected binding domain (e.g. a CDR). In general, one or more of the amino acid residues in the mutant binding domain is different from what is present in the reference binding domain. Such mutant antibodies necessarily have less than 100% sequence identity or similarity with the reference amino acid sequence. In general, mutant binding domains have at least 75% amino acid sequence identity or similarity with the amino acid sequence of the reference binding domain. Preferably, mutant binding domains have at least 80%, more preferably at least 85%, even more preferably at least 90%, and most preferably at least 95% amino acid sequence identity or similarity with the amino acid sequence of the reference binding domain.

For example, affinity maturation using phage display can be utilized as one method for generating mutant binding domains. Affinity maturation using phage display refers to a process described in Lowman et al., Biochemistry 30(45): 10832-10838 (1991), see also Hawkins et al., J. Mol Biol. 254: 889-896 (1992). While not strictly limited to the following description, this process can be described briefly as involving mutation of several binding domains or antibody hypervariable regions at a number of different sites with the goal of generating all possible amino acid substitutions at each site. The binding domain mutants thus generated are displayed in a monovalent fashion from filamentous phage particles as fusion proteins. Fusions are generally made to the gene III product of M13. The phage expressing the various mutants can be cycled through several rounds of selection for the trait of interest, e.g. binding affinity or selectivity. The mutants of interest are isolated and sequenced. Such methods are described in more detail in U.S. Pat. No. 5,750,373, U.S. Pat. No. 6,290,957 and Cunningham, B. C. et al., EMBO J. 13(11), 2508-2515 (1994).

Therefore, in one embodiment, the invention provides methods of manipulating binding entity or antibody polypeptides or the nucleic acids encoding them to generate binding entities, antibodies and antibody fragments with improved binding properties that recognize disease markers such as cholesterol ozonation products.

Such methods of mutating portions of an existing binding entity or antibody involve fusing a nucleic acid encoding a polypeptide that encodes a binding domain for a disease marker to a nucleic acid encoding a phage coat protein to generate a recombinant nucleic acid encoding a fusion protein, mutating the recombinant nucleic acid encoding the fusion protein to generate a mutant nucleic acid-encoding a mutant fusion protein, expressing the mutant fusion protein on the surface of a phage, and selecting phage that bind to a disease marker.

Accordingly, the invention provides antibodies, antibody fragments, and binding entity polypeptides that can recognize and bind to a disease marker (e.g., a cholesterol ozonation product, hapten or cholesterol derivative). The invention further provides methods of manipulating those antibodies, antibody fragments, and binding entity polypeptides to optimize their binding properties or other desirable properties (e.g., stability, size, ease of use).

### Dosages, Formulations and Routes of Administration

The compositions of the invention are administered so as to achieve a reduction in at least one symptom associated with a disease such as atherosclerosis, heart disease, cardiovascular disease, autoimmune diseases, cancer, tumors, bacterial infections, viral infections, fungal infections, ulcers and/or other conditions or diseases where localized administration of a cytotoxin is beneficial.

To achieve the desired effect(s), the cytotoxin, binding entity, antibody or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, whether the therapeutic agent is a cytotoxin, binding entity or antibody, the disease, the weight, the physical condition, the health, the age of the mammal, whether prevention or treatment is to be achieved, and if the therapeutic agent is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the cytotoxin(s), binding entities, antibodies or combinations thereof may be essentially continuous over a preselected period of time or may be in a series of spaced doses. Both local and systemic administration is contemplated.

To prepare the composition, the cytotoxin(s), binding entities, antibodies or combinations thereof are synthesized or otherwise obtained, and purified as necessary or desired. These therapeutic agents can then be lyophilized or stabilized, their concentrations can be adjusted to an appropriate amount, and the therapeutic agents can optionally be combined with other agents. The absolute weight of a given cytotoxin, binding entity, antibody or combination thereof that is included in a unit dose can vary widely. For example, about 0.01 to about 2 g, or about 0.1 to about 500 mg, of at least one cytotoxin, binding entity, or antibody specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the cytotoxin(s), binding entities, antibodies or combinations thereof can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic agents of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are generally combined with a pharmaceutically acceptable carrier, diluent or excipient to form a pharmaceutical formulation, or unit dosage form. For oral administration, the therapeutic agents may be present as a powder, a granular formulation, a solution, a suspension, an emulsion or in a natural or synthetic polymer or resin for ingestion of the active ingredients from a chewing gum. The therapeutic agents may also be presented as a bolus, electuary or paste. Orally administered therapeutic agents of the invention can also be formulated for sustained release. For example, the therapeutic agents can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic agents of the invention can be prepared by procedures known in the art using well-known and readily available ingredients. For example, the therapeutic agent can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethylene glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the therapeutic agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one therapeutic agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more of the therapeutic agents of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active agents and other ingredients may form suspensions, solutions, or emulsions in oily or aqueous vehicles, and may contain formulatory agents such as suspending, stabilizing and/or dispersing agents. Alternatively, the therapeutic agents and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Additionally, the therapeutic agents are well suited to formulation as sustained release dosage forms and the like. The formulations can be so constituted that they release the active agent, for example, in a particular part of the vascular system or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the therapeutic agents can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The active ingredients of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific immune response, vascular condition or disease. Any statistically significant attenuation of one or more symptoms of an immune response, vascular condition or disease that has been treated pursuant to the method of the present invention is considered to be a treatment of such immune response, vascular condition or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic agents of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the therapeutic agents of the present invention specific for the indication or disease to be treated. Dry aerosol in the form of finely divided solid therapeutic agent that are not dissolved or suspended in a liquid are also useful in the practice of the present invention. Therapeutic agents of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular immune response, vascular condition or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, bronchodilators and the like, whether for the conditions described or some other condition.

### Kits

The present invention further pertains to a packaged pharmaceutical composition such as a kit or other container for controlling, preventing or treating a disease. The kit or container holds a therapeutically effective amount of a pharmaceutical composition for controlling disease and instructions for using the pharmaceutical composition for control of the disease. The pharmaceutical composition includes at least one binding entity or antibody of the present invention, in a therapeutically effective amount such that the disease is controlled, prevented or treated.

In one embodiment, the kit comprises a container containing an antibody that specifically binds to an ozonation product of cholesterol. The antibody can have a directly attached or indirectly associated therapeutic agent. The antibody can also be provided in liquid form, powder form or other form permitting ready administration to an animal.

In another embodiment, the invention provides a pharmaceutical composition that includes at least one cytotoxic cholesterol ozonation product, in a therapeutically effective amount such that the disease is controlled, prevented or treated. Such a kit with an ozonation product of cholesterol that can be used, for example, as a cytotoxin for inhibiting or killing undesirable cell types.

In another embodiment of the present invention, the kit would contain a binding entity conjugated with a cytotoxic ozonation product of cholesterol. Such a kit could be used to treat patients suffering from autoimmune diseases, cancer, tumors, bacterial infections, viral infections, ulcers and/or other diseases where localized administration of a cytotoxin is beneficial. This binding entity-cytotoxin conjugate would preferably be provided in a form suitable for administration to a patient by injection. Thus, the kit might contain the binding entity-cytotoxin conjugate in a suspended form, such as suspended in a suitable pharmaceutical excipient. Alternatively, the conjugate could be in a solid form suitable for reconstitution.

The kits of the invention can also comprise containers with tools useful for administering the compositions of the invention. Such tools include syringes, swabs, catheters, antiseptic solutions and the like.

The following examples are illustrative of the present invention, but are not limiting. Numerous variations and modifications on the invention as set forth can be effected without departing from the spirit and scope of the present invention.

### Example 1

### Materials and Methods

Operative isolation and handling of atherosclerotic artery specimens. Tissue samples were obtained by carotid endarterectomy. The samples contained atherosclerotic plaque and some adherent intima and media. The protocol for plaque analysis was approved by the Scripps Clinic Human Subjects Committee and patient consent was obtained prior to surgery. Fresh carotid endarterectomy tissue was analyzed within 30 min of operative removal. Note that the plaque samples were neither stored nor preserved. All analytical manipulations were complete within 2 h of surgical removal. No fixatives were added to the specimens.

Oxidation of indigo carmine 1 by human atherosclerotic artery specimens. Endarterectomy specimens (n=15), isolated as described above, were divided into two sections of approximately equal wet weight (±5%). Each specimen was placed into phosphate buffered saline (PBS, pH 7.4, 1.8 mL) containing indigo carmine 1 (200 μM, Aldrich) and bovine catalase (100 μg). Indigo carmine 1 was added to act as a chemical trap for ozone. Takeuchi et al., Anal. Chim. Acta 230, 183 (1990); Takeuchi et al., Anal. Chem. 61, 619 (1989). Phorbal myristate (PMA, 40 μg in 0.2 mL of DMSO) or DMSO (0.2 mL) was added as an activator of protein kinase C. Each sample was homogenized using a tissue homogenizer for 10 min and then centrifuged (10,000 rpm for 10 min). The supernatants were decanted, passed through a filter (0.2 μm) and the filtrate was analyzed for the presence of isatin sulfonic acid 2 using quantitative HPLC.

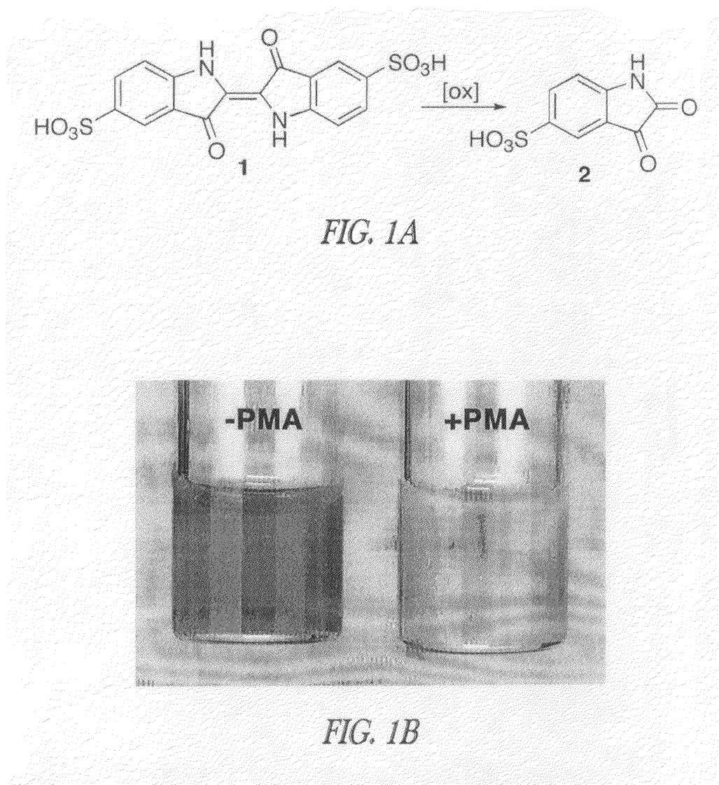

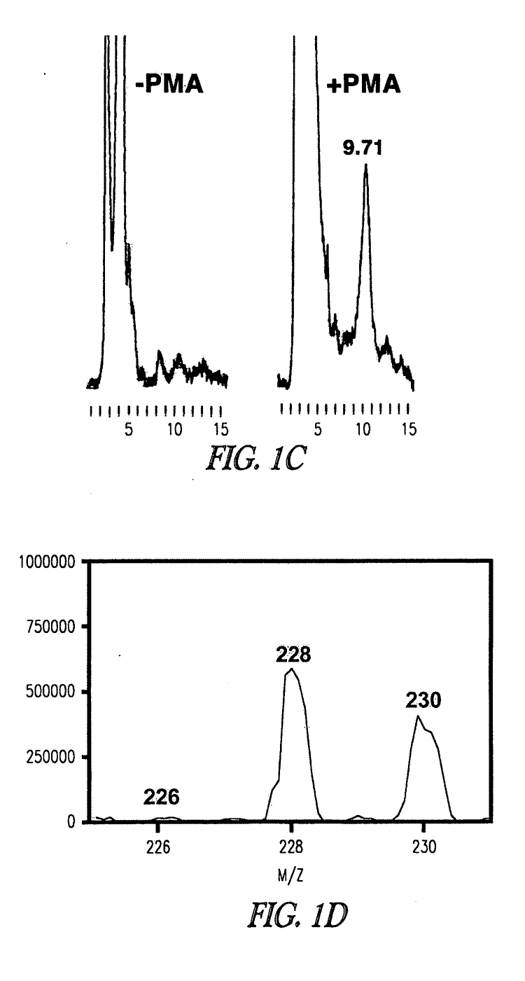

As shown by FIG. 1B, the visible absorbance of indigo carmine 1 was bleached and the reaction gave rise to a new chemical species that was detected using quantitative HPLC (Table 1), and that was identified as isatin sulfonic acid 2 (see also FIG. 1A).

HPLC assay for quantification of isatin sulfonic acid 2. HPLC analysis was performed on a Hitachi D-7000 machine, with an L-7200 autosampler, an L-7100 pump and an L-7400 u.v. detector (254 nm). The L-7100 was controlled using Hitachi-HSM software on a Dell GX150 PC computer. LC conditions were a Spherisorb RP-C₁₈ column and acetonitrile:water (0.1% TFA) (80:20) mobile phase at 1.2 mL/min. Isatin sulfonic acid 2 had a retention time, R_{T}, Of about 9.4 min. Quantification was performed by comparison of peak areas to standard curves of peak area vs. concentration of authentic samples using GraphPad v3.0 software for Macintosh (Table 1).

TABLE 1
Isatin sulfonic acid 2 (ISA) within activated
atherosclerotic artery material.

| Sample | ISA nmol/mg |
|---|---|
| 1 | 27.3 |
| 2 | 54.4 |
| 3 | 27.6 |
| 4 | 1.0 |
| 5 | 30.1 |
| 6 | 238.3 |
| 7 | 39.4 |
| 8 | 152.9 |
| 9 | 127 |
| 10 | 262.1 |
| 11 | 27.9 |
| 12 | 64.6 |
| 13 | 1.4 |
| 14 | 3.2 |
| 15 | 32.1 |

Mean ± SEM = 72.62 ± 21.69

Oxidation of indigo carmine 1 by human atherosclerotic artery specimens in H₂¹⁸O. This experiment was conducted as described in the indigo carmine assay above with the following exceptions. First, each plaque specimen (n=2) was added to phosphate buffer (10 mM, pH 7.4) in greater than 95% H₂¹⁸O. Second, the filtrate was desalted on a PD10 column and analyzed by negative electrospray mass spectrometry on a Finnegan electrospray mass spectrometer. The raw ion abundance data was extracted into Graphpad Prism v 3.0 format for presentation.

These experiments indicate that in the presence of plaque material and H₂¹⁸O (>95% ¹⁸O), the ¹⁸O isotope is incorporated into the lactam carbonyl of isatin sulfonic acid 2. Because only ozone could oxidatively cleave the double bond of indigo carmine 1 and promote isotope incorporation into the lactam carbonyl of isatin sulfonic acid 2 from H₂¹⁸O, ozone was likely the reactive oxygen species that oxidized indigo carmine 1. Hence, ozone is generated within atherosclerotic lesions. See also, P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003).

Extraction and derivatization procedure of aldehydes from atheromatous artery specimens. Endarterectomy specimens isolated as described above were divided into two sections of approximately equal wet weight (±5%). Each specimen was placed into phosphate buffered saline (PBS, pH 7.4, 1.8 mL) containing bovine catalase (100 μg) and either phorbal myristate (40 μg in 0.2 mL of DMSO) or DMSO (0.2 mL). Each sample was homogenized using a tissue homogenizer for 10 min. The homogenized endarterectomy samples, isolated as described above, were then washed with dichloromethane (DCM, 3×5 mL). The combined organic fractions were evaporated in vacuo. The residue was dissolved in ethanol (0.9 mL) and a solution of 2,4-dinitrophenyl hydrazine (100 μL, 2 mM, and 1N HCl) in ethanol was added. Nitrogen was bubbled through the solution for 5 min and then the solution was stirred for 2 h. The resultant suspension was filtered through a 0.22 μm filter and the filtrate was analyzed by the HPLC assay vide infra. When cholesterol 3 (1-20 μM) was treated under these conditions, no 4a or 5a was formed. The amount of 4b detected in atheromatous artery extracts both prior to and after PMA addition was subjected to a student two tail t-test analysis to determine the significance of PMA-addition on 4a levels in the artery extracts (p<0.05 was considered to be significant) and was determined with Graphpad v3.0 software for Macintosh.

During the derivatization of 4a under these conditions, about 20% of 4a was converted into 5b over a range of 4a concentrations (5 to 100 μM). These data indicate that a measured amount of 5a, exceeding 20% of the 4a present in the same plaque samples, arose from ozonolysis of 3 followed by aldolization. The extent of conversion of 4a into 6b under the employed derivatization conditions was consistently <2% over a range of 4a concentrations (5 to 100 μM). These observations indicate that the amount of 6a present within the plaque extracts that exceeds 2% of the amount of ketoaldehyde 4a, was present prior to derivatization and has arisen from the ozonolysis product 4a by β-elimination of water.

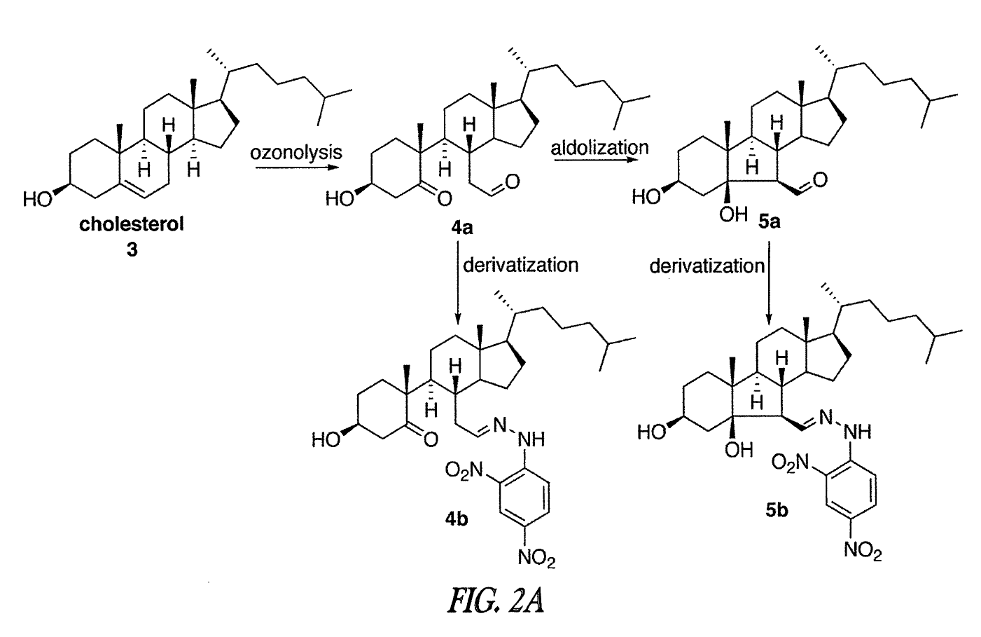

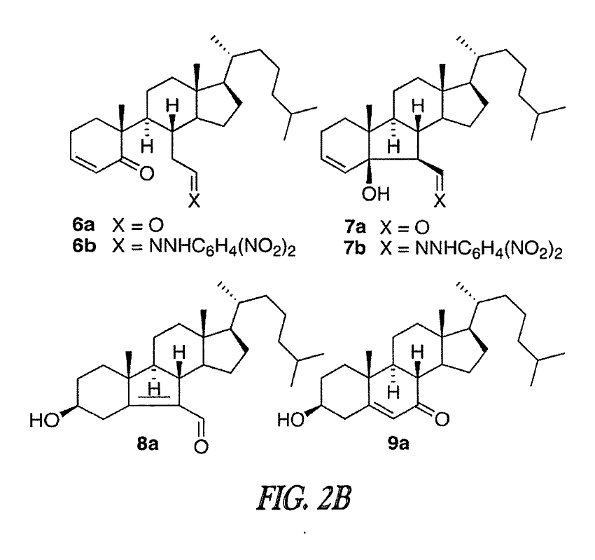

In addition to the three major hydrazone products 4b-6b, the hydrazone derivative of 7a (called 7b) was detected in trace amounts (<5 pmol/mg) in several plaque extracts (R_{T}˜26 min, [M−H]⁻ 579, SOM FIGS. 2 & 4). Compound 7a is the A-ring dehydration product of 5a. The amount of 7b in the derivatized plaque extracts was approaching the detection limit of the HPLC assay employed so a complete analytical investigation of this compound in all the plaque samples was not performed. The configurational assignments of compounds 7a and 7b were based on a ¹H-¹H ROESY experiment of the synthetic material 7b.

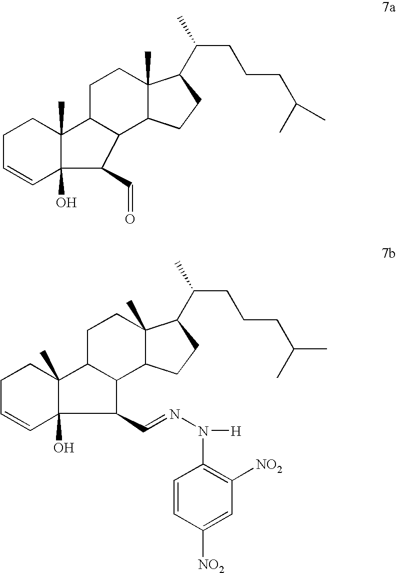

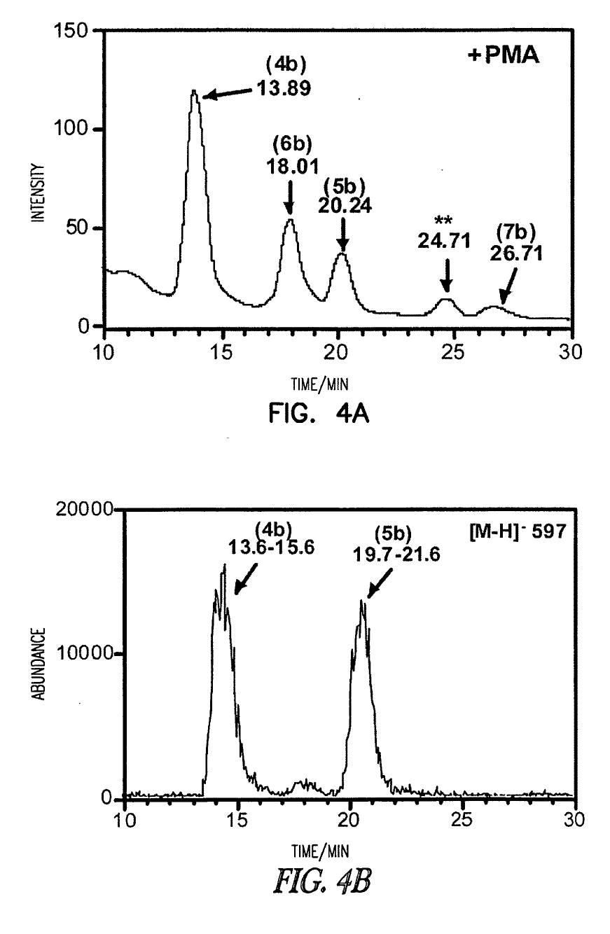

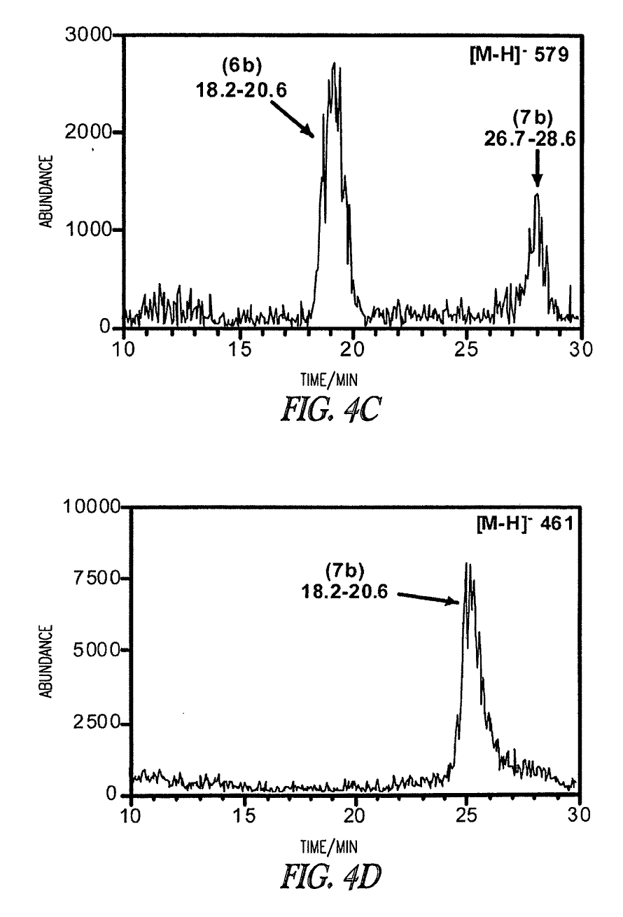

Synthesized preparations of compounds 6b, 7a, 7b, 8a and 9a were employed for identification of the compound having R_{T}˜26 min peak [M−H]⁻ 579 in FIG. 4.

HPLC-MS analysis of hydrazones. HPLC-MS analysis was performed on a Hitachi D-7000 machine, with a L-7200 autosampler (regular injection volume 10 μl), a L-7100 pump and either a L-7400 u.v. detector (360 nm) or a L-7455 diode array detector (200-400 nm) and an in-line M-8000 ion trap mass-spectrometer (in negative ion mode). The L-7100 and M-8000 were controlled using Hitachi-HSM software on a Dell GX150 PC computer. HPLC was performed using a Vydec C₁₈ reversed phase column. An isocratic mobile phase was employed (75% acetonitrile, 20% methanol and 5% water) at 0.5 mL/min. Peak height and area was determined using Hitachi D7000 chromatography station software and converted to concentrations by comparison to standard curves of authentic materials. Under these conditions the detection limit for hydrazones 4b-6b was between 1-10 nM. No resolution of the cis and trans hydrazone isomers was obtained using this HPLC system.

A representative HPLC-MS of extracted and derivatized atherosclerotic material is shown in FIG. 4. The retention times and mass ratios of several authentic samples of key hydrazone compounds are shown in Table 2.

TABLE 2
LCMS analysis of authentic hydrazones.

| hydrazone | R_{T}/min | [M − H]⁻ |
|---|---|---|
| 4b | 13.9 | 597 |
| 5b | 20.3 | 597 |
| 6b | 18.0 | 579 |
| 7b | 26.8 | 579 |
| ^{a,d}8b | 26.6 | 579 |
| ^{b}9b | 16.5 | 579 |
| ^{c}10b | 48.2 | 561 |

^{a}The hydrazone of authentic aldehyde 8a was prepared by the derivatization procedure above, the aldehyde 8a was not independently synthesized and purified.
^{b}The hydrazone of commercially-available ketone 9a was prepared by the derivatization procedure described above, and was not independently synthesized and purified.
^{c}The hydrazone of authentic aldehyde 10a was prepared by the derivatization procedure above, and was not independently synthesized and purified.
^{d}Differentiation between 8b and 9b was made based on their u.v. spectra [measured by a Hitachi L-7455 diode array detector (200-400 nm)]. The α,β-unsaturated hydrazone 8b had a λₘₐₓ of 435 nm, whereas hydrazone 9b had a λₘₐₓ of 416 nm.

Analysis of plasma samples for aldehydes 4a and 5a. Plasma samples were obtained from patients (n=8) who were scheduled to undergo carotid endarterectomy within 24 h. All such plasma samples were analyzed for the presence of 4a and 5a three days after sample collection. Control plasma samples were obtained from random patients (n=15) attending a general medical clinic and were analyzed 7 days after collection. In a typical procedure, plasma in EDTA (1 ml) was washed with dichloromethane (DCM, 3×1 mL). The combined organic fractions were evaporated in vacuo. The residue was dissolved in methanol (0.9 mL) and a solution of 2,4-dinitrophenyl hydrazine (100 μL, 0.01 M, Lancaster) and 1N HCl in ethanol was added. Nitrogen was bubbled through the solution for 5 min and then the solution was stirred for 2 h. The resultant solution was filtered through a 0.22 μm filter and the filtrate was analyzed by the HPLC assay vide supra. Preliminary investigations revealed that the amount of 5a that can be extracted from plasma decreases by about 5% per day.

### Preparation of Authentic Samples 4a, 4b, 5a, 5b, 6a, 6b, 7a, 7b, 8a, and 8b

General Methods. Unless otherwise stated, all reactions were performed under an inert atmosphere with dry reagents, solvents, and flame-dried glassware. All starting materials were purchased from Aldrich, Sigma, Fisher, or Lancaster and used as received. Ketone 9a was obtained from Aldrich. All flash column chromatography was performed using silica gel 60 (230-400 mesh). Preparative thin layer chromatography (TLC) was performed using Merck (0.25, 0.5, or 1 mm) coated silica gel Kieselgel 60 F₂₅₄ plates. ¹H NMR spectra were recorded on Bruker AMX-600 (600 MHz), AMX-500 (500 MHz), AMX-400 (400 MHz), or AC-250 (250 MHz) spectrometers. ¹³C NMR spectra were recorded on a Bruker AMX-500 (125.7 MHz) or AMX-400 (100.6 MHz) spectrometer. Chemical shifts are reported in parts per million (ppm) on the 6 scale from an internal standard. High-resolution mass spectra were recorded on a VG ZAB-VSE instrument.

3βHydroxy-5-oxo-5,6-secocholestan-6-al (4a). This compound was synthesized as generally described in K. Wang, E. Bermudez, W. A. Pryor, Steroids 58, 225 (1993). A solution of cholesterol 3 (1 g, 2.6 mmol) in chloroform-methanol (9:1) (100 ml) was ozonized at dry ice temperature for 10 min. The reaction mixture was evaporated and stirred with Zn powder (650 mg, 10 mmol) in water-acetic acid (1:9, 50 ml) for 3 h at room temperature. The reduced mixture was diluted with dichloromethane (100 ml) and washed with water (3×50 ml). The combined organic fractions were dried over sodium sulfate and evaporated to dryness in vacuo. The residue was purified using silica-gel chromatography [ethyl acetate-hexane (25:75)] to give the title compound 4a as a white solid (820 mg, 76%):

¹H NMR (CDCl₃) δ 9.533 (s, 1H, CHO), 4.388 (m, 1H, H-3), 3.000 (dd, J=14.0, 4.0 Hz, 1H, H-4e), 0.927 (s, 3H, CH₃-19), 0.827 (d, J=6.8 Hz, 3H, CH₃-21), 0.782 (d, J=6.8 Hz, 3H, CH₃), 0.778 (d, J=6.8 Hz, 3H, CH₃), 0.603 (s, 3H, CH₃-18); ¹³C NMR (CDCl₃) δ 217.90 (C-5), 202.76 (C-6), 70.81 (C-3), 55.96 (C-17), 54.26 (C-14), 52.52 (C-10), 46.70 (C-4), 44.17 (C-7), 42.43 (C-13), 42.17 (C-9), 39.75 (C-12), 39.33 (C-24), 35.85 (C-22), 35.61 (C-20), 34.58 (C-8), 33.99 (C-1), 27.87 (C-25), 27.73 (C-16), 27.52 (C-2), 25.22 (C-15), 23.62 (C-23), 22.91 (C-11), 22.70 (C-27), 22.44 (C-26), 18.44 (C-21), 17.46 (C-19), 11.42 (C-18). HRMALDITOFMS calcd for C₂₇H₄₆O₃Na (M+Na)⁺ 441.3339, found 441.3355.

2,4-Dinitrophenylhydrazone of 3β-hydroxy-5-oxo-5,6-secocholestan-6-al (4b). This compound was synthesized as generally described in K. Wang, E. Bermudez, W. A. Pryor, Steroids 58, 225 (1993). 2,4-Dinitrophenylhydrazine (52 mg, 0.26 mmol) and p-toluenesulfonic acid (1 mg, 0.0052 mmol) was added to a solution of ketoaldehyde 4a (100 mg, 0.24 mmol) in acetonitrile (10 ml). The reaction mixture was stirred for 4 h at room temperature, and evaporated to dryness in vacuo. The residue was dissolved in ethyl acetate (10 ml) and washed with water (3×20 ml). The combined organics were dried over sodium sulfate and evaporated to dryness in vacuo. The residue was purified by silica gel chromatography [ethyl acetate-hexane (1:4)] to give the title compound 4b as a yellow solid (100 mg, 70%) and as a mixture of the cis and trans isomers (1:4). Crystallization from hexane-methylene chloride gave trans-4b as yellow needles (30 mg, 21%):

¹H NMR (CDCl₃): δ 10.994 (s, 1H, NH), 9.107 (d, J=2.8 Hz, 1H, H-3′), 8.316 (dd, J=9.6, 2.8 Hz, 1H, H-5′), 7.923 (d, J=9.6 Hz, 1H, H-6′), 7.419 (dd, J=6.0, 3.6 Hz, 1H, H-6), 4.417 (m, 1H, H-3), 2.971 (dd, J=13.6, 4.0 Hz, 1H, H-4e), 1.076 (s, 3H, CH₃-19), 0.915 (d, J=6.4 Hz, 3H, CH₃-21), 0.853 (d, J=6.4 Hz, 3H, CH₃), 0.849 (d, J=6.4 Hz, 3H, CH₃), 0.710 (s, 3H, CH₃-18); ¹³C NMR (CDCl₃) δ 216.05 (C-5), 150.84 (C-6), 144.96 (C-1′), 137.87 (C-4′), 130.23 (C-5′), 128.90 (C-2′), 123.50 (C-3′), 116.52 (C-6′), 71.42 (C-3), 56.07 (C-17), 54.54 (C-14), 52.69 (C-10), 47.34 (C-4), 42.61 (C-13), 42.61 (C-9), 39.82 (C-12), 39.42 (C-24), 36.99 (C-8), 35.96 (C-22), 35.67 (C-20), 34.13 (C-1), 32.65 (C-7), 27.98 (C-16), 27.93 (C-25), 27.90 (C-2), 25.31 (C-15), 23.70 (C-23), 23.12 (C-11), 22.78 (C-27), 22.52 (C-26), 18.56 (C-21), 17.77 (C-19), 11.67 (C-18); HRMALDITOFMS calcd for C₃₃H₅₀N₄O₆Na (M+Na) 621.3622, found 621.3622: λₘₐₓ 360 nm, ε2.57±0.31×10⁴ M⁻¹cm⁻¹.

3β-Hydroxy-5β-hydroxy-B-norcholestane-6βcarboxaldehyde (5a). This compound was synthesized as generally described in T. Miyamoto, K. Kodama, Y. Aramaki, R. Higuchi, R. W. M. Van Soest, Tetrahedron Letter 42, 6349 (2001). To a solution of ketoaldehyde 4a (800 mg, 1.9 mmol) in acetonitrile-water (20:1, 100 ml) was added of L-proline (220 mg, 1.9 mmol). The reaction mixture was stirred for 2 h at room temperature, evaporated to dryness in vacuo. The residue was dissolved in ethyl acetate (50 ml) and washed with water (3×50 ml). The combined organic fractions were dried over sodium sulfate and evaporated in vacuo. The residue was purified by silica gel chromatography [ethyl acetate-hexane (1:4)] to give the title compound 5a as a white solid (580 mg, 73%):

¹H NMR (CDCl₃) δ 9.689 (d, J=2.8 Hz, 1H, CHO), 4.115 (m, 1H, H-3), 3.565 (s, 1H, 3β-OH), 2.495 (broad s, 1H, 5β-OH), 2.234 (dd, J=9.2, 3.2 Hz, 1H, H-6), 0.920 (s, 3H, CH₃-19), 0.904 (d, J=6.4 Hz, 3H, CH₃-21), 0.854 (d, J=6.8 Hz, 3H, CH₃), 0.850 (d, J=6.8 Hz, 3H, CH₃), 0.705 (s, 3H, CH₃-18); ¹³C NMR (CDCl₃) δ 204.74 (C-7), 84.26 (C-5), 67.33 (C-3), 63.89 (C-9), 56.10 (C-14), 55.67 (C-17), 50.42 (C-6), 45.47 (C-10), 44.72 (C-13), 44.22 (C-4), 40.02 (C-8), 39.67 (C-12), 39.44 (C-24), 36.15 (C-22), 35.58 (C-20), 28.30 (C-16), 27.98 (C-2), 27.91 (C-25), 26.69 (C-1), 24.55 (C-15), 23.78 (C-23), 22.78 (C-27), 22.52 (C-26), 21.54 (C-11), 18.71 (C-21), 18.43 (C-19), 12.48 (C-18). HRMALDITOFMS calcd for C₂₇H₄₆O₃Na (M+Na)⁺441.3339, found 441.3351.

2,4-Dinitrophenylhydrazone of 3β-Hydroxy-5β-hydroxy-B-norcholestane-6β-carboxaldehyde (5b). This compound was synthesized as generally described in K. Wang, E. Bermúdez, W. A. Pryor, Steroids 58, 225 (1993). 2,4-Dinitrophenylhydrazine (52 mg, 0.26 mmol) and hydrochloric acid (12 M, 2 drops) was added to a solution of aldehyde 5a (100 mg, 0.24 mmol) in acetonitrile (10 ml). The reaction mixture was stirred for 4 h at room temperature and evaporated to dryness in vacuo. The residue was dissolved in ethyl acetate (10 ml) and was washed with water (3×20 ml). The combined organic fractions were dried over sodium sulfate and evaporated to dryness in vacuo. The residue was purified by silica gel chromatography [ethyl acetate-hexane (1:4)] to give the title compound 5b as a yellow solid (90 mg, 62%) as the trans-5b phenylhydrazone:

¹H NMR (CDCl₃) 11.049 (s, 1H, NH), 9.108 (d, J=2.4 Hz, 1H, H-3′), 8.280 (dd, J=9.6, 2.6 Hz, 1H, H-5′), 7.901 (d, J=9.6 Hz, 1H, H-6′), 7.561 (d, J=7.2 Hz, 1H, H-7), 4.214 (m, 1H, H-3), 3.349 (s, 1H, 3′-OH), 2.337 (dd, J=9.2, 6.8 Hz, 1H, H-6), 0.967 (s, 3H, CH₃-19), 0.917 (d, J=6.8 Hz, 3H, CH₃-21), 0.850 (d, J=6.4 Hz, 3H, CH₃), 0.846 (d, J=6.4 Hz, 3H, CH₃), 0.713 (s, 3H, CH₃-18); ¹³C NMR (CDCl₃) δ 155.18 (C-7), 145.12 (C-1′), 137.51 (C-4′), 129.91 (C-5′), 128.64 (C-2′), 123.57 (C-3′), 116.36 (C-6′), 83.35 (C-5), 67.56 (C-3), 56.34 (C-17), 56.34 (C-9), 55.56 (C-14), 51.47 (C-6), 45.50 (C-10), 44.76 (C-13), 43.62 (C-4), 42.59 (C-8), 39.66 (C-12), 39.43 (C-24), 36.16 (C-22), 35.58 (C-20), 28.50 (C-16), 28.07 (C-2), 27.98 (C-25), 27.70 (C-1), 24.67 (C-15), 23.78 (C-23), 22.78 (C-27), 22.52 (C-26), 21.63 (C-11), 18.75 (C-21), 18.67 (C-19), 12.48 (C-18); HRMALDITOFMS calcd for C₃₃H₅₀N₄O₆Na (M+Na)+ 621.3622, found 621.3625. HPLC-MS detection: R_{T} 20.8 min; [M−H]⁻ 597; λₘₐₓ 361 nm, ε 2.47±0.68×10⁴ M⁻¹cm¹.

5-Oxo-5,6-secocholest-3-en-6-al (6a). This compound was synthesized as generally described in P. Yates, S. Stiveer, Can. J. Chem. 66, 1209 (1988). Methanesulfonyl chloride (400 μl, 2.87 mmol) was added dropwise to a stirred solution of ketoaldehyde 4a (300 mg, 0.72 mmol) and triethylamine (65 μl, 0.84 mmol) in CH₂Cl₂ (15 ml) at ice-bath temperature. The resulting solution was stirred for 30 min under argon at 0° C., triethylamine (400 μl, 2.87 mmol) was then added and the solution was warmed to room temperature. After 2 h, the reaction mixture was evaporated to dryness in vacuo. The residue was dissolved. in methylene chloride (15 ml) and washed with water (3×20 ml). The combined organic fractions were dried over anhydrous sodium sulfate and evaporated in vacuo. The crude residue was purified by silica gel chromatography [ethyl acetate-hexane (1:9)]. The fractions were evaporated to give aldehyde 6a (153 mg, 53%) as a colorless oil. ¹H NMR (CDCl₃) of shows δ 9.574 (s, 1H, CHO), 6.769 (m, 1H, H-3), 5.822 (d, J=10 Hz, 1H, H-4), 2.512 (dd, J=16.8, 3.6 Hz, 1H, H-7), 1.070 (s, 3H, CH₃-19), 0.882 (d, J=6.8 Hz, 3H, CH₃-21), 0.845 (d, J=6.8 Hz, 3H, CH₃), 0.841 (d, J=6.8 Hz, 3H, CH₃), 0.674 (s, 3H, CH₃-18); ¹³C NMR (CDCl₃) δ 208.22 (C-5), 202.42 (C-6), 147.46 (C-3), 128.44 (C-4), 56.08 (C-17), 54.96 (C-14), 47.80 (C-10), 45.05 (C-7), 42.33 (C-13), 42.04 (C-9), 39.73 (C-12), 39.43 (C-24), 35.93 (C-22), 35.71 (C-20), 35.42 (C-1), 33.77 (C-8), 27.97 (C-25), 27.67 (C-16), 25.22 (C-15), 24.67 (C-2), 23.71 (C-23), 23.27 (C-11), 22.77 (C-27), 22.51 (C-26), 18.54 (C-21), 17.71 (C-19), 11.48 (C-18). HRMALDITOFMS calcd for C₂₇H₄₅O₂ (M+H)⁺ 401.3414, found 401.3404.

2,4-Dinitrophenylhydrazone of 5-oxo-5,6-secocholest-3-en-6-al (6b) 2,4-Dinitrophenylhydrazine (45 mg, 0.23 mmol) was added to a solution of ketoaldehyde 6a (80 mg, 0.2 mmol) and p-toluenesulfonic acid (1 mg, 0.0052 mmol) in acetonitrile (10 ml). The reaction mixture was stirred for 2 h at room temperature and evaporated to dryness in vacuo. The residue was dissolved in methylene chloride (10 ml) and was washed with water (3×20 ml). The combined organic fractions were dried over sodium sulfate and evaporated to dryness in vacuo. The residue was purified by silica gel chromatography [ethyl acetate-hexane (15:85)] to give the title compound 6b as a yellow solid (70 mg, 60%):

trans-6b ¹H NMR (CDCl₃) shows δ 10.958 (s, 1H, NH), 9.104 (d, J=2.4 Hz, 1H, H-3′), 8.288 (dd, J=9.8, 2.8 Hz, 1H, H-5′), 7.896 (d, J=9.6 Hz, 1H, H-6′), 7.337 (dd, J=5.6, 5.6 Hz, 1H, H-6), 6.771 (m, 1H, H-3), 5.822 (d, J=10 Hz, 1-H, H-4), 2.600 (ddd, J=16.4, 4.8, 4.8 Hz, 1H, H-7), 1.139 (s, 3H, CH₃-19), 0.897 (d, J=6.4 Hz, 3H, CH₃-21), 0.840 (d, J=6.8 Hz, 3H, CH₃), 0.837 (d, J=6.8 Hz, 3H, CH₃), 0.703 (s, 3H, CH₃-18); ¹³C NMR (CDCl₃) δ 207.78 (C-5), 151.17 (C-6), 147.69 (C-3), 145.00 (C-1′), 137.61 (C-4′), 129.97 (C-5′), 128.52 (C-2′), 128.38 (C-4), 123.48 (C-3′), 116.46 (C-6′), 56.05 (C-17), 54.68 (C-14), 47.87 (C-10), 42.30 (C-13), 41.69 (C-9), 39.72 (C-12), 39.37 (C-24), 36.35 (C-8), 35.91 (C-22), 35.66 (C-20), 35.34 (C-1), 32.84 (C-7), 27.93 (C-25), 27.73 (C-16), 24.93 (C-15), 24.68 (C-2), 23.69 (C-23), 23.24 (C-11), 22.74 (C-27), 22.48 (C-26), 18.52 (C-21), 17.81 (C-19), 11.58 (C-18); HRMALDITOFMS calcd for C₃₃H₄₈N₄O₅Na (M+Na)⁺ 603.3517, found 603.3523. HPLC-MS detection: R_{T} 18.3 min; [M−H]⁻ 579; λₘₐₓ 360 nm, ε 2.29±0.23×10⁴ M¹cm⁻¹.

5β-Hydroxy-B-norcholest-3-ene-6β-carboxaldehyde (7a). This compound was synthesized as generally described in P. Yates, S. Stiveer, Can. J. Chem. 66, 1209 (1988). Sodium methoxide in methanol (0.5 M, 0.16 mmol) was added dropwise to a solution of ketoaldehyde 4a (50 mg, 0.125 mmol) in anhydrous methanol (10 ml) under an argon atmosphere at room temperature. After 30 min, the methanol was removed in vacuo, and the residue was dissolved in dichloromethane (20 ml) washed with water (3×20 ml). The combined organic fractions were dried over sodium sulfate, and evaporated in vacuo. The residue was purified by silica gel chromatography [ethyl acetate-hexane (1:9)] to give the title aldehyde 7a as a colorless oil (16 mg, 32%):

¹H NMR (CDCl₃) δ 9.703 (d, J=3.2, 1H, CHO), 5.716 (m, 2H, H-3 and H-4), 2.398 (dd, J=9.6, 3.6 Hz, 1H, H-6), 0.953 (s, 3H, CH₃-19), 0.904 (d, J=6.4 Hz, 3H, CH₃-21), 0.854 (d, J=6.4 Hz, 3H, CH₃), 0.849 (d, J=6.4 Hz, 3H, CH₃), 0.706 (s, 3H, CH₃-18); ¹³C NMR (CDCl₃) δ204.41 (C-7), 134.21 (C-3), 126.66 (C-4), 81.44 (C-5), 64.49 (C-9), 55.86 (C-14), 55.55 (C-17), 48.44 (C-6), 45.12 (C-10), 44.47 (C-13), 39.92 (C-8), 39.45 (C-12), 39.40 (C-24), 36.16 (C-22), 35.57 (C-20), 29.06 (C-1), 28.31 (C-16), 27.98 (C-25), 24.73 (C-15), 23.76 (C-23), 22.78 (C-27), 22.53 (C-26), 21.69 (C-2), 21.24 (C-11), 18.74 (C-21), 18.44 (C-19), 12.37 (C-18); HRMALDITOFMS calcd for C₂₇H₄₄O₂Na (M+Na)⁺ 423.3233, found 423.3240.

2,4-Dinitrophenylhydrazone of 5β-hydroxy-B-norcholest-3-ene-6β-carboxaldehyde (7b): 2,4-Dinitrophenylhydrazine (8 mg, 0.041 mmol) and p-toluenesulfonic acid (1 mg, 5.2 μmol) were added to a solution of aldehyde 7a (15 mg, 0.037 mmol) in acetonitrile (5 ml). The reaction mixture was stirred 2 h at room temperature, evaporated under vacuum and diluted with methylene chloride (10 ml). The organic layer was washed with water (3×20 ml), dried over sodium sulfate and evaporated to dryness. The residue purified by silica gel chromatography [ethyl acetate-hexane (1:9)] to give hydrazone 7b as a yellow solid (9 mg, 41%):

¹H NMR (CDCl₃) trans-7b 11.060 (s, 1H, NH), 9.119 (d, J=2.8 Hz, 1H, H-3′), 8.291 (dd, J=9.2, 2.0 Hz, 1H, H-5′), 7.930 (d, J=9.6 Hz, 1H, H-6′), 7.546 (d, J=7.2 Hz, 1H, H-7), 5.761 (ddd, J=10.2, 4.4, 2.0 Hz, 1H, H-3), 5.705 (d, J=9.6 Hz, 1H, H-4), 2.485 (dd, J=10.4, 7.6 Hz, 1H, H-6), 0.977 (s, 3H, CH₃-19), 0.917 (d, J=6.4 Hz, 3H, CH₃-21), 0.848 (d, J=6.8 Hz, 3H, CH₃), 0.844 (d, J=6.4 Hz, 3H, CH₃), 0.707 (s, 3H, CH₃-18); ¹H-¹H ROESY NMR significant correlations (H₄-H₆), (H₆-H₇), (H₇-H₈), (H₇-H₁₉), missing correlations (H₃-H₁₉), (H₄-H₇), (H₄—H₁₉), (H₆-H₁₉); ¹³C NMR (CDCl₃) δ 154.62 (C-7), 145.09 (C-1′), 137.59 (C-4′), 133.89 (C-3), 129.94 (C-5′), 128.68 (C-2′), 127.12 (C-4), 123.57 (C-3′), 116.42 (C-6′), 80.91 (C-5), 56.83 (C-9), 56.07 (C-14), 55.39 (C-17), 49.58 (C-6), 45.00 (C-10), 44.58 (C-13), 42.50 (C-8), 39.44 (C-12), 39.44 (C-24), 36.17 (C-22), 35.54 (C-20), 30.46 (C-1), 28.53 (C-16), 27.98 (C-25), 24.91 (C-15), 23.74 (C-23), 22.77 (C-27), 22.52 (C-26), 21.79 (C-2), 21.31 (C-1), 18.76 (C-21), 18.76 (C-19), 12.34 (C-18). HPLC-MS detection: R_{T} 18.3 min; [M−H]⁻ 579; λₘₐₓ 364 nm, ε 2.32±0.17×10⁴ M⁻¹cm⁻¹.

3β-Hydroxy-B-norcholest-5-ene-6-carboxaldehyde (8a) A solution of aldehyde 5a (50 mg, 0.12 mmol) and phosphoric acid (85%, 5 ml) in acetonitrile-methylene chloride (1:1, 4 ml) was heated under reflux for 30 min. The reaction mixture was evaporated in vacuo, diluted with methylene chloride (50 ml), washed with water (3×20 ml). The organic layer was dried over sodium sulfate and evaporated under vacuum. The residue was purified by liquid chromatography on silica gel with ethyl acetate-hexane (1:4) to give the title aldehyde 12 mg (25%) of α,β-unsaturated aldehyde 8a: The ¹H NMR (CDCl₃) of 8a shows δ 9.958 (s, 1H, CHO), 3.711 (tt, J=10.8, 4.5 Hz, 1H, H-3), 3.475 (dd, J=14.1, 4.8, 1H, H-4), 2.563 (dd, J=11.0, 11.0 Hz, 1H, H-8), 0.953 (s, 3H, CH₃-19), 0.941 (d, J=6.9 Hz, 3H, CH₃-21), 0.881 (d, J=6.6 Hz, 3H, CH₃), 0.876 (d, J=6.6 Hz, 3H, CH₃), 0.746 (s, 3H, CH₃-18); ¹³C NMR (CDCl₃) δ 189.44 (C-7), 168.74 (C-5), 139.21 (C-6), 70.88 (C-3), 60.16 (C-9), 55.40 (C-17), 54.48 (C-14), 46.35 (C-10), 46.19 (C-8), 45.27 (C-13), 39.86 (C-12), 39.55 (C-24), 36.26 (C-4), 36.22 (C-22), 35.64 (C-20), 33.93 (C-1), 31.32 (C-2), 28.62 (C-16), 28.09 (C-25), 26.65 (C-15), 24.00 (C-23), 22.90 (C-27), 22.64 (C-26), 20.80 (C-11), 19.02 (C-21), 15.73 (C-19), 12.59 (C-18); HRMS calcd for C₂₇H₄₄O₂Na (M+Na)⁺ 423.3233, found 423.3239.

B-norcholest-3,5-diene-6-carboxaldehyde 12a a white solid (27 mg, 60%), was obtained as a side-product from this reaction: The ¹H NMR (CDCl₃) δ 10.017 (s, 1H, CHO), 6.919 (d, J=10.2 Hz, 1H, H-4), 6.225 (m, 1H, H-3), 2.675 (dd, J=10.8, 10.8 Hz, 1H, H-8), 0.950 (d, J=6.9 Hz, 3H, CH₃-21), 0.914 (s, 3H, CH₃-19), 0.882 (d, J=6.8 Hz, 3H, CH₃), 0.877 (d, J=6.8 Hz, 3H, CH₃), 0.769 (s, 3H, CH₃-18); ¹³C NMR (CDCl₃) δ 189.41 (C-7), 163.33 (C-5), 138.18 (C-6), 135.75 (C-3), 120.68 (C-4), 59.54 (C-9), 55.41 (C-17), 54.30 (C-14), 45.47 (C-8), 45.08 (C-10), 44.72 (C-13), 39.79 (C-12), 39.55 (C-24), 36.27 (C-22), 35.65 (C-20), 34.18 (C-1), 28.62 (C-16), 28.09 (C-25), 26.72 (C-15), 24.00 (C-23), 23.96 (C-2), 22.90 (C-27), 22.64 (C-26), 20.72 (C-11), 19.03 (C-21), 14.87 (C-19), 12.62 (C-18); HRMALDITOFMS calcd for C₂₇H₄₃O (M+H)⁺ 383.3308, found 383.3309.

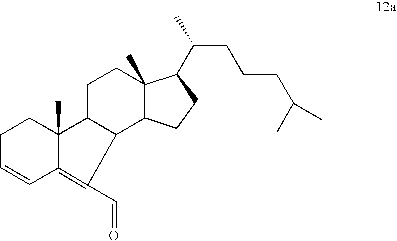

Aldolization of ketoaldehyde 4a with amino acids. In a typical procedure, ketoaldehyde 4a (2 mg, 4.8 μmol) was dissolved in DMSO-d₆ (800 μl) and D₂O (80 μl) in an NMR tube. To this solution was added 1 equivalent of either: a) L-proline, b) glycine, c) L-lysine hydrochloride or d) L-lysine ethyl ester dihydrochloride. At time points the samples were analyzed by ¹H NMR. The reaction was followed routinely by monitoring changes in a number of resonances in the ¹H NMR (DMSO-d₆) ¹H NMR 5a shows δ 9.527 (d, J=3.2 Hz, 1H, CHO), 3.876 (m, 1H, H-3), 0.860 (d, J=6.4 Hz, 3H, CH₃-21), 0.772 (d, J=6.8 Hz, 3H, CH₃), 0.767 (d, J=6.8 Hz, 3H, CH₃), 0.771 (s, 3H, CH₃-19), 0.642 (s, 3H, CH₃-18). ¹H NMR 4a shows δ 9.518 (s, 1H, CHO), 4.223 (m, 1H, H-3), 2.994 (dd, J=12.8, 4.0 Hz, 1H, H-4e), 0.858 (d, J=6.8 Hz, 3H, CH₃), 0.842 (s, 3H, CH₃-19), 0.811 (d, J=6.8 Hz, 3H, CH₃), 0.807 (d, J=6.4 Hz, 3H, CH₃-21), 0.615 (s, 3H, CH₃-18). Under these conditions, no aldolization of 4a occurs in DMSO-d₆ (800 μl) and D₂O (80 μl).

Aldolization of secoketoaldehyde 4a with atherosclerotic artery and bloodfractions. In a typical procedure, ketoaldehyde 4a (5 mg, 0.0012 mmol) was dissolved in DMSO-d₆ (800 μl) and D₂O (80 μl). To this solution was added either a) atherosclerotic artery (2.1 mg) that had been homogenized in PBS (1 ml) in a tissue homogenizer and then lyophilized to dryness, b) lyophilized human blood (1 ml), c) lyophilized human plasma (1 ml) or d) PBS lyophilized (1 ml). At time points samples were removed and analyzed by ¹H NMR vide supra. Under these conditions no aldolization of 4a occurred in the presence of lyophilized PBS.

Biological Investigations with 4a and 5a

Some oxysterols have been described that are generated by oxidation of cholesterol in vivo. E. Lund, 1. Björkhem, Acc. Chem. Res. 28, 241 (1995). Moreover, an analogue of 5a that differs structurally only in the cholestan side chain has been isolated from the marine sponge Stelletta hiwasaensis as part of a general screen for cytotoxic natural products. T. Miyamoto, K. Kodama, Y. Aramaki, R. Higuchi, R. W. M. Van Soest, Tetrahedron Lett. 42, 6349 (2001); B. Liu, Z. Weishan, Tetrahedron Lett. 43, 4187 (2002). However, derivatives where the steroid nucleus is disrupted, as in sterols 4a and 5a, have not previously been reported in humans.

Cytotoxicity assays. WI-L2 human B-lymphocyte line, HAAE-1 human abdominal aortic endothelial line, MH-S murine alveolar macrophage line, and J774A.1 murine tissue macrophage line were obtained from the ATCC. Human aortic endothelial cells (HAEC) and human vascular smooth muscle cells (VSMS) were obtained from Cambrex Bio Science. Jurkat E6-1T-lymphocytes were kindly provided by Dr. J. Kaye (The Scripps Research Institute). Cells were cultured in ATCC-recommended media with 10% fetal calf serum. Cells were incubated in a controlled atmosphere at 37° C., with 5 or 7% CO₂. For lactate dehydrogenase (LDH) release assays, adherent cells were harvested either by addition of 0.05% trypsin/EDTA or by scraping. The cells obtained were seeded onto 96-well microtiter plates (25,000 cells/well) and allowed to recover for 24-48 h. Cells were washed gently and media replaced with fresh media containing 5% fetal calf serum. Duplicate or greater numbers of cell samples were treated with either 3, 4a or 5a (0-100 μM) for 18 h. Cytotoxicity was then determined by measuring lactate dehydrogenase (LDH) release from cells in culture. Briefly, LDH activity was measured in the cell supernatant using the CytoTox 96 Non-Radioactive Cytotoxicity Assay (Promega, USA) of cells cultured in 96-well plates at the end of the treatment period with either ketoaldehyde 4a, aldol 5a, or cholesterol 3. 100% Cytotoxicity was defined as the maximum amount of LDH released by dead cells as shown by trypan blue exclusion, or the highest amount of LDH detected upon lysis of cells by 0.9% Triton X-100. The IC₅₀ values were determined by comparison of the raw duplicate data for concentration versus cytotoxicity (%) to non-linear regression analysis (Hill plot) using Graphpad v3.0 software for Macintosh.

Lipid-loading assay (foam cell formation). J774.1 macrophages were incubated in ATCC-recommended media containing 10% fetal bovine serum under a controlled atmosphere of 5 or 7% CO₂ at 37° C., in 8-well chamber slides. Cells were then incubated for 72 h in the same media containing the antioxidants 2,6-di-tert-butyl-4-methylphenol toluene (100 μM), diethylenetriamine-pentaacetic acid (100 μM) and either LDL (100 μg/mL), LDL (100 μg/mL) and atheronal-A 4a (20 μM) or LDL (100 μg/mL) and atheronal-B 5a (20 μM). At termination, cells were washed twice with PBS (pH 7.4). The cells were then fixed with 6% (v/v) paraformaldehyde in PBS for 30 minutes, rinsed with propylene glycol for 2 minutes and lipids were stained with 5 mg/ml Oil Red 0 for 8 minutes. The cells were counterstained with Harris' hematoxylin for 45 seconds, and background staining was removed with 6% paraformaldehyde followed by washing once in PBS and once in tap water. Cover slips were mounted onto the glass slides using glycerol and the slide preparations were examined by light microscopy. The number of lipid-laden cells was scored out of a total of at least 100 cells counted in a single field in each slide, and expressed as a percentage of total cells. Photographs were taken at 100× magnification.

Circular dichroism Circular dichroism (CD) spectra of LDL (100 μg/ml), LDL (100 μg/ml) and 4a (10 μM), and LDL (100 μg/ml) and 5a (10 μM) in PBS (pH 7.4 with 1% isopropanol) were recorded at 37° C. on an Aviv spectropolarimeter, in thermostatically controlled (+0.1° C.) 0.1 cm quartz cuvettes. Spectra were recorded in the peptidic range (200-260 nm). To increase the signal-to-noise ratio, multiple spectra (three) were averaged for each measurement. The deconvolution of the molar elipticity spectra for each measurement was performed using the CDPro suite of software (by Narasimha Sreerama from Colorado State University) on a Dell PC.

### Example 2

### Athersosclerotic Plaques Generate Ozone and Cholesterol Ozonolysis Products

Using the methods described hereinabove, this Example shows that atherosclerotic tissue, obtained by carotid endarterectomy from 15 human patients (n=15), can produce ozone detectable by reaction with indigo carmine 1.

### Bleaching of Indigo Carmine by Ozone Produced by Atherosclerotic Plaques

The inventors have previously that when antibody-coated white cells were treated with the protein kinase C activator, 4-β-phorbol 12-myristate 13-acetate (PMA), in a solution of indigo carmine 1 (a chemical trap for ozone), the visible absorbance of indigo carmine 1 was bleached and indigo carmine 1 was converted into isatin sulfonic acid 2. See, e.g., P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003). The structure of isatin sulfonic acid 2 is provided in FIG. 1A. When these experiments were performed in H₂¹⁸O (>95% ¹⁸O), isotope incorporation into the lactam carbonyl of isatin sulfonic acid 2 was observed. Id. This procedure distinguished ozone and ¹O₂* from other oxidants that may also oxidize indigo carmine 1, because among the oxidants thought to be associated with inflammation, only ozone oxidatively cleaves the double bond of indigo carmine 1 with isotope incorporation (from in H₂¹⁸O) into the lactam carbonyl of isatin sulfonic acid 2 (see id. and FIG. 1A).

As described in Example 1, plaque material was obtained by carotid endarterectomy from 15 human patients believed to have problematic atherosclerosis. Each plaque was split into two equal portions (about 50 mg wet weight suspended in 1 mL of PBS). Each portion of plaque material was added to a solution of indigo carmine 1 (200 μM) and bovine catalase (50 μg/mL) in phosphate buffered saline (PBS, pH 7.4, 10 mM phosphate buffer, 150 mM NaCl) (1 mL). The analysis was initiated by addition of DMSO (10 μL) or phorbal myristate (PMA, 10 μL, 20 μg/mL) in DMSO to one or the other aliquot of suspended plaque materials.

Bleaching of the visible absorbance of 1 was observed in 14 of the 15 plaque samples upon PMA addition (FIG. 1B). This bleaching was accompanied by formation of isatin sulfonic acid 2 as determined by reversed-phase HPLC analysis (FIGS. 1A and C). The amount of isatin sulfonic acid 2 formed varied from 1.0 to 262.1 nmol/mg depending upon the plaque isolate tested. The mean amount of isatin sulfonic acid 2 generated by the different isolates was 72.62±21.69 nmol/mg.

When the PMA activation of suspended plaque material was performed in H₂¹⁸O-containing PBS (>95% ¹⁸O) (n=2) with indigo carmine 1 (200 μM), approximately 40% of the lactam carbonyl oxygen of indigo carmine 1 incorporated ¹⁸O, as shown by the relative intensities of the [M−H]⁻ 228 and 230 mass fragment peaks in the mass spectrum of the isolated cleaved product isatin sulfonic acid 2 (FIG. 1D).

These studies with indigo carmine 1 indicate that ozone was produced by activated atherosclerotic plaque material.

### Ozonolysis Products of Cholesterol

One of the major lipids present in atherosclerotic plaques is cholesterol 3. D. M. Small, Arteriosclerosis 8, 103 (1988). In a chemical model study, workers have shown that amongst a panel of oxidants such as, ³O₂, ¹O₂*, .O₂⁻, O₂²⁻, hydroxyl radical, O₃ and .O₂⁺ and ozone O₃, only ozone cleaves the Δ^{5,6} double bond of cholesterol 3 to yield the 5,6-secosterol 4a (FIG. 2A). This observation is in agreement with other chemical reports, which also indicate that the 5,6-secosterol 4a is the principle product of cholesterol 3 ozonolysis. Gumulka et al. J. Am. Chem. Soc. 105, 1972 (1983); Jaworski et al., J. Org. Chem. 53, 545 (1988); Paryzek et al., J. Chem. Soc. Perkin Trans. 1, 1222 (1990); Cornforth et al., Biochem. J. 54, 590 (1953).

Further experiments were therefore directed toward detecting and identifying whether the 5,6-secosterol 4a or other ozonolysis products of cholesterol were present in atherosclerotic plaques. Human atherosclerotic plaques of 14 patients (n=14) were therefore searched for the presence of the 5,6-secosterol 4a both prior to and after activation with PMA.

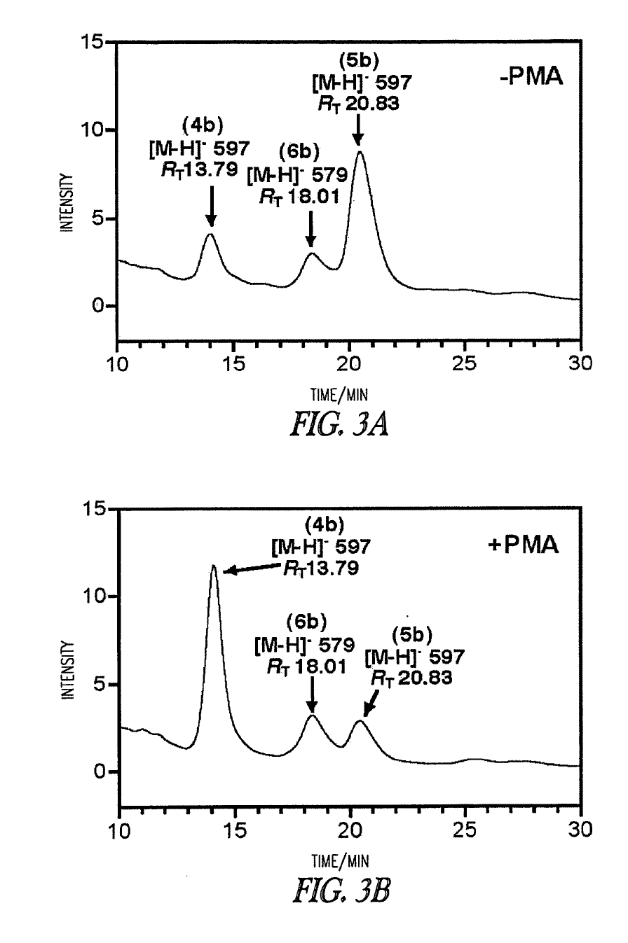

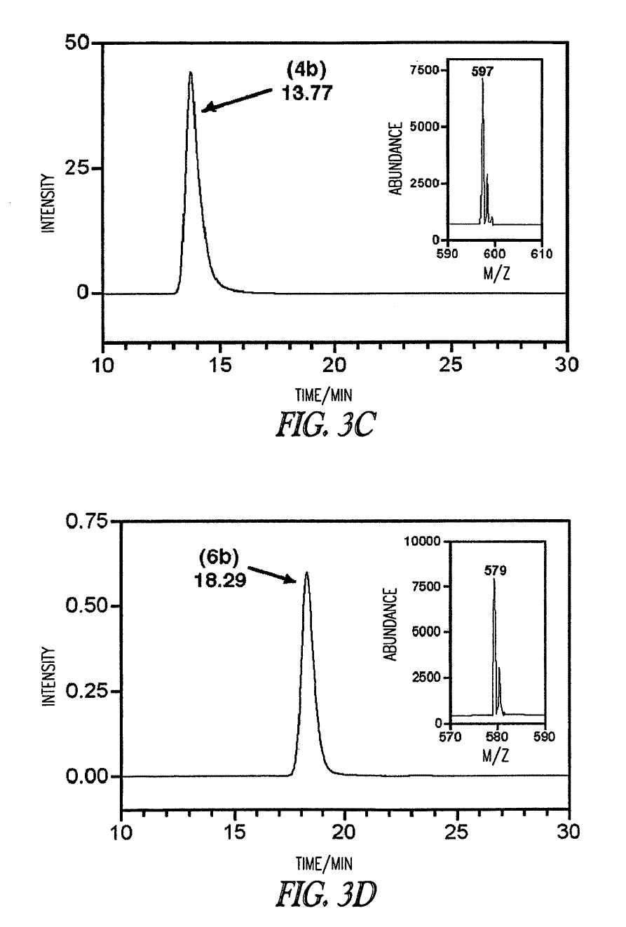

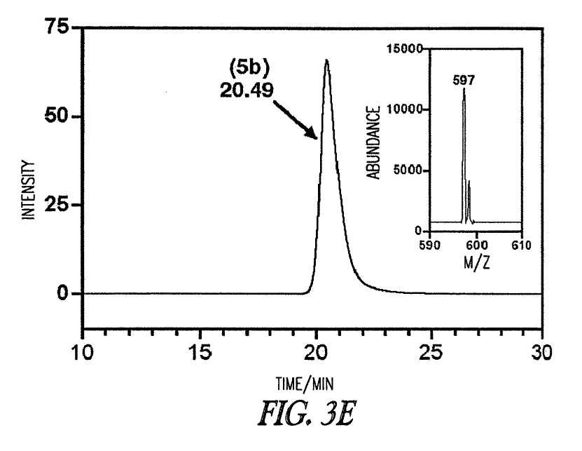

A modification of the analytical procedure developed by Pryor and colleagues was used for these studies. See K. Wang, E. Bermúdez, W. A. Pryor, Steroids 58, 225 (1993). This modified process involved extraction of a suspension of the homogenized plaque material (about 50 mg wet weight) in PBS (1 mL, pH 7), with an organic solvent (methylene chloride, 3×5 mL) followed by treatment of the organic fraction with an ethanolic solution of 2,4-dinitrophenylhydrazine hydrochloride (DNPH HCl) (2 mM in ethanol at pH 6.5) for 2 h at room temperature. This reaction mixture was analyzed by HPLC (direct injection, u.v. detection at 360 nm) and in-line negative ion electrospray mass-spectroscopy for the presence of 4b, the 2,4-dinitrophenylhydrazone derivative of the ozonolysis product 4a (FIG. 3). The hydrazone 4b was detected in 11 of the 14 unactivated plaques extracts (between 6.8 and 61.3 pmol/mg of plaque) and in all activated plaque extracts (between 1.4 and 200.6 pmol/mg). Furthermore, the amount of 4a, as judged by the mean amount of 4b, in the plaque materials significantly increased upon activation with PMA. In particular, when no PMA was used, the mean amount of 4b was 18.7±5.7 pmol/mg. In contrast, when PMA was added, the mean amount of 4b was 42.5±13.6 pmol/mg (n=14, p<0.05) (FIG. 3A-B).

In addition to 4b, two other major hydrazone peaks were observed during HPLC analysis of plaque extracts. The first peak had a R_{T}˜20.5 min and [M−H]⁻=597 and the second had a R_{T}˜18.0 min and [M−H]⁻ 579 (FIGS. 3A,B). The hydrazone 4b was readily distinguishable from these peaks because it had a retention time of about 13.8 min (R_{T}˜13.8 min, [M−H]⁻ 597) (FIGS. 3A,B). By comparison with authentic samples, the peak with a R_{T}˜20.8 min was determined to be the hydrazone derivative 5b of the aldol condensation product 5a (FIGS. 2 and 3E). In chemical model studies, Pryor had previously noted that a major side-product of the hydrazine derivatization of 4a was the hydrazone derivative 5b of the aldol condensation product 5a, and the relative amount of which was a function of both acid concentration and reaction time. K. Wang, E. Bermudez, W. A. Pryor, Steroids 58, 225 (1993).

The extent of conversion of 4a into 5b under the conditions of derivatization employed was about 20%, over the range of 4a concentrations tested (5 to 100 μM). However, more than 20% conversion was often observed. The measured amount of 5a that exceeded 20% of the 4a present in the same plaque sample likely arose from ozonolysis of 3 followed by aldolization.

Many biochemical constituents that contain amino or carboxylate groups may catalyze aldolization reactions. Such components are present in plaques and blood, and may facilitate the conversion of 4a into 5a. Further experimentation indicated that the following amino acids and materials facilitated conversion of 4a into 5a: L-Pro (2 h, complete conversion), Gly (24 h, complete conversion), L-Lys.HCl (24 h, complete conversion), L-Lys(OEt).2HCl (100 h, 62% conversion) as well as extracts from atheromatous arteries (22 h, complete conversion), whole blood (15 h, complete conversion), plasma (15 h, complete conversion) and serum (15 h, complete conversion). All such agents accelerated the conversion of 4a into 5a relative to the rate of the background reaction.

As described above, the amount of ketoaldehyde 4a within the plaques increased upon PMA activation. However, the effect of PMA on formation of 5a was less clear. In some cases, the levels of 5a increased after PMA activation (FIG. 5B, patients F and H) while in other cases the levels of 5a decreased after PMA activation (FIG. 5B, patients C, G and N).

A number of carbonyl-containing steroid-derivatives 6a-9a whose 2,4-dinitrophenylhydrazone derivatives had a peak [M−H]⁻ of 579 in the mass spectrum (FIG. 2B) were synthesized and analyzed to assist in the identification of the peak at 18 min [M−H]⁻ 579 (FIGS. 3A,B). By comparison to HPLC coinjection, negative electrospray mass-spectrometry and u.v. spectra of authentic samples, the peak at ˜18 min was determined to be 6b, the hydrazone derivative of 6a, and the A-ring dehydration product of 4a (FIG. 3D). The extent of conversion of 4a into 6b was investigated under the standard conditions selected for derivatization. This extent of conversion was consistently found to be less than 2% over the range of 4a concentrations tested (5 to 100 μM). These data indicate that the amount of 6a present within a plaque extract that exceeded 2% of the amount of ketoaldehyde 4a within that extract, was present prior to derivatization and arose from ozonolysis product 4a by β-elimination of water.

In addition to the three major hydrazone products 4b-6b, another product 7b, was detected and determined to be the hydrazone derivative of 7a, and the A-ring dehydration product of 5a. This product (7b) was present in trace amounts (<5 pmol/mg) in several plaque extracts and had a retention time of about 26 min ([M−H]⁻ 579, FIG. 4). However, the amount of 7b in the plaque extracts was approaching the detection limit of the HPLC assay employed, and a complete investigation as to the presence or absence of this compound in all the plaque samples has not yet been performed.

The experimental evidence that activated plaque material oxidatively cleaves the double bond of indigo carmine 1 with the chemical signature of ozone and that the Δ^{5,6}-double bond of cholesterol is cleaved by a pathway that, according to known chemistry, is unique to ozone gives compelling evidence that atherosclerotic plaques can generate ozone. Furthermore, since these unique ozone oxidation products of cholesterol are also present prior to plaque activation it is likely that ozone is also generated during the evolution of the atherosclerotic plaque.

It is well established that exogenously administered ozone is pro-inflammatory in vivo, via activation of interleukin (IL)-1α, IL-8, interferon (IFN)-γ, platelet aggregating factor (PAF), growth-related oncogene (Gro)-α, nuclear factor (NF)-κB and tumor necrosis factor (TNF)-α. In addition to these generally known effects of ozone in inflammation, there are circumstances unique to the atherosclerotic plaque that may increase the pathological role of endogenously-generated ozone for the initiation and perpetuation of disease when it is produced at this site. The ozonolysis of cholesterol may be unique to the plaque because it is only at this site where the requisite high concentration of ozone and cholesterol occur in the absence of other reactive substances that could trap any generated ozone.

In so far as atherosclerotic arteries contain both antibodies and a ¹O₂* generating system, in the form of activated macrophages and myeloperoxidase, it is likely that atherosclerotic lesions can generate O₃ via the antibody-catalyzed water oxidation pathway. Indeed, the observation that the Δ^{5,6}-double bond of 3 is cleaved to give 4a is further evidence for the production of ozone by antibody catalysis in inflammation. Many oxysterols are known to be generated by oxidation of cholesterol in vivo and an analogue of 5a that differs structurally only in the cholestan side chain has been isolated from the marine sponge Stelletta hiwasaensis as part of a general screen for cytotoxic natural products. T. Miyamoto, K. Kodama, Y. Aramaki, R. Higuchi, R. W. M. Van Soest, Tetrahedron Letter 42, 6349 (2001); B. Liu, Z. Weishan, Tetrahedron Lett. 43, 4187 (2002). However, derivatives where the steroid nucleus has been disrupted, as in sterols 4a-6a, have to our knowledge never before been reported in man. Therefore it is important to instigate a search for other such steroids and their derivatives and investigate their biological functions.

### Example 3

### Cholesterol Ozonolysis Products Exist in the Bloodstream of Atherosclerosis Patients

The inventors have previously shown that ozone is generated during the antibody-catalyzed water oxidation pathway and that ozone, as a powerful oxidant, could play a role in inflammation. P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003).

Inflammation is thought to be a factor in the pathogenesis of atherosclerosis. R. Ross, New Engl. J. Med 340, 115 (1999); G. K. Hansson, P. Libby, U. Schönbeck, Z.-Q. Yan, Circ. Res. 91, 281 (2002). However, prior to the invention, no specific non-invasive method has been available that could distinguish inflammatory artery disease from other inflammatory processes. The unique composition of the atherosclerotic plaque, and the products released by atherosclerotic plaque materials into the bloodstream, may provide such a method. In particular, atherosclerotic lesions contain a high concentration of cholesterol. As shown herein, ozone is generated by atherosclerotic lesions and cholesterol ozonolysis products such as 4a and/or its aldolization product 5a are also generated by atherosclerotic lesions. Hence, further experiments were performed to ascertain whether such cholesterol ozonolysis products could be a marker for inflammatory artery diseases such as atherosclerosis.

Plasma samples from two cohorts of patients were analyzed for the presence of either 4a or 5a. Cohort A was comprised of patients (n=8) that had atherosclerosis disease states that were sufficiently advanced to warrant endarterectomy. Cohort B patients were randomly selected patients that had attended a general medical clinic. In six of eight patients in cohort A, aldol 5a was detected, in amounts ranging from 70-1690 nM (˜1-10 nM is the detection limit of the assay) (FIG. 5A-C). In only one of the fifteen plasma samples from cohort B was there detectable 5a. No ketoaldehyde 4a was detected in any patient's blood sample (˜1-10 nM is the detection limit of the assay). These data indicate that either 4a is converted into 5a by catalysts contained in the blood, or that components within the plasma have differential affinity for 4a and 5a.

In the past, serum analysis of “oxysterols” has been fraught with difficulty due to problems of cholesterol auto-oxidation. H. Hietter, P. Bischoff, J. P. Beck, G. Ourisson, B. Luu, Cancer Biochem. Biophys. 9, 75 (1986). However, as described herein, amongst all the oxidation products of cholesterol generated by biologically relevant oxidation of cholesterol 3, steroid derivatives 4a and 5a are unique to ozone. These studies indicate that the presence of the aldolization product 5a in plasma, detected as its DNP hydrazone derivative 5b, can be a marker for advanced arterial inflammation in atherosclerosis. Hence, the antibody-catalyzed generation of ozone may link the otherwise seemingly independent factors of cholesterol accumulation, inflammation, oxidation and cellular damage into the pathological cascade that leads to atherosclerosis Some studies indicate that cholesterol oxidation products possess biological activities such as cytotoxicity, atherogenicity and mutagenicity. H. Hietter, P. Bischoff, J. P. Beck, G. Ourisson, B. Luu, Cancer Biochem. Biophys. 9, 75 (1986); J. L. Lorenso, M. Allorio, F. Bernini, A. Corsini, R. Fumagalli, FEBS Lett. 218, 77 (1987); A. Sevanian, A. R. Peterson, Proc. Natl. Acad. Sci. U.S.A. 81, 4198 (1984). Given that the cholesterol oxidation products 4a and 5a have never before been considered to occur in man, the effect of these compounds on key aspects of atherogenesis were further investigated as described below.

### Example 4

### Cytotoxicity of Cholesterol Ozonolysis Products

Some cholesterol oxidation products possess biological activities such as cytotoxicity, atherogenicity and mutagenicity. In this Example, the cytotoxic effects of 4a and 5a against a variety of cell lines were analyzed.

The following cell lines were employed in this study: a human B-lymphocyte (WI-L2) described in Levy et al., Cancer 22, 517 (1968); a T-lymphocyte cell line (Jurkat E6.1) described in Weiss et al., J. Immunol. 133, 123 (1984); a vascular smooth muscle cell line (VSMC) and an abdominal aorta endothelial (HAEC) cell line described in Folkman et al., Proc. Natl. Acad. Sci. U.S.A. 76, 5217 (1979); a murine tissue macrophage (J774A.1) described in Ralph et al., J. Exp. Med. 143, 1528 (1976); and an alveolar macrophage cell line (MH-S) described in Mbawuike et al., J. Leukoc. Biol. 46, 119 (1989).

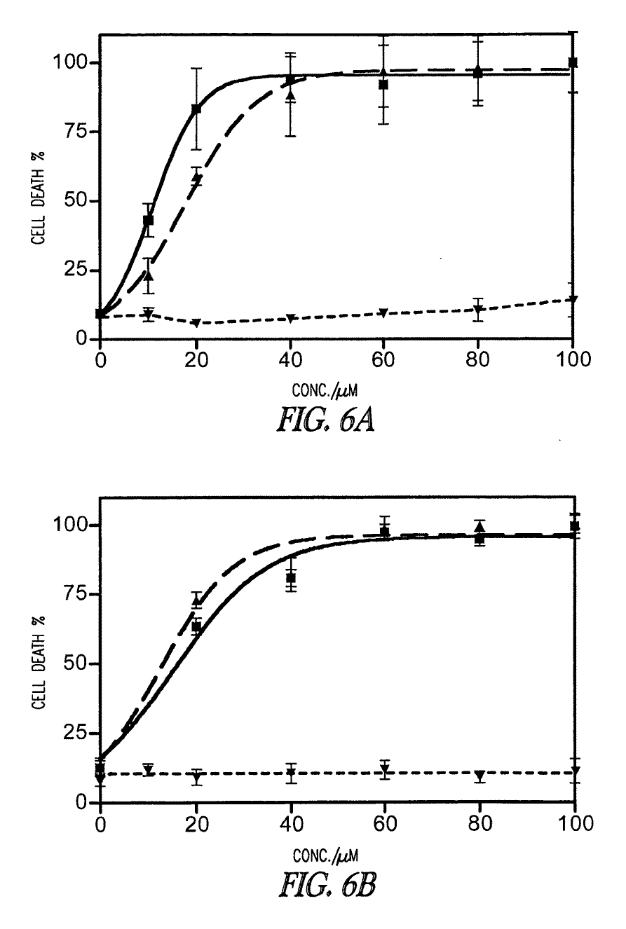

Chemically synthesized 4a and 5a are cytotoxic against a range of cell types known to be present within atherosclerotic plaque; leukocytes, vascular smooth muscle and endothelial cells. The results are shown in FIG. 6 and in Table 3.

TABLE 3

| Cell Line | IC₅₀ of 4a | IC₅₀ of 5a |
|---|---|---|
| WIL2 | 10.9 ± 1.6 μM | 17.7 ± 2.3 μM |
| Jurkat E6.1 1 | 15.5 ± 1.7 μM | 12.6 ± 1.9 μM; |
| HAEC | 24.6 ± 3.2 μM | 18.2 ± 1.9 μM |
| VSMC | 21.9 ± 2.2 μM | 29.8 ± 2.8 μM |
| J774A.1 | 15.6 ± 2.1 μM | 26.1 ± 2.8 μM |
| MH-S | 11.2 ± 1.2 μM | 13.6 ± 1.1 μM |

The IC₅₀ values of 4a and 5a are very similar against all the cells lines tested. Moreover, the cytotoxic profiles of compounds 4a and 5a against the cells lines tested were very similar. These results were surprising considering the significant structural differences between 4a and 5a. However, 4a and 5a do equilibrate with each other in a process that is facilitated by cellular components such as amino acids vide supra, 4a and 5a may be in equilibrium with each other during the time frame of the cytotoxicity assays. Hence, compounds 4a and 5a may have similar cytotoxicity in vivo.

Using similar procedures, compounds 6a, 7a, 7c, 10a, 11a and 12a have been shown by the inventors to be cytotoxic to leukocyte cell lines and the seco-ketoaldehyde 4a and its aldol adduct 5a have been shown to be cytotoxic towards neuronal cell lines.

The juxtaposition of ozone and cholesterol can lead the cytotoxic steroids 4a-6a, which generated in situ may well play a role in the progression of the lesion by promoting endothelial or smooth muscle cell damage, or by triggering apoptosis of inflammatory cells within the atheroma vide supra. Ozonolysis of cholesterol within the previously described crystalline-phase of atherosclerotic plaques may contribute to plaque destabilization, which is thought to be the ultimate step prior to arterial occlusion.

### Example 5

### Cholesterol Ozonolysis Products Promote Foam Cell Formation and Alter LDL and Apoprotein B₁₀₀ Structures

Modifications of low-density lipoprotein (LDL) that increase its atherogenicity are considered pivotal events in the development of cardiovascular disease. D. Steinberg, J. Biol. Chem. 272, 20963 (1997). For example, oxidative modifications to LDL, or apoprotein B₁₀₀ (apoB-100, the protein component of LDL), that increase LDL uptake into macrophages via CD36 and other macrophage scavenger receptors are considered critical causative pathological events in the onset of atherosclerosis. This Example describes experiments showing that cholesterol ozonolysis products 4a and 5a can promote formation of foam cells from macrophages and modify the structure of LDL and apoB-100.

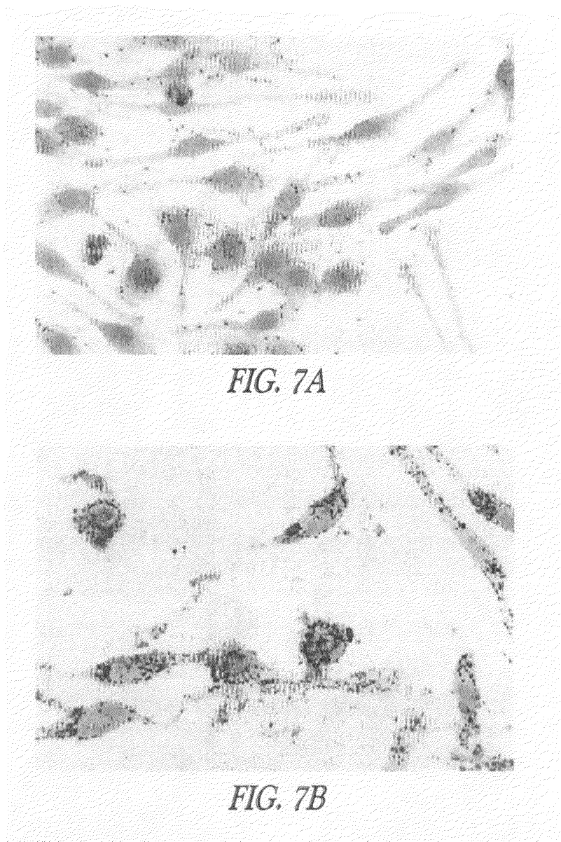

LDL (100 μg/mL) was incubated with 4a or 5a in the presence of unactivated murine macrophages (J774.1) as described in Example 1. After exposure to 4a or 5a these macrophages began lipid-loading and foam cells began to appear in the reaction vessel (FIG. 7).

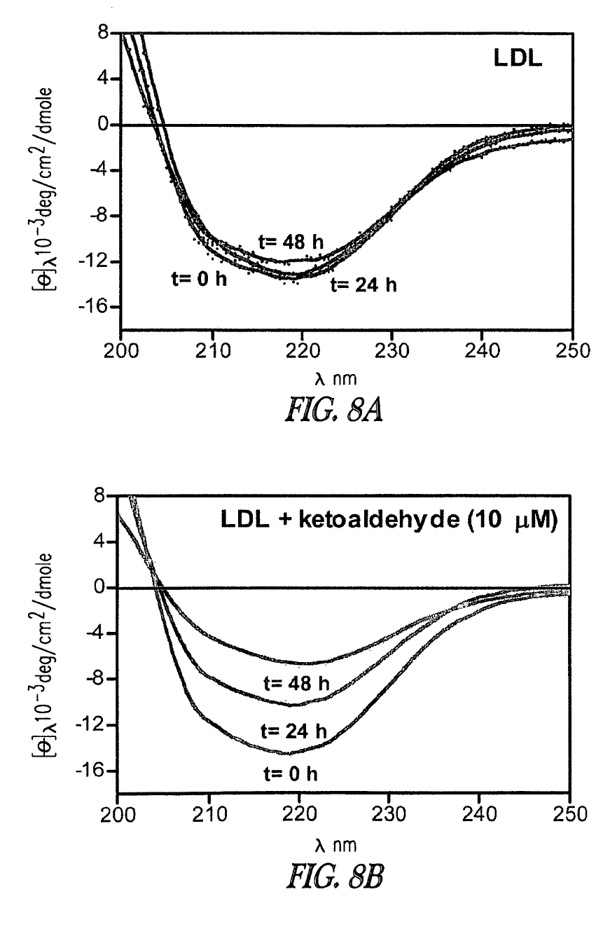

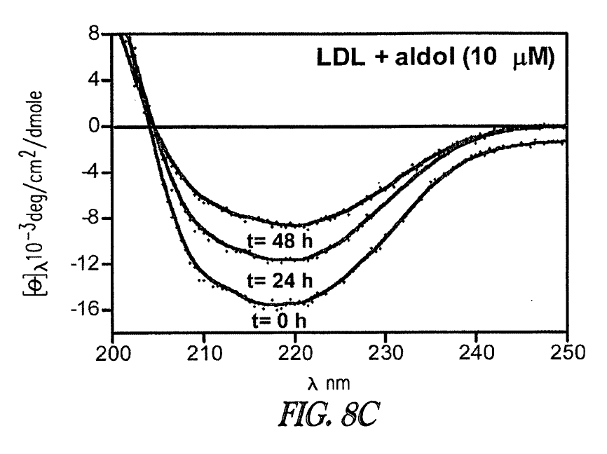

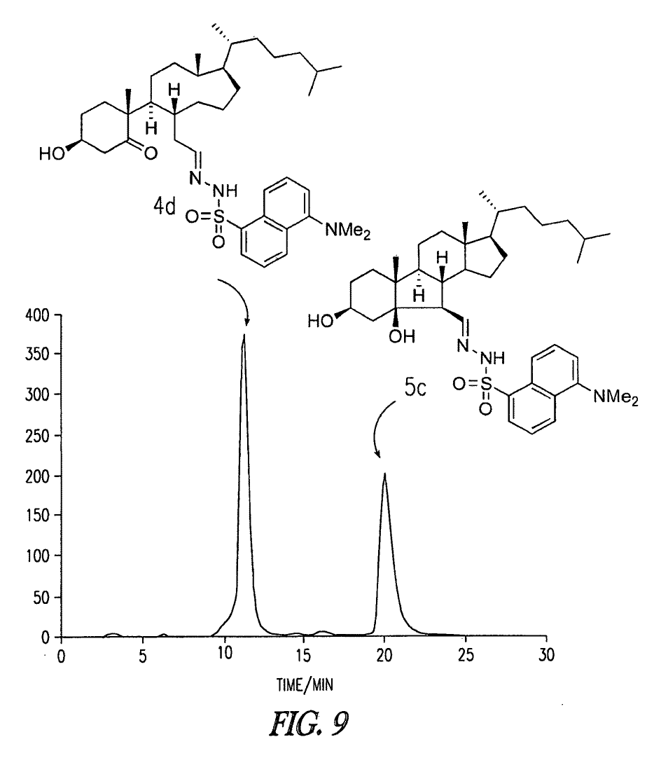

Moreover, incubation of human LDL (100 μg/ml) with 4a and 5a (10 μM) led to time-dependent changes in the structure of apoB-100 as detected by circular dichroism (FIGS. 8B,C). Circular dichroism analysis of total LDL without 4a and 5a revealed that LDL secondary structure is generally stable over the duration of the experiment (48 h) (FIG. 8A). As shown in FIG. 8A, the protein content of normal LDL has a large proportion of a helical structure (˜40±2%) and smaller amounts of β structure (˜13±3%), β turn (˜20±3%) and random coil (27±2%). However, while the spectral shape of LDL incubated with 4a and 5a remains somewhat similar to native LDL (FIGS. 8B and C), there is a significant loss of secondary structure, mainly a loss of a helical structure (4a ˜23±5%; 5a ˜20±2%) and a correspondingly higher percentage of random coil (4a ˜39±2%; 5a 32±4%). Hence, the 4a and 5a cholesterol ozonolysis products appear to undermine the structural integrity of LDL.

In order to modify LDL structure, a covalent reaction may occur between the aldehyde moieties of the 4a and 5a cholesterol ozonolysis products and the ε-amino-side-groups of apoB-100 lysine residues to form Schiff-base or enamine intermediates, that are similar to compounds previously observed in a reaction between malondialdehyde and 4-hydroxynonenal with apoB-100. Steinbrecher et al., Proc. Natl. Acad. Sci. U.S.A. 81, 3883 (1984); Steinbrecher et al., Arteriosclerosis 1, 135 (1987); Fong et al., J. Lipid. Res. 28, 1466 (1987). Such Schiff-base or enamine intermediates can have a significant lifetime and may render the derivatized LDL into a form recognized by the macrophage scavenger receptors. Hence, a covalent reaction between the 4a and 5a cholesterol ozonolysis products and apoB-100-LDL may generate a derivatized apoB-100-LDL complex that is recognized and taken up at a higher rate by macrophage scavenger receptors, thereby generating the foam cells observed in FIG. 7.

The only known oxidized forms of cholesterol that contain an aldehyde component are the 4a and 5a ozonolysis products. Hence, a reaction between such cholesterol derivatives and LDL/apoB-100 may provide a here-to-fore missing link between cholesterol, foam cell formation arterial plaque formation. Detection of high levels of the 4a and 5a ozonolysis products in the bloodstream of patients may therefore provide a direct measure of the extent to which those patients suffer from atherosclerosis.

### Example 6

### Antibodies Against Cholesterol Ozonation Products

This Example describes antibodies generated against haptens having formula 13a, 14a or 15a that can react with the ozonation and hydrazone products of cholesterol. The structures of haptens having formula 13a, 14a and 15a are shown below:

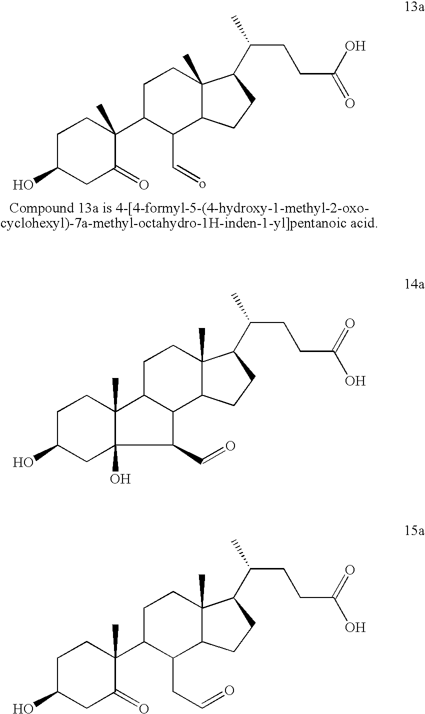

### Methods

KLH conjugates of compounds 13a, 14a and 15a were prepared. Mice were immunized with these KLH conjugates by standard procedures. Spleens were removed from the mice and dispersed to obtain splenocytes as antibody-producible cells.

The splenocytes and SP2/0-Ag14 cells, ATCC CRL-1581, derived from mouse myeloma, were co-suspended in serum-free RPMI-1640 medium (pH 7.2), pre-warmed to 37° C., to give cell densities of 3×10⁴ cells/ml and 1×10⁴ cells/ml, respectively. The suspension was centrifuged to collect a precipitate. To the precipitate, 1 ml of serum-free RPMI-1640 medium containing 50 w/v % polyethylene glycol (pH 7.2) was dropped over 1 min, followed by incubating the resulting mixture at 37° C. for 1 min. Serum-free RPMI-1640 medium (pH 7.2) was further dropped to the mixture to give a final volume of 50 ml, and a precipitate was collected by centrifugation. The precipitate was suspended in HAT medium, and divided into 200 μl aliquots each for a well of 96-well microplates. The microplates were incubated at 37° C. for one week, resulting in about 1,200 types of hybridoma formed. Supernatants from the hybridomas were analyzed by immunoassay for binding to cholesterol ozonation products.

Hybridomas KA1-11C5 and KA1-7A6, raised against a compound having formula 15a, were deposited under the terms of the Budapest Treaty on Aug. 29, 2003 with the American Type Culture Collection (10801 University Blvd., Manassas, Va., 20110-2209 USA (ATCC)) as ATCC Accession No. ATCC Numbers PTA-5427 and PTA-5428. Hybridomas KA2-8F6 and KA2-1E9, raised against a compound having formula 14a, were deposited with the ATCC under the terms of the Budapest Treaty also on Aug. 29, 2003 as ATCC Accession No. ATCC PTA-5429 and PTA-5430.

Pools of monoclonal antibody preparations KA1-7A6:6 and KA1-11C5:6, produced against a KLH conjugate of hapten 15a, and KA2-8F6 and KA2-1E9, produced against a KLH-conjugate of hapten 14a, were generated. The binding titres of the KA1-7A6:6 and KA1-11C5:6 monoclonal antibodies elicited to 15a against ozonation products 5a and cholesterol hapten 3c were determined by ELISA assay. ELISA assays were also performed to determine the binding titres of KA2-8F6:4 and KA2-1E9:4 antibodies (elicited to ozonation product 5a) against 13b, 14b and cholesterol hapten 3c.

The structure of the cholesterol hapten 3c is provided below.

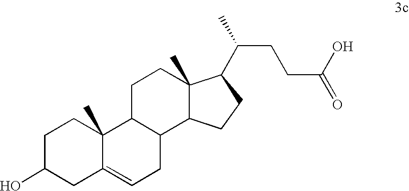

The ELISA assays were performed as follows. BSA conjugates of 13a, 14a, 3c, 13b, 14b or 15a were separately added to hi-bind 96-well microtiter plates (Fischer Biotech.) and allowed to stand overnight at 4° C. The plates were washed exhaustively with PBS and a milk solution (1% w/v in PBS, 100 μL) was added. Plates were allowed to stand at room temperature for 2 h and then washed with PBS. Cultures containing different antibody preparations were serially diluted with PBS and 50 μL of each dilution was separately added to the first well of each row. After mixing and dilution, the plates were allowed to stand overnight at 4° C. The plates were washed with PBS and a goat anti-mouse horseradish peroxidase conjugate (0.01 μg, 50 μL) was added. Plates were incubated at 37° C. for 2 h. The plates were washed and substrate solution (50 μL) 3,3′,5,5′-tetramethylbenzidine [0.1 mg in 10 mL of sodium acetate (0.1 M, pH 6.0) and hydrogen peroxide (0.01% % w/v)] was added. The plates were developed in the dark for 30 min. Sulfuric acid (1.0 M, 50 μL) was added to quench the reaction and the optical density was measured at 450 nm.

The reported titer is the serum dilution that corresponds to 50% of the maximum optical density. The data were analyzed with Graphpad Prism v. 3.0 and are reported as the mean value of at least duplicate measurements.

### Results

The results of the ELISA tests are shown in Tables 4 and 5.

TABLE 4
Binding titres of anti-15a antibodies KA1-7A6:6 and KA1 11C5:6
against 15a, ozonation product 5a and cholesterol hapten 3c.

| Antibody | 15a | 5a | 3c |
|---|---|---|---|
| KA1-7A6:6 | 32,000 | 32,000 | 16,000 |
| KA1 11C5:6 | 64,000 | 64,000 | 16,000 |

*titres were measured by ELISA against a BSA conjugate of 15a, 5a and 3c. The absolute value is the dilution factor of a tissue culture supernatant solution of antibody that corresponds to 50% of maximum absorbance when bound.

As shown by Table 4, the apparent binding affinities, measured as described above, are almost identical.

TABLE 5
Binding titres of KA2-8F6:4 and KA2-1E9:4 antibodies elicited
to 5a against 15b, 14b and cholesterol hapten 3c.

| antibodies | 15b | 14b | 3c |
|---|---|---|---|
| KA2-8F6:4 | 32,000 | 32,000 | 16,000 |
| KA2-1E9:4 | 64,000 | 64,000 | 16,000 |

*titres were measured by ELISA against a BSA conjugate of 15b, 14b and cholesterol hapten 3c. The absolute value is the dilution factor of a tissue culture supernatant solution of antibody that corresponds to 50% of maximum absorbance when bound to a BSA conjugate of 13b, 15b and cholesterol hapten 3c.

These results indicate that high affinity antibody preparations can be generated against cholesterol ozonation products.

### Example 7

### Additional Methods for Detecting Cholesterol Ozonation Products

This Example illustrates that cholesterol ozonation products can be detected by a variety of procedures, including by conjugation of the free aldehyde groups on these ozonation products to fluorescent moieties and by use of antibodies reactive with these ozonation products.

### Materials and Methods

### General Methods

All reactions were performed with dry reagents, solvents, and flame-dried glassware unless otherwise stated. Starting materials were purchased and used as received from Aldrich Chemical Company, unless otherwise stated. Cholesterol-[26,26,26,27,27,27-D₆] was purchased from MEDICAL ISOTOPES, INC. Flash column chromatography was performed using silica gel 60 (230-400 mesh). Cholesterol ozonation products 4a and 5a and the 2,4-dinitrophenyl hydrazones of ozonation products 4a and 5a (4b and 5b, respectively) were synthesized as described in the previous examples. Thin layer chromatography (TLC) was performed using Merck (0.25 mm) coated silica gel Kieselgel 60 F₂₅₄ plates and visualized with para-anisaldehyde stain. ¹H NMR spectra were recorded on Bruker AMX-600 (600 MHz) spectrometer. ¹³C NMR spectra were recorded on Bruker AMX-600 (150 MHz) spectrometer. Chemical shifts are reported in parts per million (ppm) on the 6 scale from an external standard.

### Dansyl Hydrazone of 3β-hydroxy-5-oxo-5,6-secocholestan-6-al (4d)

Dansyl hydrazine (50 mg, 0.17 mmol) and p-toluenesulfonic acid (1 mg, 0.0052 mmol) was added to a solution of cholesterol ozonation product 4a (65 mg, 0.16 mmol) in acetonitrile (8 ml). The reaction mixture was stirred under an argon atmosphere for 2 h at room temperature, and evaporated to dryness in vacuo. The residue was dissolved in methylene chloride (10 ml) and washed with water (2×10 ml). The organic fraction was dried over magnesium sulfate and concentrated in vacuo. The crude yellow oil was purified by silica gel chromatography [ethyl acetate-hexane (1:1; 7:3)] to give the title compound 4d (70 mg, 68%) as a mixture of geometric isomers (cis:trans 8:92): ¹H NMR (CDCl₃) δ 9.341 (s, 1H), 8.567 (d, J=8.4 Hz, 1H), 8.358 (dd, J=7.2, 1.2 Hz, 1H), 8.290 (d, J=8.4 Hz, 1H), 7.550 (dd, J=8.4, 7.6 Hz, 1H), 7.539 (dd, J=8.4, 7.6 Hz, 1H), 7.167 (d, J=7.6 Hz, 1H), 7.000 (t, J=4.0 Hz, 0.92H trans), 6.642 (dd, J=6.8, 2.8 Hz, 0.08H cis), 4.273 (bs, 1H), 3.045 (dd, J=13.6, 3.4 Hz, 1H), 2.869 (s, 6H), 2.233 (d, J=13.6 Hz, 1H), 2.097 (dt, J=18, 4.4 Hz, 1H), 1.162 (s, 3H), 0.904 (d, J=6.4 Hz, 3H), 0.899 (d, J=6.8 Hz, 3H), 0.892 (d, J=6.4 Hz, 3H), 0.513 (s, 3H); ¹³CNMR (CDCl₃) δ 209.66, 151.77, 149.49, 133.52, 131.20, 130.99, 129.64 (2C)*, 128.52, 123.25, 118.83, 115.25, 71.07, 56.20, 52.68, 52.56, 47.10, 45.40, 42.32, 40.81, 39.82, 39.48, 36.51, 36.05, 35.79, 34.39, 31.05, 28.02, 27.74, 27.30, 24.27, 24.13, 22.99, 22.84, 22.56, 18.53, 17.45, 11.31; HRMALDIFTMS calcd for C₃₉H₅₉N₃O₄SNa (M+Na) 688.4118, found 688.4152; R_{f} 0.43 [ethyl acetate-hexane (7:3)]. *2C denotes that this signal is believed to correspond to two carbon signals (C₀ as per gHSQC) from the dansyl moiety.

### Dansyl hydrazone of 3β-Hydroxy-5β-hydroxy-B-norcholestane-6β-carboxaldehyde (5c)

To a solution of cholesterol ozonation product 5a (30 mg, 0.072 mmol) in tetrahydrofuran (5 ml) was added dansyl hydrazine (25 mg, 0.08 mmol) and hydrochloric acid (conc., 0.05 ml). The white precipitate that immediately formed was dissolved by the addition of water (0.2 ml). The homogeneous reaction mixture was stirred under an argon atmosphere for 3 h at room temperature, and evaporated to dryness. The red residue was dissolved in ethyl acetate (10 ml) and washed with water (2×10 ml). The organic fraction was dried over magnesium sulfate and concentrated in vacuo. The crude yellow oil was purified first by silica gel chromatography [ethyl acetate-methylene chloride (1:4-1:1)] and then by preparative HPLC (C18 Zorbax 21.22 mm and 25 cm. 100% acetonitrile) to give the title compound 5c (14.5 mg, 30%) as a mixture of geometric isomers (cis:trans 17:83): ¹H NMR (CDCl₃) δ 8.557 (d, J=8.8 Hz, 1H), 8.372 (dd, J=7.2, 1.2 Hz, 1H), 8.300 (d, J=8.8 Hz, 1H), 8.084 (s, 1H), 7.575 (dd, J=8.8, 7.6 Hz, 1H), 7.554 (dd, J=8.8, 7.6 Hz, 1H), 7.197 (d, J=7.6 Hz, 1H), 7.057 (d, J=7.2 Hz, 0.84H trans), 6.517 (d, J=5.2 Hz, 0.16H cis), 4.229 (m, 0.17H cis), 4.004 (m, 0.83H trans), 2.905 (s, 6H), 2.379 (bm, 4H), 1.913 (dd, J=9.6, 7.2 Hz, 2H), 0.886 (d, J=6.8 Hz, 3H), 0.879 (d, J=6.4 Hz, 3H), 0.841 (d, J=6.8 Hz, 3H), 0.691 (s, 3H), 0.393 (s, 3H); ¹³C NMR (CDCl₃) δ 154.081, 133.425, 131.367, 130.912, 129.695, 128.611, 123.350, 115.121, 83.268, 70.469, 67.079, 55.773, 55.677, 55.280, 51.652, 45.429, 45.038, 44.372, 43.129, 42.443, 39.488, 36.143, 35.585, 28.580, 28.458, 27.984, 27.766, 23.850, 22.825, 22.549, 21.389, 18.659, 18.063, 12.192; HRMALDIFTMS calcd for C₃₉H₅₉N₃O₄SNa (M+Na) 688.4118, found 688.4118; R_{f} 0.41 [ethyl acetate-methylene chloride (1:1)].

### 3β-Hydroxy-5-oxo-5,6-seco-[26,26,26,27,27,27-D₆]-cholestan-6-al (D6-4-a)

A gaseous mixture of ozone in oxygen was bubbled through a solution of D₆-cholesterol (50 mg, 0.13 mmol) in 5 mL chloroform-methanol (9:1) at −78° C. for 1 min, by which time the solution turned slightly blue. The reaction mixture was evaporated and stirred with Zn powder (40 mg, 0.61 mmol) in 2.5 mL acetic acid-water (9:1) for 3 h at room temperature. This heterogeneous mixture was diluted with methylene chloride (10 mL) and washed with water (3×5 mL) and brine (5 mL). The organic fractions were dried over magnesium sulfate and evaporated. The residue was purified using silica-gel chromatography (eluted with hexane-ethyl acetate 5:1, 3:1 and 2:1) to yield the title compound as a white solid (44 mg, 0.104 mmol), yield: 81%. ¹H NMR 600 MHz (δ, ppm, CDCl₃): 9.61 (s, 1H), 4.47 (s, 1H), 3.09 (dd, 1H, J=13.6 Hz, 4.0 Hz), 2.25-2.40 (m, 3H), 2.15-2.19 (m, 1H), 1.01 (s, 3H), 0.88 (d, 3H, J=6.1 Hz), 0.67 (s, 3H). ¹³C NMR 150 MHz (δ, ppm, CDCl₃): 217.5, 202.8, 71.0, 56.1, 54.2, 52.6, 46.8, 44.1, 42.5, 42.1, 39.8, 39.3, 35.9, 35.7, 34.7, 34.0, 27.8, 27.7, 27.5, 25.3, 23.7, 23.0, 18.5, 17.5, 11.5.

3β-hydroxy-5βhydroxy-B-norcholesterol-[26,26,26,27,27,27-D₆]-6β-carboxaldehyde (D₆-5a). To a solution of D₆-4-a (26 mg, 0.061 mmol) in acetonitrile-water (20:1, 5 mL) was added L-proline (11 mg). The reaction mixture was stirred for 2.5 h at room temperature and evaporated in vacuo. The residue was dissolved in ethyl acetate (10 mL) and washed with water (2×5 mL) and brine. The organic fraction was dried over magnesium sulfate and evaporated to leave a white solid which was analytically pure (26 mg, 0.061 mmol, yield: 100%), for NMR. ¹H NMR 600 MHz (δ, ppm, CDCl₃): 9.69 (s, 1H), 4.11 (s, 1H), 2.23 (dd, 1H, J=9.2 Hz, 3.0 Hz), 0.91 (s, 3H), 0.90 (d, 3H, J=6.6 Hz), 0.70 (s, 3H); ¹³C NMR 150 MHz (δ, ppm, CDCl₃): 204.7, 84.2, 67.3, 63.9, 56.1, 55.7, 50.4, 45.5, 44.7, 44.2, 40.0, 39.7, 39.3, 36.1, 35.6, 28.3, 27.9, 27.5, 26.7, 24.5, 23.8, 21.5, 18.7, 18.4, 12.5.

4-(5-(4-hydroxy-1-methyl-2-oxocyclohexyl)-7α-methyl-4-(2-oxoethyl)-octahydro-1H-inden-1-yl)pentanoic acid 15a. Ozonolysis of 3β-hydroxycholest-5-en-24-oic acid 3c, was performed as described for D₆-5a. ¹H NMR 400 MHz (δ, ppm, CDCl₃): 9.60 (s, 1H); 4.47 (s, 1H), 3.40 (dd, J=13.6 Hz, 4 Hz, 1H); 1.00 (s, 1H), 0.91 (d, J=6.4 Hz, 3H), 0.67 (s, 3H). ¹³C NMR 100 MHz (δ, ppm, CDCl₃): 218.7, 202.9, 179.8, 70.9, 55.5, 54.1, 52.5, 46.4, 44.0, 42.4, 42.1, 39.6, 35.1, 34.5, 34.0, 30.8, 30.4, 27.5, 27.3, 25.1, 22.8, 17.9, 17.4, 11.4.

### Cholesterol Ozonation Product Extraction.

A modified Bligh and Dyer method was used to extract total lipids from both blood and tissue samples. See, Bligh E G, D. W. Can J Biochem Physiol 1959, 37, 911-17. Human plasma (200 μL), collected in Vacutainer tubes, containing citrate or EDTA as anticoagulant and stored at 4° C., was added to potassium dihydrogen phosphate (KH₂PO₄, 0.5 M, 300 μL) in a capped glass tube. Methanol (500 μL) was added and the sample was vortexed briefly. Chloroform (1 mL) was added and the sample was vortexed for 2 min, centrifuged at 3000 rpm for 5 min and the organic layer was removed. This process of chloroform addition, vortexing and centrifugation was repeated. The combined organic fractions were combined and evaporated in vacuo. Endarterectomy specimens were obtained from patients undergoing carotid endarterectomy for routine indications. The Scripps Green Hospital Institutional Review Board approved the human subjects protocol. Specimens were frozen and stored at −70° C. prior to analysis. For analysis, the tissue sample was allowed to warm to room temperature and was then homogenized in aqueous buffer (KH₂PO₄, 0.5M, 1-2 mL) using a tissue homogenizer (Tekmar). The homogenate was added to a solution of methanol:chloroform (1:3, 6 mL) and centrifuged at 3000 rpm for 5 min. The organic fraction was collected. Chloroform (6 mL) was added to the remaining aqueous miscible fraction and the samples were centrifuged (3000 rpm for 5 min). The combined organic fractions were then evaporated in vacuo.

Derivatization with Dansyl Hydrazine and HPLC-Analysis of Extracted Cholesterol Ozonation Products.

The evaporated blood or tissue extracts vide supra are resuspended in isopropanol (200 μL) containing dansyl hydrazine (200 μM) and H₂SO₄ (100 μM) and incubated at 37° C. for 48 h. The analytical method involved HPLC analysis on a Hitachi D-7000 HPLC system connected to a Vydec C-18 RP column with an isocratic mobile phase of acetonitrile:water (90:10, 0.5 mL/min) using fluorescence detection (Excitation wavelength 360 nm, Emission wavelength 450 nm). The retention time (R_{T}) for the dansyl derivative of ozonation product 5a (5c) was about 8.1 min. The retention time for the hydrazine derivative of 5a (5b) was about 10.7 min. Concentrations were routinely determined by peak area calculations referenced to authentic standards using a Macintosh PC and Prism 4.0 software.

### Gas Chromatography—Mass Spectroscopy

Evaporated specimens were reconstituted in methylene chloride to a 1 mL volume and silylated by the addition of 100 uL pyridine and 100 uL N,O-Bis(trimethylsilyl)-trifluoroacetamide with 1% trimethylchlorosilane to the concentrated plaque extract. Samples were incubated at 37° C. for 2 hours then evaporated to dryness by rotatory evaporation. Each sample was resuspended in 100 uL methylene chloride prior to analysis. 2.5 ul of sample was injected via a splitless injection (Agilent 7673 autosampler) onto an HP-5 ms column, 30m×0.25 mm ID×0.25 um film thickness, flow rate of 1.2 ml/min, injector temp was 290° C., temperature program starts at 50° C., hold for 5 min then ramp at 20° C./min until 300° C., hold for 12 min. Mass Analysis was performed with an Agilent model 5973 inert, Scan range 50-700 m/z followed by selected ion monitoring (SIM) scans for m/z 354 and 360. MS quad temp was 150° C., with an MS source temp of 280° C.

### Coupling of Hapten 15a to Carrier Proteins KLH and BSA.

1-Ethyl-3,3′-dimethylaminopropyl-carbodiimide hydrochloride (EDC, 1.5 mg, 0.008 mmol) and Sulfo N-hydroxysuccinimide (1.8 mg, 0.008 mmol) were dissolved in 0.01 mL H₂O and added to a solution of hapten (2.5 mg, 0.006 mmol) in 0.1 mL DMF. The mixture was vortexed and kept at room temperature for 24 hours before it was added to BSA (5 mg) in PBS buffer (0.9 ml, 0.05 mM at pH=7.5) at 4° C. This final mixture was kept at 4° C. for 24 hours and stored at −20° C. The reactions involved in synthesizing a KLH or BSA conjugate of compound 15a are depicted below.

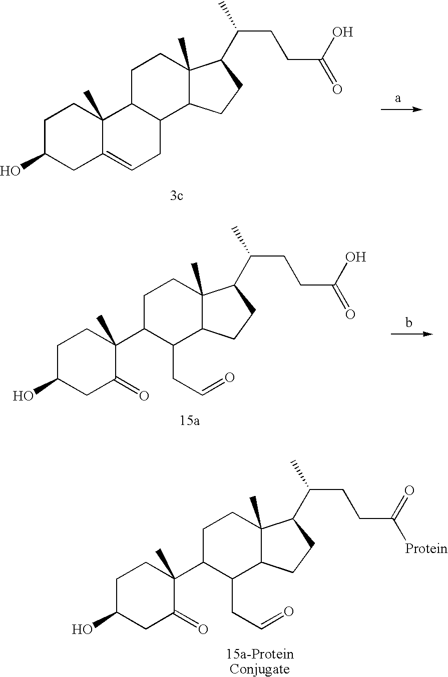

Reaction a involved ozonolysis of compound 3c with O₃/O₂ as described above. Reaction b involved treatment of compound 15a with EDC and HOBt in DMF overnight followed by incubation with BSA or KLH in phosphate buffered saline (PBS), pH 7.4.

Monoclonal antibody production was carried out by standard methods. Immunization of 8 week old 129GIX+mice was performed with 10 ug KLH-15a conjugate in 50 uL PBS per mouse mixed with an equal volume of RIBI adjuvant injected IP every 3 days for a total of 5 immunizations. The serum titer was determined by ELISA. 30 days later, a final injection of 50 ug KLH-15a conjugate in 100 uL PBS intravenously (IV) in the lateral tail vein. Animals were sacrificed and the spleen was removed 3 days later for fusion. Spleen cells from immunized animals were mixed 5:1 with X63-Ag8.653 myeloma cells in RPMI media centrifuged, and resuspended in 1 mL PEG 1500 at 37 C The PEG is diluted with 9 mL RPMI over 3 minutes and incubated at 37 C for 10 minutes then centrifuged, resuspended in media and plated in 15×96 well plates. ELISA was performed to screen for antibodies that bound cholesterol ozonation product 4a or 5a but not cholesterol. Selected hybridomas were subcloned through 2 generations to guarantee monoclonality.

Preparations of Histological Sections from Ascending Aorta of ApoE Knockout Mice.

Specimens were snap frozen in liquid nitrogen. 10 micron sections were taken, and mounted on glass slides. Specimens were fixed by sequential immersion in 1:1 ethyl alcohol:diethyl ether for 20 minutes, 100% ethanol for 10 minutes, and 95% ethanol for 10 minutes. After washing in PBS, a 1:200 dilution of antibody specific for cholesterol ozonation product was applied and incubated with the tissue for 1 hour. Secondary labeling was performed with a 40:1 dilution of FITC labeled goat anti-mouse IgG (Calbiochem). Images were obtained using an optronics microfire digital camera and processed using Adobe Photoshop.

### Results

### Fluorescence-Detection of Dansyl Hydrazones of Cholesterol Ozonation Products.

As described in the previous Examples, cholesterol ozonation products can be detected in vivo using a modification of the analytical procedure developed in a chemical study by K. Wang, E. Bermudez, W. A. Pryor, Steroids 58, 225 (1993). This modified process involved extraction of a suspension of the homogenized plaque material (˜50 mg wet weight) in PBS (1 mL) pH 7.4, into an organic solvent (methylene chloride, 3×5 mL) treatment of the organic soluble fraction with an ethanolic solution of 2,4-dinitrophenylhydrazine hydrochloride (DNPH HCl) (2 mM, pH 6.5) for 2 h at room temperature. This reaction mixture was analyzed by reversed-phase HPLC (direct injection, u.v. detection at 360 nm) and in-line negative ion electrospray mass-spectroscopy for the presence of 4b, the 2,4-dinitrophenylhydrazone (2,4-DNP) derivative of 4a and 5b, the 2,4-DNP derivative of 5a. This technique is both rapid and highly sensitive. However, there are a number of limitations to this assay when it is applied to biological samples. These include interference with other biologic compounds with ultraviolet absorbance at 360 nm, conversion of the 4b into 5b during the conjugation reaction, and the reduced efficiency of the conjugation reaction at low concentrations of cholesterol ozonation products.

Therefore, a new procedure was tested to ascertain whether increased assay sensitivity could be achieved. This procedure involved conjugation of cholesterol ozonation products to a hydrazine that had a fluorescent chromophore followed by fluorescence detection and HPLC analysis. The fluorescent chromophore selected was the dansyl group. The assay involved derivatization of the extracted cholesterol ozonation products with dansyl hydrazine under acidic conditions as described above. The product of dansyl hydrazine reaction with cholesterol ozonation product 4a was 4d, which is depicted below.

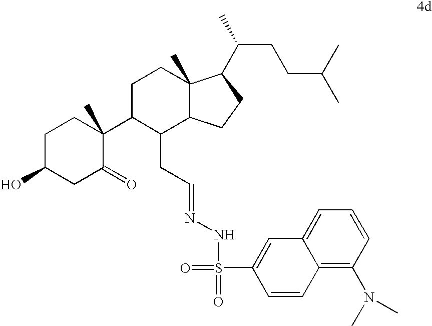

The product of dansyl hydrazine reaction with cholesterol ozonation product 5a was 5c, which is depicted below.

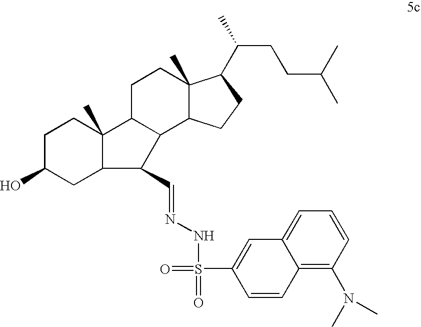

The reaction efficiency for dansyl hydrazine derivatization was evaluated in a range of solvents, such as hexanes, methanol, chloroform, tetrahydrofuran, acetonitrile, and isopropanol (IPA). From this analysis, it was determined that IPA was the optimal solvent in terms of reaction efficiency and lowest rate of spontaneous aldolization of cholesterol ozonation product 4a to 5a. The reaction efficiency was quantified by HPLC using chemically synthesized authentic dansyl hydrazone standards 4d and 5c (FIG. 9). The derivatization efficiency for cholesterol ozonation product 4a with dansyl hydrazine (200 μM) and sulfuric acid (100 μM) in IPA at 37° C. for 48 h, to form 4a hydrazone derivative 4d with a retention time (R_{T}) of about 11.2 min, was 86.0±8.0%. Importantly, only 1.3% of 5c was formed by aldolization of 4a or 4d during the derivatization process. The efficiency of conversion of 5a into its dansyl hydrazone derivative 5c (R_{T}˜19.4 min) was 83±11% for a concentration range of 5a from 0.01-100 μM. The level of sensitivity for the dansyl-hydrazones 4d and 5c is ˜10 nM.

To determine the efficiency by which the 4a and 5a cholesterol ozonation products are extracted and derivatized from plasma samples, human plasma samples were spiked with 5a and then extracted and conjugated with either 2,4-DNP or dansyl hydrazine. There was no significant difference in the amount of conjugated hydrazone detected with either method; 37.5±1.9% derivatized as the dansyl hydrazone 5c and 31±8.9% recovered as 2,4-DNP hydrazone 5b. Isotope dilution-gas chromatography with in-line mass spectrometry (ID-GCMS).

At present, most analytical methods for the determination of oxysterols in cholesterol-rich tissues, such as blood (plasma) and atherosclerotic arteries are based on GC with flame ionization detection (FID) or selected ion monitoring (SIM). The advantage of SIM over FID methods is the specificity of detection. This specificity is required for the analysis of oxysterols in biological matrices. The critical aspect to the SIM strategy is the use of internal standards. The most common being 5α-cholestane. See, Jialil, I.; Freeman, D. A.; Grundy, S. M. Aterioscler. Thromb. 1991, 11, 482-488; Hodis, H. N.; Crawford, D. W.; Sevanian, A. Atherosclerosis 1991, 89, 117-126. However, GC-MS with deuterium-labeled internal standards is the preferred method because it is sensitive and specific and corrects for the different recovery of different analytes. Dzeletovic, S.; Brueuer, O.; Lund, E.; Diszfalusy, U. Analytical Biochem. 1995, 225, 73-80. The role of the deuterated internal standards is two-fold. First, they allow quantification by allowing a correlation of isotope abundance with concentration. Second, the addition of a known amount of the deuterated molecule prior to the extraction procedure allows an assessment of the efficiency with which the cholesterol ozonation products are being extracted. Leoni, V.; Masterman, T.; Patel, P.; Meaney, S.; Diczfalusy, U.; Bjørkhelm, I. J. Lipid. Res. 2003, 44, 793-799.

Hexadeuterated cholesterol ozonation products D₆-4a and D₆-5a were prepared from [26, 26, 26, 27, 27, 27-D]-cholesterol (deuterated 3c) as outlined below.

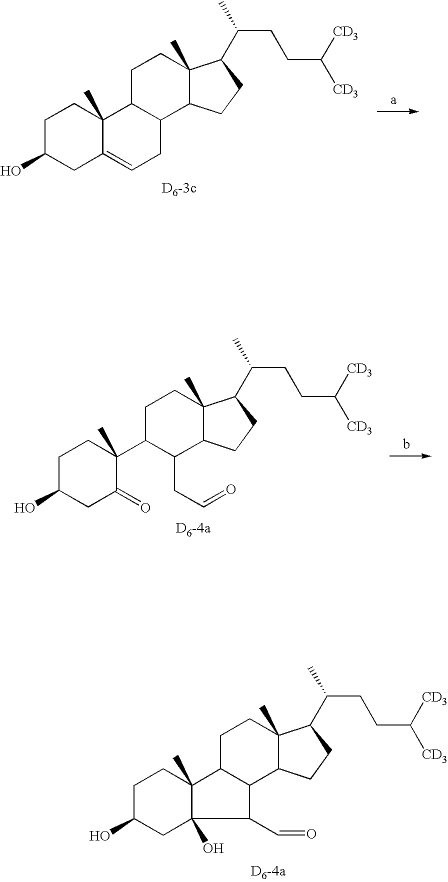

In the first step (a) of the synthesis, ozone was bubbled through a solution of D₆-3c in chloroform-methanol (9:1) at 78° C. to generate D₆-4a. In a second step (b), D₆-4a was dissolved in DMSO and reacted with proline for 2.5 hours at room temperature to generate D₆-5a.

D₆-4a and D₆-5a were used as internal standards to test the sensitivity of the GC/MS method on an in-house Agilent GC/MS. In a typical procedure, samples of authentic cholesterol, 4a, 5a, D₆-cholesterol, D₆-4a and D₆-5a were converted into their trimethylsilylethers by treatment with pyridine and BSTFA under argon at 37° C. for 2 h. After removal of the volatiles (in vacuo) the residue was dissolved in methylene chloride and transferred to an autosampler vial.

GC-MS was then performed on an Agilant Technologies 6890 GC (with a split/splitless inlet system and a 7683 autoinjector module) coupled to a 5973 Inert MSD. The mass spectrometer was operated in the full ion scan mode. The observed retention times (R_{T}) and M⁺ ions were as follows ozonation products 4a and 5a (R_{T}=29.6 min, M⁺ 354); D₆-4a and D₆-5a (R_{T}=29.6 min, M⁺ 360); cholesterol (R_{T}=27.2 min, M⁺ 329), D₆-cholesterol (R_{T}=27.2 min, M⁺ 335). The deduced fragmentation of cholesterol ozonation products 4a and 5a within the GC-MS is shown below.

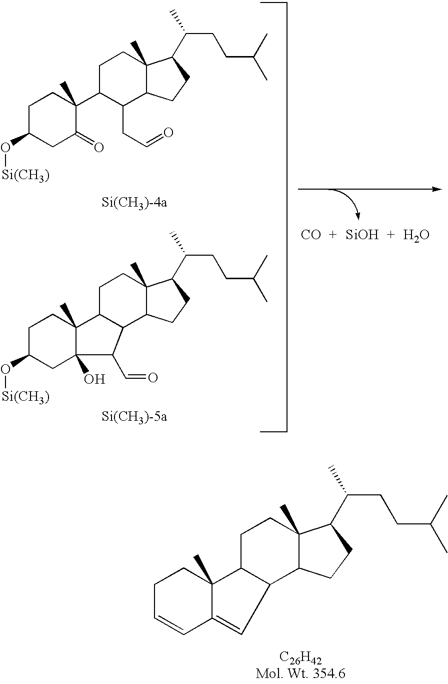

As indicated above, both cholesterol ozonation product 4a and 5a give rise to a fragment of about M⁺ 354. The deuterated (D₆) 4a and 5a cholesterol ozonation products rise to a fragment of about M⁺ 360.

Thus, no distinction between cholesterol ozonation products 4a and 5a was observed in the GC-MS assay, probably because cholesterol ozonation product 4a is converted into 5a during the silylation step. Thus, the amount of M+354 (or 360) is a measure of the concentration of authentic 4a and 5a cholesterol ozonation product. The area of the 354 ion peak is linear with concentration and the lower-level of sensitivity measured thus far is 10 fg/μL for the cholesterol ozonation products (equivalent to an estimated 2-log increase in detection limit from the LC/MS assay described in previous examples).

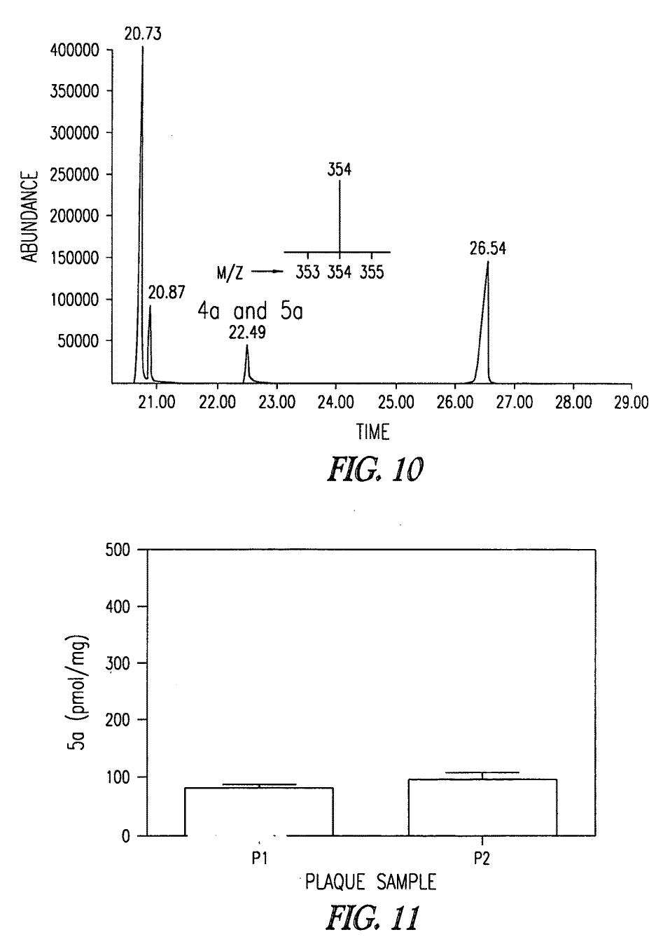

The GCMS assay was further validated by extraction of cholesterol ozonation products from clinically excised carotid plaque material. Carotid endarterectomy tissue (n=2) that had been obtained from patients undergoing carotid endarterectomy for routine analysis were homogenized using a tissue homogenizer for 10 min (under argon) and then extracted into CHCl₃/MeOH. The extract was silylated as described vide supra and then subjected to GC-MS analysis (FIGS. 10 and 11). The GC-MS trace of ion-abundance versus time shows the presence of many oxysterols that have yet to be defined. However, there was clear resolution of the combined ozonation products 4a and 5a (R_{T}=22.49 min).

These data clearly establish the feasibility of the overall extraction and GC-MS assay for the analysis of the 4a and 5a cholesterol ozonation products in biological samples and validate the results described on analysis of atherosclerotic plaque material in previous Examples.

Immunohistochemical Localization of Cholesterol Ozonation Products 4a-5a.

As described above, mice were immunized with a KLH-conjugate of compound 15a, which is an analog of cholesterol ozonation product 4a. Monoclonal antibodies were generated by hybridoma methods. Two murine monoclonal antibodies, 11C5 and 7A7 with good binding affinity <1 μM for cholesterol ozonation product 5a and excellent specificity over cholesterol (1000 fold less affinity).

Generation of an anti-5a antibody to a hapten that is a 4a analog was not too surprising because, as shown above, addition of cholesterol ozonation product 4a to blood results in its immediate conversion into 5a.

Immunohistochemical staining of frozen fixed sections of aorta from ApoE deficient mice with antibody 11C₅ and a FITC-labeled anti IgG secondary antibody demonstrated localization of cholesterol ozonation product 5a in areas of atherosclerosis within subintimal layers of the vessel when compared with consecutive sections stained with non-specific murine antibodies. Absorption of the antibody with soluble cholesterol did not eliminate the subintimal fluorescence.

### Example 8

### Further Proatherogenic Effects of Cholesterol Ozonolysis Products

This Example provides data showing that cholesterol ozonolysis products 4a and 5a are chemotactic agents that promote recruitment of macrophages to sites when LDL is present. This Example also shows that cholesterol ozonolysis products 4a and 5a up-regulate expression of Class A scavenger receptor (SR-A) in macrophages when LDL is present, and also up-regulate expression of E-selectin in endothelial cell adhesion molecule. Furthermore, this Example shows that ozonolysis product 5a (but not 4a) induced monocyte differentiation into macrophages.

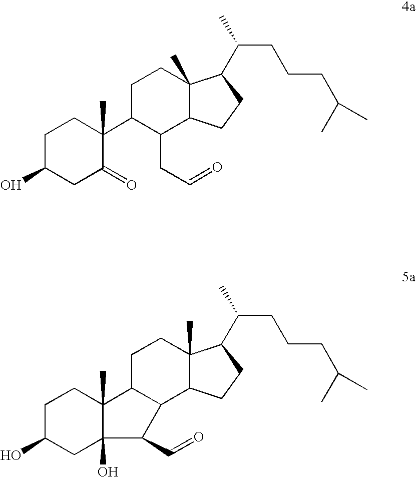

### Materials and Methods:

Unless otherwise stated, all reactions were performed under an inert atmosphere with dry reagents, solvents, and flame-dried glassware. All starting materials were purchased from Aldrich, Sigma, Fisher, or Lancaster and used as received. All flash column chromatography was performed using silica gel 60 (230-400 mesh). Preparative thin layer chromatography (TLC) was performed using Merck (0.25, 0.5, or 1 mm) coated silica gel Kieselgel 60 F254 plates. cholesterol ozonolysis product 4a (3β-Hydroxy-5-oxo-5,6-secocholestan-6-al) and cholesterol ozonolysis products 5a (3β-Hydroxy-5β-hydroxy-B-norcholestane-6β-carboxaldehyde) were synthesized as described above.

Oxidized low density lipoprotein (ox-LDL) was produced by dialyzing LDL (CalBiochem, La Jolla, Calif.) in 5 μM CuSO₄ against phosphate buffered saline (PBS), pH 7.4 at 37° C., for 12 h. Lipid peroxidation was assessed by determining the formation of thiobarbituric acid-reactive substances. The reaction mixture was treated with 2 parts 3.8% (w/v) trichloroacetic acid, 0.1% 2-thiobarbituric acid in 0.06 N HCl, and 0.2 parts 0.625 mM SDS, and then heated to 95° C. for 30 min. After cooling, the sample was extracted twice with 100 μL n-butanol and absorbance of the extract measured at 532 nm. Malondialdehyde bis(dimethyl acetal), which yields malondialdehyde by acid treatment, was used as a standard.

### Cell Culture:

HAAE-1 human aortic endothelial cell line (CRL-2472), J774A.1 murine tissue macrophage cell line (TIB-67), and monocytic cell lines U-937 histiocytic lymphoma (CRL-1593.2) and THP-1 acute monocytic leukemia (TIB-202) were obtained from ATCC. See Nichols et al. (1987) J. Cell. Physiol. 132: 453-62; Ralph et al. (1976) J. Exp. Med. 143: 1528-33; Tsuchiya et al. (1982) Cancer Res. 2: 1530-36; Sundstrom, C. & Nilsson, K. (1976) Int. J. Cancer 17: 565-77. All cell lines were cultured in ATCC recommended media with 10% fetal calf serum (FCS). Cells were incubated in a controlled atmosphere at 37° C., with 5 or 7% CO₂. The release of lactate dehydrogenase from cells was used to measure the cytotoxicity of cholesterol ozonation products as described in Example 1.

### Cholesterol Ozonation Product Localization:

J774A.1 cells were plated in 8-well chambered slides (Nalge Nunc International). Cells were gently washed twice with PBS, pH 7.4 before 5 min or 1 h exposure to 50 μM dansyl derivative of cholesterol ozonation product 5d or 5e, dansyl cholesterol derivative 9c or 9d, or dansyl hydrazine diluted in media without FCS (0.05% v/v final DMSO concentration).

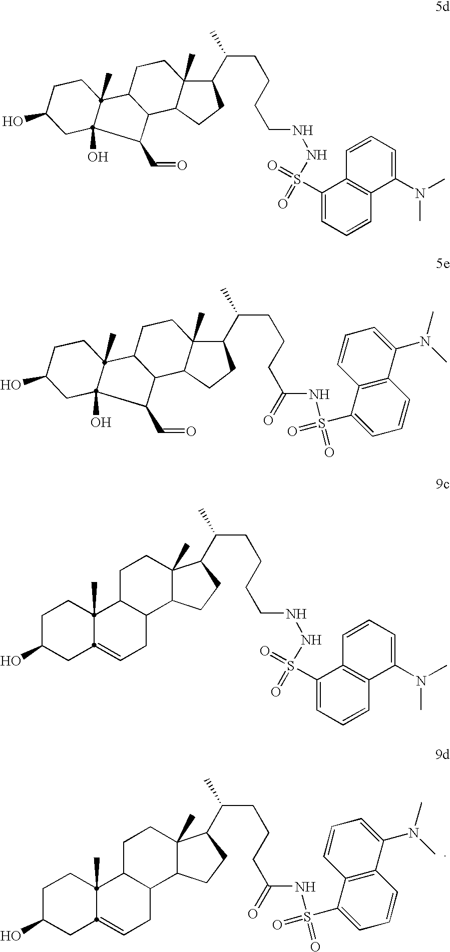

Media was removed by aspiration and cells were fixed immediately in 95% ice cold methanol for 5 min. Cells were mounted in glycerol containing Antifade reagent (Molecular Probes). Fluorescence images were documented using DeltaVision Deconvolution microscope (API, Issaquah Wash.) equipped with a Photometrics CH350L liquid cooled CCD camera attached to an Olympus IX_{—}70 inverted microscope. These data were collected using a 60× oil immersion objective lens (NA 1.4) and a filter set combination (Ex) DAPI 360/40 and (Em) 457/50. All images were deconvolved using constrained iterative algorithms (10 iterations) of DeltaVision software (softWoRx, v2.5). The deconvolved images were subsequently processed using softWoRx, v2.5.

In some experiments, murine macrophage RAW 264.7 (ECACC 91062702) cells were grown in RPMI (Gibco) with 10% FCS at 37° C. with 5% CO₂. For fluorescence imaging, cells were incubated on cover slips in 6-well chambered plates (Costar) for 12 h, gently washed 3 times in PBS at pH 7.4, and incubated with 50 μM dansyl derivative of cholesterol ozonation product 5d or 5e, dansyl cholesterol derivative 9c or 9d, in RPMI with 10% FCS for 5, 15, 30, and 60 min. A solution of the dansyl derivative was then added in DMSO [final DMSO, 0.05% (v/v)]. Media was removed by aspiration, and cells were washed twice with PBS, followed by fixing in paraformaldehyde (3.7% in PBS) for 10 min. Cover slips were mounted in MOWIOL 4-88 (Calbiochem). Microscopy was performed using an inverted Zeiss Axioscope 2 Plus fluorescence microscope, and images were obtained with a Zeiss Axiocam digital camera using Axiovision software (version 3.1). The data were collected using a 63× oil immersion lens and DAPI filter (excitation 360/emission 460), and images were processed with Axiovision software and Adobe software (Photoshop 6.0).

### Scavenger Receptor Expression:

Cell surface CD36 and SR-A were measured by flow cytometry using CD36 (BD Biosciences Pharmingen, San Diego, Calif.) and CD204 (Serotec, Oxford, UK) specific monoclonal antibodies. See Liao et al. (2000) Arterioscler. Thromb. Vasc. Biol. 20: 1968-75; Hu et al. (1996) Blood 87: 2020-28. Murine macrophage J774A.1 cells were serum-starved overnight in media with 0.5% BSA. Cells (1×10⁶) treated with cholesterol ozonation products or cholesterol ozonation product complexed with LDL for specified times were then washed twice with PBS. After harvesting the cells by scraping, they were fixed with 6% paraformaldehyde for 20 min; Cells were then resuspended in PBS containing 2% heat-inactivated FCS, 0.05% NaN₃ and appropriate primary antibody, antibody conjugate or isotype control (1:100 dilution) and incubated on ice for 30 min. Expression of either CD36 or SR-A was determined with CD36 (BD Biosciences Pharmingen, San Diego, Calif.) and CD204 (Serotec, Oxford, U.K.) specific monoclonal antibodies, respectively. After washing off the primary antibody, secondary antibodies were added, cells were washed again, then analyzed on a FACS calibur 2 (Becton Dickinson, Sparks, Md.) flow cytometer and analyzed by CellQuant software. Results were expressed as mean fluorescence intensity±standard error of the mean (SEM) of at least duplicate determinations. Significance was determined using a student two-tail t test, with (*) indicating p<0.05, and (**) indicating p<0.01 versus the control.

Chemotaxis and invasion assays were performed using a modified Boyden chamber migration assay. Zwirner et al. (1998) Eur. J. Immunol. 28: 1570-77; Wilkinson (1988) Methods Enzymol. 162: 38-50. Assays were set up on MultiScreen-MIC plates (Millipore, Billerica, Mass., USA) with polyvinylpyrrolidone-free polycarbonate membranes (5 μm pore size). Cells (murine macrophage J774A.1) were washed and resuspended in serum-free DMEM containing 0.2% BSA at 1×10⁶ cells/ml. Fifty microliters of cells were added to the upper wells and 150 μl of chemoattractant in DMEM/0.2% BSA were added to the lower wells of the microchamber. Incubation time was for 4 h at 37° C. in an 8% CO₂ atmosphere. After swiping cells off the upper side of the membrane, cells on the lower side were visualized by either staining and phase contrast microscopy, or fluorescence. Cells that had adhered to the membrane were stained using a DiffQuik staining kit (Dade Behring, Newark, Del.). Each experiment was performed in triplicate and the numbers of migrating cells were counted in five fields at ×200. Cells harvested with PBS-EDTA for fluorescent labeling were washed two times with PBS, then resuspended in serum-free medium containing calcein AM (5 μM). Cells were incubated for 30 min (37° C., 5% CO₂) with gentle intermittent mixing. Calcein-labeled cells were washed twice, and calcein fluorescence (ex=390 nm and em=460 nm) was measured using a SPECTRAmax™ GEMINI dual-scanning microplate spectrofluorometer. Fluorescence measurements were compared to a standard curve, generated by dilution of cells in PBS/BSA in 100 μl, dispensed into 96-well plates. Fluorescence was quantified as described above.

### Endothelial Adhesion Molecule Expression:

Endothelial cell surface adhesion molecule expression was measured by ELISA, using anti-human CD54 (ICAM-1), CD106 (VCAM-1) and CD62E (E-Selectin) antibodies (Leinco Technologies, Inc., St. Louis, Mo.). The ELISA method was modified from previously described procedures. Ng et al. (2003 J. Am. Coll. Cardiol. 42: 1967-74; Khan (1995) J. Clin. Invest. 95: 1262-70; Huang et al. (2004) Carcinogenesis 25: 1925-34. HAAE-1 cells grown in 96-well plates were exposed to vehicle, LDL (1-3 nmol/mg protein), CuOx-LDL (TBARS, 80-100 nmol/mg protein), cholesterol ozonation products 4a or 5a (25 μM/LDL) for appropriate times and washed once with PBS.

Cells were then incubated with antibodies to VCAM-1, E-selectin, or ICAM-1 diluted 1:400 in PBS containing 5% FCS at 37° C. for 30 min. Wells were washed two times with PBS, then incubated with horseradish peroxidase (HRP)-conjugated goat anti-mouse IgG (Southern Biotechnology Associates, Birmingham, Ala., USA) in PBS/5% FCS at 37° C. for 30 min. The wells were washed four times with PBS, then incubated with H₂O₂/3,3′,5,5′-tetramethyl-benzidine peroxidase substrate (Pierce Chemical, Rockford, Ill., USA) for 30 min in the dark. The reaction was stopped by adding 25 μl of 8 N H₂SO₄, and the plates read on a microplate reader, blanking on wells stained with only secondary antibody. Each assay was performed in triplicate and the data are reported as the mean±SEM as a percentage of the expression level of the vehicle. To study the effect of cholesterol ozonation product 5a concentration (5-50 μM) on E-selectin expression, the assay was performed in an identical fashion as described above, with each point being the mean±SEM of at least duplicate measurements.

### Monocyte Morphology Changes

THP-1 monocytes in suspension were plated in 8-well chambered slides. Cells were incubated with vehicle (DMEM and 0.4% DMSO), cholesterol, 7-ketocholesterol (7-KC), or cholesterol ozonolysis products 4a or 5a (12 and 25 μM), and examined for morphological changes over a seven day incubation period at 37° C. Media containing appropriate treatment compounds was replaced after 4 days. After 4 days treatment, morphologic changes of the adherent cells were assessed by phase-contrast microscopy and photographed using an Olympus Microfire digital camera.

### Statistical Analyses:

In all cases, data were statistically analyzed by a two-tailed, paired Student's t-test in Microsoft EXCEL software. A value of P≦0.05 was taken as significant. Data given represent means±standard error.

### RESULTS & DISCUSSION

Previous Examples have shown that cultured macrophages treated with cholesterol ozonolysis products 4a and 5a and low density lipoprotein (LDL) exhibit a foamy cellular morphology associated with extensive lipid-loading. To further investigate this phenomenon and gain insight into the course of cholesterol ozonation product internalization, macrophages were treated with dansylated cholesterol ozonation product 4e, 5d, 5e or 9c and followed uptake by fluorescence microscopy. The dansyl cholesterol ozonation products 4e, 5d, 5e or 9c are as shown above.

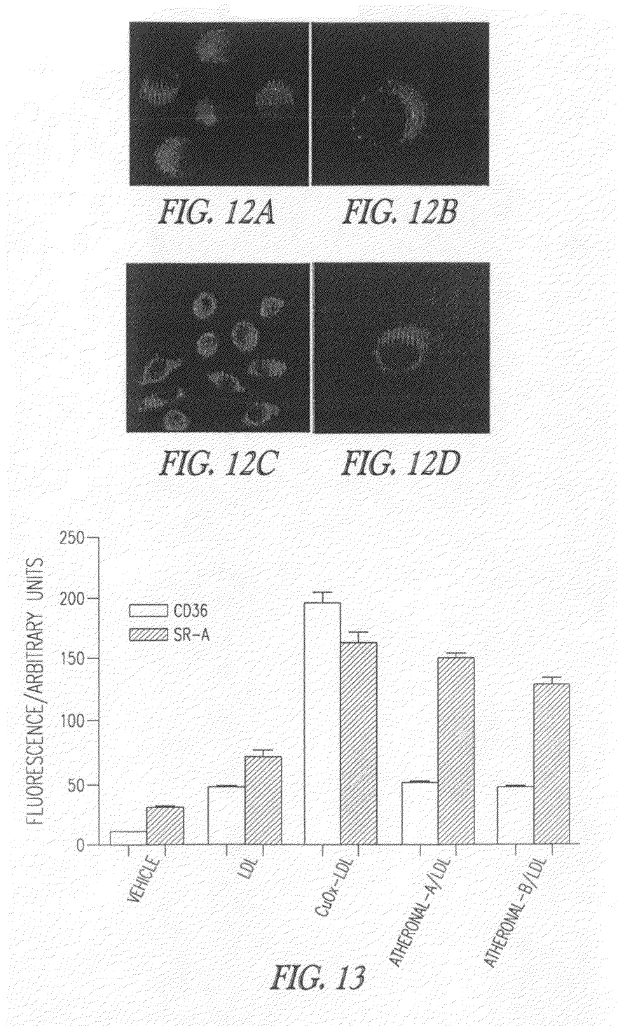

These experiments revealed that significant accumulation of cholesterol ozonation products, in macrophage cytosol, occurs after only 5 min (FIG. 12A). After one hour of exposure, significant perinuclear localization occurs (FIG. 12B).

Cholesterol ozonation product 4a and 5a were efficiently taken up by macrophages even when not complexed with LDL. This LDL-independent uptake may be of particular relevance in vivo because, under conditions where the cholesterol ozonation products may be generated in an extracellular compartment that is lacking functional LDL, such as is the case within the arterial intima of an atherosclerotic artery, these molecules may still accumulate in the cell, presumably by a receptor-independent pathway and, hence, have their intracellular effects on the macrophage function. In addition, the initial punctate cytosolic accumulation of the dansylated cholesterol ozonation product 5e, followed by its perinuclear accrual within the cell suggests that 5e is not being esterified and stored in lipid droplets, which would be localized throughout the cytosol.

Such uptake of cholesterol ozonation products can impact macrophage function downstream, regardless of whether the compounds are produced by means of extracellular oxidation of cholesterol or generated within macrophages and released upon necrosis. Moreover, the initial cytosolic accumulation of cholesterol ozonation products followed by their accrual within the cell suggests a mechanism of lipid-loading exists that is linked, by some as yet unknown mechanism, to perturbation of the normal endosomal recycling of cholesterol. Ridgway et al. (1992) J. Cell. Biol. 116: 307-19; Johansson et al. (2003) Mol. Biol. Cell. 14:903-15.

Class A scavenger receptor (SR-A) and CD36 are the main surface receptors responsible for macrophage internalization of modified LDL and oxysterols. Huh et al. (1996) Blood 87: 2020-28; Nicholson et al. (2001 Ann. N.Y. Acad. Sci. 947:224-28; Kunjathoor et al. (2002) J. Biol. Chem. 277:49982-88. Both receptors are upregulated in the presence of ox-LDL. However, SR-A is upregulated to a higher-degree than CD36 in the presence of acetylated-LDL. Yoshida et al. (1998) Artioscler. Thromb. Vasc. Biol. 18: 794-802; Han et al. (1997) J. Biol. Chem. 272: 21654-59; Nakagawa et al. (1998) Arterioscler. Thromb. Vasc. Biol. 18: 1350-57.

Treatment of J774A.1 macrophages with LDL and either cholesterol ozonolysis products 4a or 5a leads to significant upregulation of SR-A (approximately 3-fold, P<0.05), as measured by both indirect flow cytometry (FIG. 13) and immunoblotting of cellular lysates. However, no significant increase in LDL receptor (CD36) was observed when cholesterol ozonation products were complexed with LDL. Treatment of J774A.1 macrophages with CuOx-LDL (TBARS, 50-75 nmol/mg protein) resulted in a significant upregulation of both CD36 (about 4-fold increase over native LDL) and SR-A (about 3-fold increase over native LDL). In contrast, treatment of cultured macrophages with cholesterol ozonolysis products 4a or 5a in the absence of LDL did not increase expression of either receptor.

SR-A and CD36 are the main surface receptors responsible for macrophage internalization of modified LDL and oxysterols. It has been shown that both surface receptors are upregulated in the presence of oxidized low density lipoprotein (ox-LDL) (Yoshida et al. Arterioscler. Thromb. Vasc. Biol. 18: 794-802 (1998); Han et al., J. Biol. Chem. 272: 21654-21659 (1997)). However, SR-A is upregulated to a higher degree than CD36 in the presence of acetylated LDL (Nakagawa et al., Arterioscler., Thromb., Vasc. Biol. 18:1350-1357 (1998). Thus, the upregulation of SR-A, but not CD36, in macrophages in response to treatment with LDL complexed with either cholesterol ozonolysis products 4a or 5a, suggests a route of internalization for the presumed Schiff base LDL-ozonolysis product adduct highly analogous to that of acetylated-LDL.

These data indicate that ozonolysis product 5a rapidly accumulates in the cytosol within macrophages in the absence of LDL (FIG. 12A). Moreover, the data also indicate that ozonolysis products 4a or 5a in the presence of LDL lead to the upregulation of SR-A while in the absence of LDL they do not (FIG. 13). Together, these data suggest that the ozonolysis products 4a or 5a are able to enter macrophages by two distinct mechanisms dependant upon whether or not they are complexed with LDL. When not in complex with LDL, the ozonolysis products 4a or 5a may enter the macrophage by passive diffusion, but when complexed with LDL, the ozonolysis products 4a or 5a may enter by a surface receptor-mediated route.

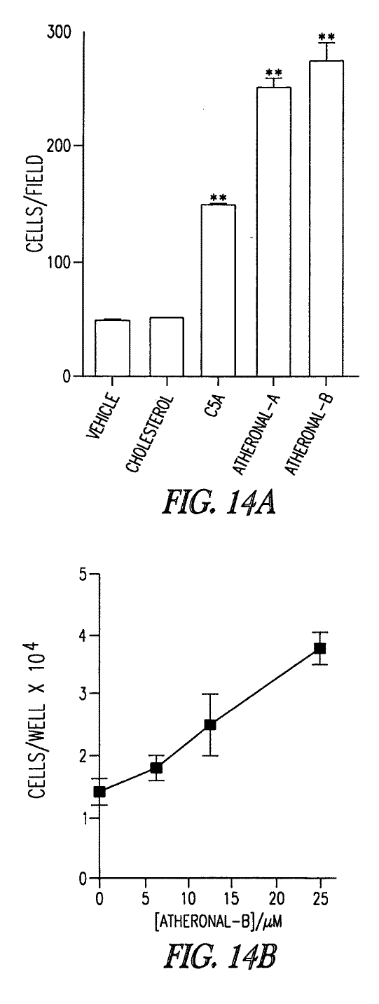

Both ozonolysis products 4a or 5a stimulate dose-dependent recruitment of macrophages as measured in a Boyden chamber migration assay (0-25 μM, P<0.001; FIGS. 14A and 14B). Interestingly, no significant migration of the macrophage cells occurs when ozonolysis products 4a or 5a are complexed with native LDL (100 μg/ml; TBARS, 1-4 nmol/mg protein). However, macrophage migration still occurs when ozonolysis products 4a or 5a are co-incubated with BSA migration. This suppression of cholesterol ozonolysis product-induced macrophage migration by LDL but not BSA further supports the conclusion that ozonolysis products 4a or 5a complexed to LDL behave in an analogous fashion to oxidatively modified LDL. This conclusion is supported by data showing that macrophages become unable to migrate after accumulation of oxidized LDL (Parthasarathy et al. (1988) Basic Life Sci 49: 375-80; Quinn et al. (1987) Proc. Natl. Acad. Sci. USA 84: 2995-98).

Macrophages are notoriously slow at replication; therefore, to increase local tissue levels at sites of inflammation, there are a number of naturally occurring chemotactic agents, such as monocyte chemoattractant protein 1 (MCP-1) or leukotriene B4 (LTB-4), that increase leukocyte accumulation at sites of inflammation. Macrophages are by far the major leukocyte present in atherosclerotic arteries, both during atherogenesis progression and at the ultimate thrombotic stage. Thus, the ability of ozonolysis products 4a or 5a to recruit macrophages may impact atherogenesis in multiple ways. First, macrophages that are already present within the artery wall may be recruited to sites were the cholesterol ozonolysis products are being generated extracellularly, leading to areas of high leukocyte density within the plaque, as is known to occur in unstable plaque margins. Alternatively, macrophages containing high levels of cholesterol ozonolysis products may necrose, leading to the release of cholesterol ozonolysis products and further recruitment of macrophages to the inflammatory foci. Finally, the cholesterol ozonolysis products may diffuse from vulnerable sites of the arterial walls, a possibility that is supported by the presence of cholesterol ozonolysis product 5a in the plasma of human atherosclerosis patients. These phenomena would result in a cycle of chemoattractant formation, macrophage chemotaxis, recruitment and activation, leading to further chemoattractant formation. This process may well promote the formation and progression of atherosclerotic lesions.

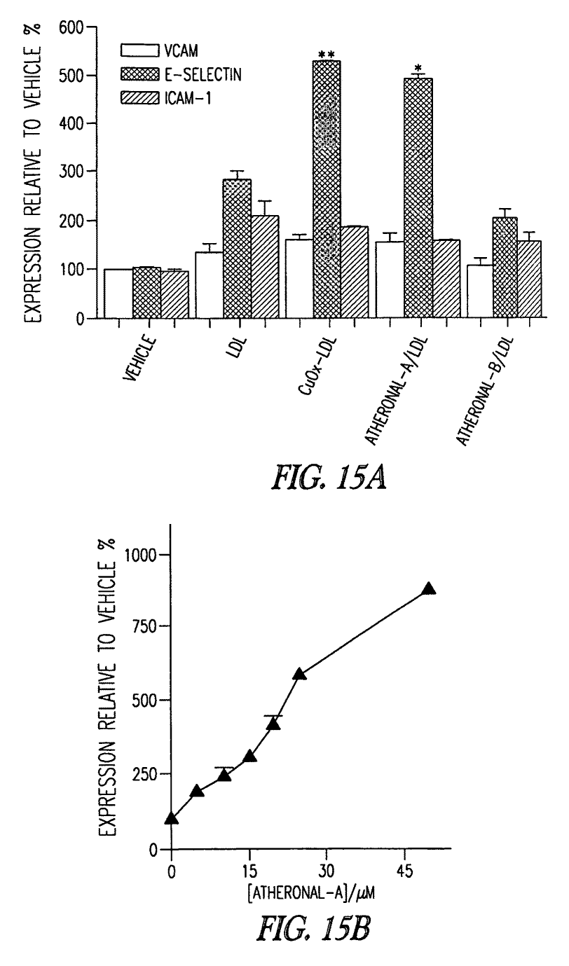

Treatment of vascular endothelial cell (HAAE-1) monolayers with ozonolysis product 4a (25 μM) in the presence of LDL stimulates a greater than 4-fold upregulation of expression of the adhesion molecule endothelial E-selectin relative to vehicle control (P<0.05, FIG. 15). In contrast, levels of the integrins, vascular cell adhesion molecule (VCAM)-1 and intercellular adhesion molecule (ICAM)-1, remain unchanged. The induction of E-selectin levels by ozonolysis product 4a was dose-dependent, with levels increasing from 2-fold at 1.25 μM ozonolysis product 4a to about 8-fold at 50 μM atheronal-A (FIG. 15B). This profile of E-selectin upregulation, coupled with the absence of an effect upon integrin expression, was the same as observed for CuOx-LDL [(TBARS, 80-100 nmol/mg of protein), p<0.005]. Incubation of endothelial cells with ozonolysis product 5a and LDL resulted in upregulation of E-selectin levels (1.8-fold), relative to the vehicle control, similar to what was observed with native LDL (about 3-fold increase, multiple experiments tested in triplicate, FIG. 15). Administration of ozonolysis product 4a or 5a (not complexed with LDL), cholesterol (data not shown), or the vehicle resulted in no effects on either selectin or integrin expression.

Given that ozonolysis product 4a, when in complex with LDL, causes a significant and dose-dependent increase in E-selectin upregulation, cell-cell adhesion was then assessed using fluorescence spectroscopy. These experiments revealed that neither ozonolysis product 4a nor 5a, alone or in complex with LDL, nor CuOx-LDL significantly increased adhesion of the cultured U-937 monocytes cells to endothelial cell monolayers, above that of LDL alone. When the effects of ozonolysis product 4a and 5a complexed to LDL on aortic endothelial cell adhesion are taken together, they are highly analogous to that of CuOx-LDL (Khan et al. (1995) J. Clin. Invest. 95: 1262-70; Vielma et al. (2004) J. Lipid Res. 45: 873-80). The profile of selectin upregulation with no effect on integrin expression and no significant increase in equilibrium binding of monocytes to endothelial cells is consistent with an induction of weak leukocyte-endothelial interactions and is the classic model for induction of leukocyte rolling rather than strict adhesion (Charo et al., J. Biol. Chem. 262: 9935-9938 (1987) a biological effect that is important in the early stages of inflammatory artery disease.

Previous work has demonstrated that the oxysterols 7-ketocholesterol, 7-hydroxycholesterol and 22(R)-hydroxycholesterol can induce monocyte differentiation, while others, such as 25-hydroxycholesterol, cannot (Hayden et al. (2002) J. Lipid Res. 43: 26-35). Therefore the monocyte differentiation profile of ozonolysis products 4a or 5a was examined.

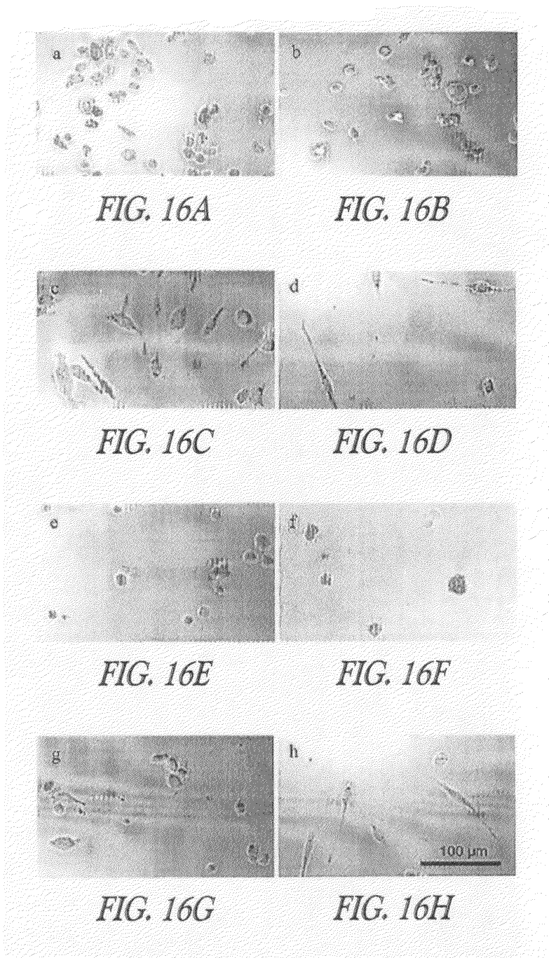

Initial studies studying the effects of cholesterol 5,6-secosterols on cultured human monocytes THP-1 cell differentiation demonstrated that these cells, originally in suspension, began to aggregate in clusters after 24-48 h of ozonolysis product 5a treatment. With a longer duration of exposure (about 4 days) with ozonolysis product 5a (12.5 and 25 μM), a population of cells began to adhere. Within 7 days of culture, the adherent THP-1 cells displayed hypertrophy, developed cytoplasmic vacuoles, and formed extended processes, all characteristic of mature macrophages (FIG. 16G-H). These effects of ozonolysis product 5a on THP-1 differentiation were exactly mimicked in both the extent and time frame to the positive control of 7-KC (25 μM, FIG. 16C-D). In contrast, THP-1 cells treated with either vehicle alone (data not shown), cholesterol (25 μM, FIG. 16A-B), or ozonolysis product 4a (25 μM, FIG. 16E-F) neither became adherent nor developed the aforementioned morphological changes. Thus, the differentiation activity of ozonolysis product 5a, especially when coupled with naturally occurring chemokines that recruit monocytes, e.g. MCP-1, serves to increase the numbers of functional macrophages within the inflammatory artery wall.

The ozonolysis product 5a-induced differentiation of the THP-1 cells occurred over a similar time course of human monocyte differentiation in vitro, typically requiring 4-7 days for THP-1 cell-derived macrophages to develop. In addition, the concentration of ozonolysis product 5a, that causes the determined THP-1 cell differentiation, 25 μM (10.4 μg/mL), is of the same order required for THP-1 cell differentiation by the oxysterol 7-KC, suggesting that they are equipotent in this regard (Hayden et al., J. Lipid Res. 43: 26-35 (2002)). The mechanism by which ozonolysis product 5a induces macrophage differentiation is unclear. However, on the basis of the fact that induction of cell adherence and morphological changes is relatively slow, a mechanism that involves cytokine induction and release seems plausible. It has been previously shown that other oxysterols, including 7-KC, are capable of producing proinflammatory cytokines in other vascular cells, such as monocyte colony-stimulating factor (M-CSF) and granulocyte-macrophage colony-stimulating factor (GM-CSF), if differentiation is slow. The fact that ozonolysis product 5a triggers monocyte to macrophage differentiation whereas ozonolysis product 4a does not reveals that, while the cholesterol ozonolysis products share the same chemical composition and a similar structure, they can behave as distinct molecular species.

Studies have revealed that the levels of oxysterols are concentrated in foam cells at levels several-fold higher than in plaque as a whole. It has been shown that oxysterols can be present in levels of as much as 1% of the total cholesterol within atherosclerotic plaque and within foam cells isolated from human atherosclerotic plaque (Brown et al. J. Lipid Res. 38: 1730-1745 (1997)). For cholesterol ozonation products, the absolute concentrations within diseased atherosclerotic arteries, with respect to cholesterol, are not known with certainty at present. However, as described in previous Examples, plasma levels of ozonolysis product 5a can vary from 20 to 500 nM (in cohorts of healthy patients and patients with advanced carotid disease). Given what is known about oxysterol levels in general, it is possible that within the atherosclerotic artery the levels of the ozonolysis products will be significantly higher. The experiments performed in this Example focus on ozonolysis product concentration ranges of 3-25 μM that are considered more reflective of levels within the atherosclerotic plaque and within foam cells found in the plaque rather than equilibrium plasma levels. These levels are considered valid because all of the studies performed are investigating effects that are either triggered within plaque material, such as uptake into macrophages either directly or via macrophage surface receptor up-regulation and monocyte to tissue macrophage differentiation, or can be triggered by the leakage of the ozonolysis products from the plaque material to intimately associated cells, such as the vascular endothelial cells.

This Example therefore demonstrates that the cholesterol ozonolysis products 4a or 5a, that are present in atherosclerotic plaque material, possess biological effects that may significantly impact atherogenesis and the progression of atherosclerosis. Ozonolysis products 4a or 5a facilitate macrophage recruitment to vascular tissue, via chemotaxis and endothelial adhesion molecule upregulation. In addition, these ozonolysis products trigger monocyte to macrophage differentiation, and macrophage foam cell formation via scavenger receptor uptake of ozonolysis product-modified LDL. In addition, the cholesterol ozonation products 4a and 5a enter macrophages by a receptor-independent process. All of these cholesterol ozonation product-induced effects are critical pathological processes ongoing in the context of inflammatory artery disease.

The inventors have shown in other Examples that activation of inflammatory cells contributes to the acute production of ozonolysis products 4a or 5a in excised atherosclerotic arteries. However it is also possible that ozonolysis products 4a or 5a arise chronically, in part, from lung exposure to ozone derived from environmental pollution. As such, ozonolysis products 4a or 5a may be a heretofore unrecognized chemical player in the known linkage between environmental pollution and cardiovascular disease.

### REFERENCES

1. P. Wentworth Jr. et al., Science 298, 2195 (2002).
2. B. M. Babior, C. Takeuchi, J. Ruedi, A. Guitierrez, P. Wentworth Jr., Proc. Natl. Acad. Sci. U.S.A. 100, 3920 (2003).
3. P. Wentworth Jr. et al., Proc. Natl. Acad. Sci. U.S.A. 100, 1490 (2003).
4. R. Ross, New Engl. J. Med 340, 115 (1999).
5. G. K. Hansson, P. Libby, U. Schönbeck, Z.-Q. Yan, Circ. Res. 91, 281 (2002).
6. D. Steinberg, J. Biol. Chem. 272, 20963 (1997).
7. D. Steinberg, S. Parthasarathy, T. E. Carew, J. C. Khoo, J. L. Witztum, New Engl. J. Med. 320, 915 (1989).
8. U. P. Steinbrecher, S. Parthasarathy, D. S. Leake, J. L. Witzum, D. Steinberg, Proc. Natl. Acad. Sci. U.S.A. 81, 3883 (1984).
9. K. Takeuchi, S. Kutsuna, T. Ibusuki, Anal. Chim. Acta 230, 183 (1990).
10. K. Takeuchi, 1. Takeuchi, Anal. Chem. 61, 619 (1989).
11. M. J. Steinbeck, A. U. Khan, M. J. Karnovsky, The Journal of Biological Chemistry 267, 13425 (1992).
12. H. Hietter, P. Bischoff, J. P. Beck, G. Ourisson, B. Luu, Cancer Biochem. Biophys. 9, 75 (1986).
13. J. L. Lorenso, M. Allorio, F. Bernini, A. Corsini, R. Fumagalli, FEBS Lett. 218, 77 (1987).
14. D. M. Small, Arteriosclerosis 8, 103 (1988).
15. J. Gumulka, J. St-Pyrek, L. L. Smith, Lipids 17, 197 (1982).
16. J. Gumulka, L. L. Smith, J. Am. Chem. Soc. 105, 1972 (1983).
17. K. Jaworski, L. L. Smith, J. Org. Chem 53, 545 (1988).
18. Z. Paryzek, J. Martynow, W. Swoboda, J. Chem. Soc. Perkin Trans. 1, 1222 (1990).
19. J. W. Cornforth, G. D. Hunter, G. Popjak, Biochem. J. 54, 590 (1953).
20. K. Wang, E. Bermudez, W. A. Pryor, Steroids 58, 225 (1993).
21. E. Lund, 1. Björkhem, Acc. Chem. Res. 28, 241 (1995).
22. T. Miyamoto, K. Kodama, Y. Aramaki, R. Higuchi, R. W. M. Van Soest, Tetrahedron Letter 42, 6349 (2001).
23. J. A. Levy, M. Virolainen, V. Defendi, Cancer 22, 517 (1968).
24. A. Weiss, R. L. Wiskocil, J. D. Stobo, J. Immunol. 133, 123 (1984).
25. J. Folkman, C. C. Haudenschild, B. R. Zetter, Proc. Natl. Acad. Sci. U.S.A. 76, 5217 (1979).
26. P. Ralph, M. A. Moore, K. Nilsson, J. Exp. Med. 143, 1528 (1976).
27. I. N. Mbawuike, H. B. Herscowitz, J. Leukoc. Biol. 46, 119 (1989).
28. J. T. N. Hiltermann et al., Free Radical Biology & Medicine 27, 1448 (1999).
29. M. Longphre, L.-Y. Zhang, J. R. Harkema, S. R. Kleeberger, J. Appl. Physiol. 86, 341 (1999).
30. M. T. Krishna et al., Eur. Respir. J. 11, 1294 (1998).
31. Q. Zhao, L. G. Simpson, K. E. Driscoll, G. D. Leikauf, American Journal of Physiology 274, L39 (1998).
32. M. D. Cohen, M. Sisco, Y. L1, J. T. Zelikoff, R. B. Schlesinger, Toxicology and Applied Pharmacology 171, 71 (2001).
33. J. L. Goldstein, Y. K. Ho, S. K. Basu, M. S. Brown, Proc. Natl. Acad. Sci. U.S.A. 76, 333 (1979).
34. W. L1, H. Dalen, J. W. Eaton, X.-M. Yuan, Arterioscler. Thromb. Vasc. Biol. 21, 1124 (2001).
35. W. Guo, J. D. Morrisett, M. E. DeBakey, G. M. Lawrie, J. A. Hamilton, Arterioscler. Thromb. Vasc. Biol. 20, 1630 (2000).
36. B. Liu, Z. Weishan, Tetrahedron Lett. 43, 4187 (2002).
37. K. Wang, E. Bermudez, W. A. Pryor, Steroids 58, 225 (1993).
38. T. Miyamoto, K. Kodama, Y. Aramaki, R. Higuchi, R. W. M. Van Soest, Tetrahedron Letter 42, 6349 (2001).
39. P. Yates, S. Stiveer, Can. J. Chem. 66, 1209 (1988).
40. A. Sevanian, A. R. Peterson, Proc. Natl. Acad. Sci. U.S.A. 81, 4198 (1984).
41. U. P. Steinbrecher, J. L. Wiztum, S. Parthasarathy, D. Steinberg, Arteriosclerosis 1, 135 (1987).
42. L. G. Fong, S. Parthasarathy, J. L. Wiztum, D. Steinberg, J. Lipid. Res. 28, 1466 (1987).
43. T. Parasassi et al., Free Radical Biol. & Med. 31, 82 (2001).
44. F. Ursini, K. J. A. Davies, M. Maiorino, T. Parasassi, A. Sevanian, Trends in Mol. Med. 8, 370 (2002).
45. R. Brunelli et al., Biochemistry 39, 13897 (2000).
46. S. Lund-Katz, P. M. Laplaud, M. C. Phillips, M. J. Chapman, Biochemistry 37, 12867 (1998).
47. G. C. Chen et al., J. Biol. Chem. 269, 29121 (1994).
48. E. Lund, I. Björkhem, Acc. Chem. Res. 28, 241 (1995).
49. R. Ross, J. A. Glomset, New Engl. J. Med. 295, 369 (1976).
50. P. Wentworth Jr. et al., Science 293, 1806 (2001).
51. J.-L. Reymond, Y. Chen, J. Org. Chem. 60, 6970 (1995).
52. J. Gumulka, J. St-Pyrek, L. L. Smith, Lipids 17, 197 (1982).
53. P. Wentworth Jr. et al., Science 293, 1806 (2001).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method for identifying an agent that can inhibit recruitment of macrophages to atherosclerotic tissues that comprises contacting a macrophage with a test agent and observing whether the macrophage migrates toward a source of cholesterol ozonolysis products 4a or 5a:

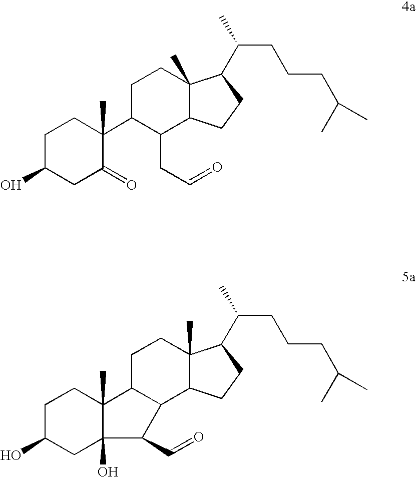

2. The method of claim 1, which further comprises quantifying a percentage of macrophages that migrate toward a source of cholesterol ozonolysis products 4a or 5a.

3. The method of claim 1, which further comprises comparing the percentage of macrophages that migrate toward a source of cholesterol ozonolysis products 4a or 5a with a control percentage of macrophages that migrate toward a source of cholesterol ozonolysis products 4a or 5a.

4. The method of claim 3, wherein the control percentage of macrophages that migrate toward a source of cholesterol ozonolysis products 4a or 5a is determined by observing a percentage of macrophages that migrate toward a source of cholesterol ozonolysis products 4a or 5a without exposing the macrophage to the test agent.

5. A method for identifying an agent that can inhibit atherosclerosis that comprises contacting an endothelial cell with a test agent and observing whether expression of E-selectin increases in the endothelial cell exposing the endothelial to cholesterol ozonolysis products 4a or 5a in the presence of LDL:

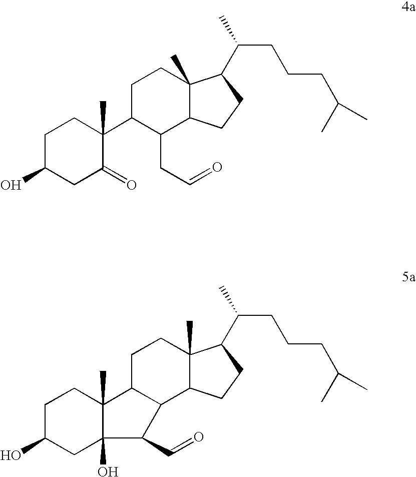

6. The method of claim 5, which further comprises quantifying E-selectin expression levels.

7. The method of claim 6, which further comprises comparing the quantified E-selectin expression levels with control quantified E-selectin expression levels.

8. The method of claim 7, wherein the control quantified E-selectin expression levels are determined by observing expression of E-selectin increases in the macrophage after exposing the macrophage to cholesterol ozonolysis products 4a or 5a in the presence of LDL without exposing the macrophage to the test agent.

9. The method of claim 7, wherein the control quantified E-selectin expression levels are determined by observing expression of E-selectin increases in the macrophage after exposing the macrophage to the test agent without exposing the macrophage to the 4a or 5a cholesterol ozonolysis products in the presence of LDL.

10. A method for identifying an agent that can inhibit monocyte differentiation into macrophages that comprises contacting a monocyte cell with a test agent and observing whether the monocyte differentiates into a macrophage, wherein the monocyte is cultured with cholesterol ozonolysis product 4a or 5a:

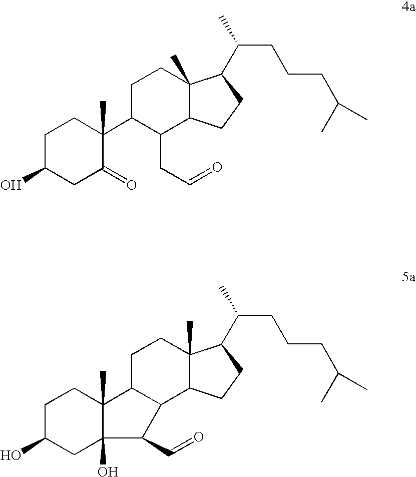

11. The method of claim 10, which further comprises determining a percentage of the monocytes that differentiate into macrophages.

12. The method of claim 11, which further comprises comparing the percentage of the monocytes that differentiate into macrophages with a control percentage of monocytes that differentiate into macrophages.

13. The method of claim 12, wherein the control percentage of monocytes that differentiate into macrophages is determined by observing a percentage of the monocytes that differentiate into macrophages after exposing the monocyte to cholesterol ozonolysis products 4a or 5a without exposing the monocyte to the test agent.

14. The method of claim 12, wherein the control percentage of monocytes that differentiate into macrophages is determined by observing a percentage of the monocytes that differentiate into macrophages after exposing the monocyte to the test agent without exposing the monocyte to the 4a or 5a cholesterol ozonolysis products.

15. An isolated dansyl derivative of cholesterol ozonolysis product consisting essentially of compound 5d or 5e.

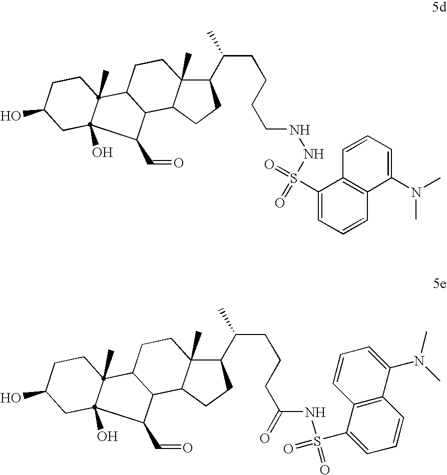

16. An isolated dansyl derivative of cholesterol consisting essentially of compound 9c or 9d. [chemical structure]
